(19) United States
(12) Patent Application Publication
(10) Pub. No.: US 2005/0154039 A1
(43) Pub. Date: Jul. 14, 2005

(54) 5-Sulphanyl-4h-1,2,4-triazole derivatives and their use as medicine

(76) Inventors: Marie-Odile Glacera Contour, Bondoufle (FR); Alban Sidhu, Vauhallan (FR); Pierre Roubert, Paris (FR); Christophe Thurieau, Paris (FR)

Correspondence Address: MUSERLIAN, LUCAS AND MERCANTI, LLP, 475 PARK AVENUE SOUTH, 15TH FLOOR, NEW YORK, NY, 10016, US

(21) Appl. No.: 10/496,820

(22) PCT Filed: Nov. 27, 2002
(86) PCT No.: PCT/FR02/04055

(30) Foreign Application Priority Data
Nov. 28, 2001 (FR) 01/15342
Jun. 21, 2002 (FR) 02/07697

(51) Int. Cl. A61K 31/4196; C07D 45/02; C07D 43/02; C07D 249/12

(52) U.S. Cl. 514/394; 548/264.4

(57) ABSTRACT

The invention concerns novel 5-sulphanyl-4<I>H</I>-1,2,4-triazole derivatives of formula (1), wherein: R₁, R₂ and R₃ represent variable groups and the methods for preparing them by liquid-phase parallel synthesis processes. Said product exhibit good affinity for certain sub-types of somatostatin receptors; they are particularly useful for treating pathological conditions or diseases wherein one (or more) somatostatin receptors is (are) involved. The invention also concerns pharmaceutical compositions containing said products and their use for preparing a medicine.

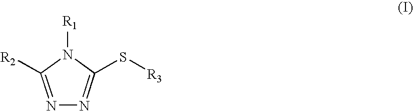

A subject of the present Application is new derivatives of 5-sulphanyl-4H-1,2,4-triazoles and their preparation processes by methods of liquid-phase parallel synthesis. These products having a good affinity for certain sub-types of somatostatin receptors, they are particularly useful for treating pathological conditions or diseases in which one (or more) somatostatin receptors is (are) involved. The invention also relates to pharmaceutical compositions containing said products and their use for the preparation of a medicament.

Somatostatin (SST) is a cyclic tetradecapeptide which was isolated for the first time from the hypothalamus as a substance which inhibits the growth hormone (Brazeau P. et al., Science 1973, 179, 77-79). It also operates as a neurotransmitter in the brain (Reisine T. et al., Neuroscience 1995, 67, 777-790; Reisine T. et al., Endocrinology 1995, 16, 427-442) The heterogeneity of the biological functions of somatostatin and the structure-activity relationship of its peptide analogues, have led to the discovery of 5 sub-types of receptors linked to the membrane (Yamada et al., Proc. Natl. Acad. Sci. U.S.A, 89, 251-255, 1992; Raynor, K. et al, Mol. Pharmacol., 44, 385-392, 1993). Molecular cloning has allowed it to be shown that the bioactivity of somatostatin depends directly on these five sub-types of receptors.

The functional roles of these receptors are currently being actively studied. Preferential activation of sub-types 2 and 5 has been associated with the suppression, in the adenomas secreting these hormones, of the growth hormone GH (acromegalia), of TSH and prolactin; but the precise role of each sub-type remains to be determined.

Among the pathological disorders associated with somatostatin (Moreau J. P. et al., Life Sciences 1987, 40, 419; Harris A. G. et al., The European Journal of Medicine, 1993, 2, 97-105), there can be mentioned the endocrine diseases linked to an excess of hormone such as growth hormone, insulin or glucagon. The compounds of the present invention are thus suitable for treating diseases such as acromegalia, hypophyseal adenomas, Cushing's disease, gonadotrophinomas and prolactinomas, diabetes and its complications, diabetic retinopathy, diabetic nephropathy, hyperthyroidism, gigantism. Gastrointestinal diseases, diseases associated with an exocrine or endocrine, gastric or pancreatic hypersecretion, or also with a release of various peptides of the gastrointestinal tract are also concerned. The compounds of the present invention are thus suitable for treating diseases such as endocrinic gastroenteropancreatic tumours including carcinoid syndrome, VIPoma, insulinoma, nesidioblastoma, hyperinsulinemia, glucagonoma, gastrinoma and Zollinger-Ellison's syndrome, GRFoma as well as acute bleeding of the oesophageal varices, gastroesophageal reflux, gastroduodenal reflux, pancreatitis, enterocutaneous and pancreatic fistulae but also diarrhoeas, disorders linked with gastrin-releasing peptide, secondary pathologies with intestinal grafts, portal hypertension as well as haemorrhages of the varices in patients with cirrhosis, gastro-intestinal haemorrhage, haemorrhage of the gastroduodenal ulcer, Crohn's disease, systemic scleroses, dumping syndrome, small intestine syndrome, hypotension, scleroderma and medullar thyroid carcinoma. The compounds of the present invention are suitable for treating diseases linked with cell hyperproliferation such as cancers and more particularly breast cancer, prostrate cancer, thyroid cancer, as well as pancreatic cancer and colorectal cancer, brain cancer, lung cancer, fibroses and more particularly fibrosis of the kidney, fibrosis of the liver, fibrosis of the lung, fibrosis of the skin, also fibrosis of the central nervous system as well as that of the nose and fibrosis induced by chemotherapy. Other therapeutic fields such as cephaleas including cephalea associated with hypophyseal tumors, pain, psychological alterations such as anxiety, depression and schizophrenia, chemotherapy, cicatrization of wounds, renal insufficiency resulting from delayed development, obesity and delayed development linked with obesity, delayed uterine development, dysplasia of the skeleton, Noonan's syndrome, sleep apnea syndrome, Graves' disease, polycystic disease of the ovaries, pancreatic pseudocysts and ascites, leukemia, meningioma, cancerous cachexia, inhibition of H pylori, psoriasis, as well as neurodegenerative diseases such as dementia, epilepsy or Alzheimer's disease. Osteoporisis can also be mentioned.

The Applicants have found that the compounds of the general formula described hereafter have an affinity and a selectivity for the somatostatin receptors. As somatostatin and its peptide analogues often have a poor bioavailability by oral route and a low selectivity (Robinson, C., Drugs of the Future, 1994, 19, 992; Reubi, J. C. et al., TIPS, 1995, 16, 110), said compounds, non-peptide agonists or antagonists of somatostatin, can be advantageously used to treat pathological states or diseases as presented above and in which one (or more) somatostatin receptors are involved. Preferably, said compounds can be used for the treatment of acromegalia, hypophyseal adenomas, gastric or pancreatic hypersecretions, gastroenteropancreatic tumours, cancers of the breast, of the prostate, of the thyroid, of the lung and of the fibroses.

The compounds of the invention are also analogues of urotensin II and are thus particularly useful for treating pathological conditions or diseases in which urotensin II is involved.

Different forms of urotensin II (U-II), a cyclic peptide sequenced more than 20 years ago, have been isolated in several species of fish and amphibians. These peptides show a capacity for contraction of the smooth muscles as well as a significant vasoconstrictor capacity. More recently, urotensin II was cloned in different species of mammals, including humans. Human urotensin (hU-II) is a cyclic undecapeptide which maintains the cyclic hexapeptidic part also present in the other animal forms of the protein (P. Grieco et al. Bioorg. Med. Chem. 2002, 10, 3731-3739). In humans U-11 has shown a significant vasoconstrictor effect on the veins and arteries in vitro. Furthermore, U-II and its receptor are present in the brain of rats, suggesting a possible neurotransmitter or neuromodulator role in the central nervous system (J. J. Maguire, A. P. Davenport Br J. Pharmacol 2002, 579-588).

The compounds of general formula described below, as analogues of urotensin II, can be used for treating pathological conditions linked to hypertension (portal, pulmonary, renal, cerebral), to cardiovascular disorders (cardiac hypertrophy, cardiac arrhythmia, angina), to pulmonary disorders (asthma), as well as to atherosclerosis and to strokes. Furthermore, U-II and its receptor being present in the central nervous system of mammals, the compounds of the invention can also be used in the treatment of anxiety, stress, schizophrenia, depression and alterations in the neuromuscular functions.

Therefore a subject of the present invention is the compounds of general formula 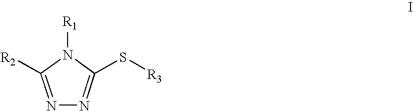
in racemic, enantiomeric form or all combinations of these forms, in which:
one of the R₁, R₂ or R₃ radicals represents a radical of formula —(CH₂)ₙ—[Q]ₚ(CH₂)ₘ—NXY or —(CH₂)ₙ—W
   W represents a heterocycloalkyl containing at least one nitrogen atom;
   Q represents —O—, —S—, —C(O)—NH—, —C(Z_{q})(Z_{q}′)—, aryl or (C₃-C₇)cycloalkyl; Z_{q} and Z_{q}′ represent, independently, the hydrogen atom, aryl optionally substituted by aryl, (C₃-C₇)cycloalkyl-alkyl, aralkyl, —C(O)O—R or —C(O)—NH—R′;
   R represents a (C₁-C₆)alkyl, aryl or aralkyl radical, aryl and aralkyl being optionally substituted by one or more identical or different substituents chosen from: (C₁-C₆)alkoxy, hydroxy, halo, nitro cyano, amino, (C₁-C₆)alkylamino and di((C₁-C₆)alkyl)amino;
   R′ represents a (C₁-C₆)alkyl, aryl, aralkyl, heteroaryl or heteroaryl-alkyl radical, the aryl, aralkyl, heteroaryl and heteroaryl-alkyl radicals being optionally substituted by one or more identical or different substituents chosen from: (C₁-C₆)alkoxy, hydroxy, halo, nitro cyano, amino, (C₁-C₆)alkylamino, di((C₁-C₆)alkyl)amino;
   X and Y represent, independently, the hydrogen atom, (C₁-C₆)alkyl, (C₁-C₆)alkoxy-carbonyl or a heteroaryl-alkyl, or X and Y form together with the nitrogen atom on which they are attached, a heterocycloalkyl optionally substituted by a (C₁-C₆)alkyl;
   p represents 0 or 1; n and m independently represent an integer from 0 to 6;
and the two other radicals represent, independently, a radical of formula —(CH₂)_{n′}[Q′]_{p′}[C(X′)(Y′)]_{m′}Z′
   Q′represents —O—, —S—, —C(O)—, —NH—, —CH═CH— or —C—C═;
   X′, Y′ and Z′ represent, independently, a hydrogen atom, (C₁-C₆)alkyl, (C₁-C₆) alkoxy, (C₁-C₆)alkoxy-carbonyl, cyano, amino, (C₁-C₆)alkylamino, di((C₁-C₆)alkyl)amino, (C₃-C₇)cycloalkyl, heterocycloalkyl, aryl or heteroaryl, or a radical of formula 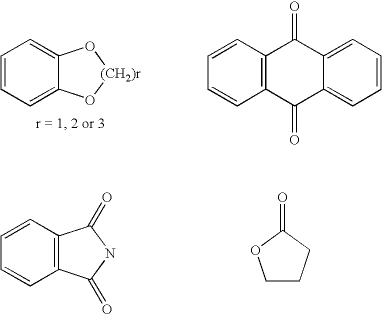
   the (C₃-C₇)cycloalkyl, heterocycloalkyl, aryl and heteroaryl radicals being optionally substituted by one or more identical or different substituents chosen from: (CH₂)_{q′}X″—Y″, hydroxy, halo, nitro, cyano, amino, (C₁-C₆)alkylamino and di((C₁-C₆)alkyl)amino;
   X″ represents —O—, —S—, —C(O)—, —C(O)—O—, —SO₂— or a covalent bond;
Y″ represents a (C₁-C₆)alkyl radical optionally substituted by one or more identical or different halo radicals; or aryl or heteroaryl radical optionally substituted by one or more identical or different substituents chosen from: (C₁-C₆)alkoxy, hydroxy, halo, nitro cyano, amino, (C₁-C₆)alkylamino and di((C₁-C₆)alkyl)amino;
   p′ represents 0 or 1, and n′, m′ and q′ represent, independently, an integer from 0 to 6;
excluding the compounds in which
i) R₁ represents (CH₂)₂—W and W representing morpholino or piperazinyl, R₂ phenyl, m-chlorophenyl or 4-pyridyl, and R₃ the hydrogen atom;
   ii) R₁ represents (CH₂)₂—W and W representing pyrrolidinyl, R₂ p-chlorophenyl and R₃ the hydrogen atom;
or their addition salts with pharmaceutically acceptable mineral or organic acids.

In the definitions indicated above, the expression halo represents the fluoro, chloro, bromo or iodo radical, preferably chloro, fluoro or bromo. The expression alkyl (when it is not specified otherwise), preferably represents a linear or branched alkyl radical having 1 to 6 carbon atoms, such as the methyl, ethyl, propyl, isopropyl, butyl, isobutyl, sec-butyl and tert-butyl, pentyl or amyl, isopentyl, neopentyl, hexyl or isohexyl radicals. Moreover, in the present Application, the —(CH₂)_{n′}— radical represents a hydrocarbon-containing chain of n′ carbon atoms which can be linear or branched; this —(CH₂)_{n′}— radical can thus represent the alkyl radicals as defined above.

The term (C₃-C₇)cycloalkyl designates a monocyclic carbon-containing system containing 3 to 7 carbon atoms, and preferably the cyclopropyl, cyclobutyl, cyclopentyl or cyclohexyl rings. The expression heterocycloalkyl designates a saturated cycloalkyl containing 2 to 7 carbon atoms and at least one heteroatom. This radical can contain several identical or different heteroatoms. Preferably, the heteroatoms are chosen from oxygen, sulphur or nitrogen. As examples of heterocycloalkyl, there can be mentioned rings containing at least one nitrogen atom such as pyrrolidine, pyrrolidinone, imidazolidine, pyrrazolidine, isothiazolidine, thiazolidine, isoxazolidine, piperidine, piperazine or morpholine, or tetrahydrofliran or tetrahydrothiophene.

The alkoxy radicals can correspond to the alkyl radicals indicated above such as for example the methoxy, ethoxy, propyloxy or isopropyloxy radicals but also linear, secondary or tertiary butoxy, pentyloxy. The term alkoxycarbonyl preferably designates the radicals in which the alkoxy radical is as defined above such as for example methoxycarbonyl, ethoxycarbonyl.

The expression aryl represents an aromatic radical, constituted by a ring or condensed rings, such as for example the phenyl, naphthyl or fluorenyl radical. The expression heteroaryl designates an aromatic radical, constituted by a ring or condensed rings, with at least one ring containing one or more identical or different heteroatoms chosen from sulphur, nitrogen or oxygen. As an example of a heteroaryl radical, the thienyl, furyl, pyrrolyl, imidazolyl, pyrazolyl, isothiazolyl, thiazolyl, isoxazolyl, oxazolyl, triazolyl, thiadiazolyl, pyridyl, pyrazyl, pyrimidyl, quinolyl, isoquinolyl, quinoxalyl, naphthyridyl, xanthenyl, benzothienyl, benzofuiryl, indolyl and benzoxadiazolyl radicals can be mentioned. The terms aralkyl (arylalkyl), cycloalkyl-alkyl and heteroaryl-alkyl preferably designate the radicals in which the aryl, cycloalkyl and heteroaryl radical respectively, and alkyl are as defined above; as an example of arylalkyl, benzyl and phenethyl can be mentioned.

The terms alkylamino and dialkylamino preferably designate the radicals in which the alkyl radicals are as defined above, such as for example methylamino, ethylamino, dimethylamino, diethylamino or (methyl)(ethyl)amino.

Preferably, the invention relates to compounds of formula I as defined above and in which
R₁ represents a radical of formula —(CH₂)ₙ—[Q]ₚ—(CH₂)ₘ—NXY
   Q represents aryl or (C₃-C₇)cycloalkyl;
   X and Y represent, independently, the hydrogen atom, a (C₁-C₆)alkyl, or X and Y form together with the nitrogen atom on which they are attached, a heterocycloalkyl optionally substituted by a (C₁-C₆)alkyl;
   p represents 0 or 1, and n and m represent, independently, an integer from 0 to 6;
R₂ represents a radical of formula (CH₂)_{n′}[Q′]_{p′}[C(X′)(Y′)]_{m′}Z′
   Q′ represents —O—;
   X′ represents the hydrogen atom,
   Y′ and Z′ represent, independently, a hydrogen atom, (C₁-C₆)alkyl, cyano, amino, (C₃-C₇)cycloalkyl, aryl or heteroaryl;
   the aryl and heteroaryl radicals being optionally substituted by one or more identical or different substituents chosen from: —(CH₂)_{q′}—X″—Y″, hydroxy, halo, nitro, amino, (C₁-C₆)alkylamino, di((C₁-C₆)alkyl)amino;
   X″ represents —O—, —S— or a covalent bond;
   Y″ represents a (C₁-C₆)alkyl radical optionally substituted by one or more identical or different halo radicals, or aryl radical optionally substituted by one or more identical or different halo radicals;
   p′ represents 0 or 1; n′ represents 0, 1 or 2; and m′ and q′ represent an integer from 0 to 6;
R₃ represents a radical of formula —(CH₂)_{n′}[Q′]_{p′}[C(X′)(Y′)]_{m′}Z′
   Q′ represents —O—, —C(O)—, —CH═CH— or —C≡C—;
   X′ represents the hydrogen atom;
   Y′ and Z′ represent, independently, a hydrogen atom, (C₁-C₆)alkyl, (C₁-C₆)alkoxy-carbonyl, (C₃-C₇)cycloalkyl, aryl or heteroaryl, or a radical of formula 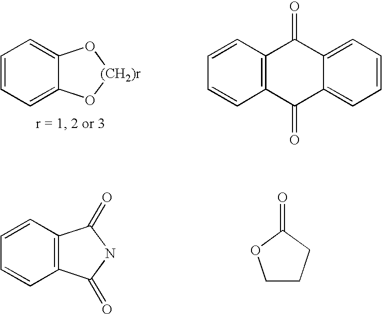
   the aryl and heteroaryl radicals being optionally substituted by one or more identical or different substituents chosen from: —(CH₂)_{q′}—X″—Y″, halo, nitro, cyano, di((C₁-C₆)alkyl)amino;
   X″ represents —O—, —C(O)—, —C(O)—O—, —SO₂— or a covalent bond;
   Y″ represents a (C₁-C₆)alkyl radical optionally substituted by one or more identical or different halo radicals, or an aryl radical;
   p′ represents 0 or 1; n′, m′ and q′ represent an integer from 0 to 6.
and more particularly
   the aryl radical represented by Q is the phenyl radical; the (C₃-C₇)cycloalkyl radical represented by Q is the cyclohexyl radical;
   the heterocycloalkyl that X and Y form, together with the nitrogen atom on which they are attached, is chosen from: pyrrolidine, piperidine, piperazine and morpholine;
   the (C₃-C₇)cycloalkyl represented independently by Y′ and Z′, is the cyclohexyl radical;
   the aryl radical represented independently by Y′ and Z′, is chosen from: phenyl, naphthyl and fluorenyl;
   the heteroaryl radical represented independently by Y′ and Z′ of the R₂ radical is chosen from: thienyl, furyl, benzothienyl, pyridyl, indolyl, thiadiazolyl, quinolyl, isoquinolyl, quinoxalyl, xanthenyl and naphthyridyl;
   the heteroaryl radical represented independently by Y′ and Z′ of the R₃ radical is chosen from: benzothienyl, furyl, indolyl and isoxazolyl; and
   the aryl radical represented by Y″ is the phenyl radical.

Preferably, the invention also relates to compounds of formula I as defined above and in which
R₁ represents a radical of formula —(CH₂)_{n′}[Q′]_{p′}[C(X′)(Y′)]_{m′}Z′
   X′ represents the hydrogen atom;
   Y′ and Z′ represent, independently, a hydrogen atom, (C₁-C₆)alkyl or aryl,
   the aryl radical being optionally substituted by one or more identical or different substituents chosen from: —(CH₂)_{q}—X″—Y″, halo, amino;
   X″ represents a covalent bond;
   Y″ represents an aryl radical;
   p′ represents 0, n′ represents 0 or 1, and m′ represents an integer from 0 to 6;
R₂ represents a radical of formula —(CH₂)ₙ—[Q]ₚ—(CH₂)ₘ—NXY or —(CH₂)ₙ—W
   W represents a heterocycloalkyl containing at least one nitrogen atom;
   Q represents —C(Z_{q})(Z_{q′})—;
   Z_{q} represents the hydrogen atom;
   Z_{q′} represents the hydrogen atom, aryl optionally substituted by aryl, (C₃-C₇)cycloalkyl-alkyl or aralkyl;
   X and Y represent, independently, the hydrogen atom, (C₁-C₆)alkyl or (C₁-C₆)alkoxy-carbonyl;
   p represents 0 or 1, and n represents 0 or 1, and m represents an integer from 0 to 6;
R₃ represents a radical of formula —(CH₂)_{n′}[Q′]_{p′}[C(X′)(Y′)]_{m′}Z′ in which
   Q′ represents —O—, —C(O)—, —CH═CH— or —C≡C—
   X′ represents the hydrogen atom;
   Y′ and Z′ represent, independently, a hydrogen atom, (C₁-C₆)alkyl, (C₁-C₆)alkoxy-carbonyl, (C₃-C₇)cycloalkyl, aryl or heteroaryl, or a radical of formula 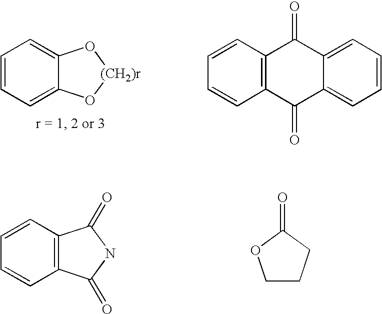
   the aryl and heteroaryl radicals being optionally substituted by one or more identical or different substituents chosen from: —(CH₂)_{q}—X″—Y″, halo, nitro, cyano, di((C₁-C₆)alkyl)amino;
   X″ represents —O—, —C(O)—, —C(O)—O—, —SO₂— or a covalent bond;
   Y″ represents a (C₁-C₆)alkyl radical optionally substituted by one or more identical or different halo radicals, or an aryl radical;
   p′ represents 0 or 1; n′ and m′ represent an integer from 0 to 6;
and more particularly
   the aryl radical represented independently by Y′ and Z′ of the R₁ radical is chosen from phenyl and naphthyl;
   the heterocycloalkyl represented by W, is the piperidine or pyrrolidine ring;
   the aryl radical represented by Z_{q}′, is the phenyl or naphthyl radical;
   the aryl substituent of the aryl radical represented by Z_{q}′, is the phenyl radical;
   the arylalkyl radical represented by Z_{q}′, is the benzyl radical;
   the (C₃-C₇)cycloalkyl of the —(C₃-C₇)cycloalkyl-alkyl radical represented by Z_{q}′, is the cyclohexyl;
   the (C₃-C₇)cycloalkyl represented independently by Y′ and Z′, is the cyclohexyl radical;
   the aryl radical represented independently by Y′ and Z′ of the R₃ radical is chosen from: phenyl, naphthyl and fluorenyl;
   the heteroaryl radical represented independently by Y′ and Z′ of the R₃ radical is chosen from: benzothienyl, furyl, indolyl and isoxazolyl; and
   the aryl radical represented by Y″ is the phenyl radical.

Preferably, the invention also relates to compounds of formula I as defined above and in which
R₁ represents a radical of formula (CH₂)_{n′}[Q′]_{p′}[C(X′)(Y′)]_{m′}Z′
   X′ represents the hydrogen atom;
   Y′ and Z′ represent, independently, a hydrogen atom, (C₁-C₆)alkyl, or aryl optionally substituted by one or more identical or different substituents chosen from: —(CH₂)_{q}—X″—Y″, halo, amino;
   X″ represents a covalent bond;
   Y″ represents an aryl radical;
   p′ represents 0, n′ represents 0 or 1, and m′ represents an integer from 0 to 6;
R₂ represents a radical of formula —(CH₂)_{n′}[Q′]_{p′}[C(X′)(Y′)]_{m′}Z′
   Q′ represents —O—;
   X′ represents the hydrogen atom;
   Y′ and Z′ represent, independently, a hydrogen atom, (C₁-C₆)alkyl, cyano, amino, (C₃-C₇)cycloalkyl, aryl or heteroaryl;
   the aryl and heteroaryl radicals being optionally substituted by one or more identical or different substituents chosen from: —(CH₂)_{q}—X″—Y″, hydroxy, halo, nitro, amino, (C₁-C₆)alkylamino, di((C₁-C₆)alkyl)amino;
   X″ represents —O—, —S— or a covalent bond;
   Y″ represents a (C₁-C₆)alkyl radical optionally substituted by one or more identical or different halo radicals, or an aryl radical optionally substituted by one or more identical or different halo radicals;
   p′ represents 0 or 1; n′ represents 0, 1 or 2; and m′ represents an integer from 0 to 6;
R₃ represents a radical of formula —(CH₂)ₙ—[Q]ₚ—(CH₂)ₘ—NXY or —(CH₂)ₙ—W
   W represents a heterocycloalkyl containing at least one nitrogen atom;
   Q represents —C(O)—NH—;
   X and Y represent, independently, the hydrogen atom, (C₁-C₆)alkyl or a heteroaryl-alkyl, or X and Y form together with the nitrogen atom on which they are attached, a heterocycloalkyl optionally substituted by (C₁-C₆)alkyl;
   p represents 0 or 1, and n represents 0 or 1 and m represents an integer from 0 to 6;
and more particularly
   the aryl radical represented independently by Y′ and Z′ of the R₁ radical is chosen from phenyl and naphthyl;
   the heterocycloalkyl represented by W, is the piperidine ring;
   the (C₃-C₇)cycloalkyl represented independently by Y′ and Z′, is the cyclohexyl radical;
   the aryl radical represented independently by Y′ and Z′ of the R₂ radical is chosen from: phenyl, naphthyl and fluorenyl;
   the heteroaryl radical of the heteroaryl-alkyl radical represented independently by X and Y, is the pyridine ring;
   the heterocycloalkyl formed by X and Y together with the nitrogen atom on which they are attached, is chosen from: piperazine and pyrrolidine;
   the heteroaryl represented independently by Y′ and Z′ of the radical R₂ is chosen from: thienyl, furyl, benzothienyl, pyridinyl, indolyl and thiadiazolyl; and
   the aryl radical represented by Y″ is the phenyl radical.

A more particular subject of the present invention is the compounds of general formula I as defined above in which
R₁ represents a radical of formula —(CH₂)ₙ—[Q]ₚ—(CH₂)ₘ—NXY in which
   Q represents the cyclohexyl radical;
   X and Y represent, independently, the hydrogen atom, a (C₁-C₆)alkyl, or X and Y form together with the nitrogen atom on which they are attached, the piperidine ring;
   n represents 0 or 1, p represents 0 or 1 and m represents an integer from 1 to 6;
R₂ represents a radical of formula —(CH₂)_{n′}[Q′]_{p′}[C(X′)(Y′)]_{m′}Z′;
   Q′ represents —O—;
   X′ represents the hydrogen atom;
   Y′ represents the hydrogen atom or phenyl;
   Z′ represents a hydrogen atom, (C₁-C₆)alkyl, amino, cyclohexyl, phenyl, naphthyl, fluorenyl, thienyl, furyl, benzothienyl, thiadiazolyl, indolyl, quinolyl, quinoxalyl, isoquinolyl, pyrazinyl, xanthenyl or naphthyridyl;
   the phenyl, naphthyl, quinolyl and thiadiazolyl radicals being optionally substituted by one or more identical or different substituents chosen from: —(CH₂)_{q}—X″—Y″, hydroxy, halo, nitro, (C₁-C₆)alkylamino, di((C₁-C₆)alkyl)amino;
   X″ represents —O—, —S— or a covalent bond;
   Y″ represents a (C₁-C₆)alkyl radical optionally substituted by one or more identical or different halo radicals, or phenyl radical optionally substituted by a halo radical;
   p′ represents 0 or 1; n′ represents an integer from 0 to 4; and m′ and q′ represent an integer from 0 to 4;
R₃ represents a radical of formula —(CH₂)_{n′}[Q′]_{p′}[C(X′)(Y′)]m′Z′;
   Q′ represents —C(O)—;
   X′ represents the hydrogen atom;
   Y′ represents the hydrogen atom, (C₁-C₆)alkyl, (C₁-C₆)alkoxy-carbonyl or phenyl;
   Z′ represents a hydrogen atom, (C₁-C₆)alkyl, (C₁-C₆)alkoxy-carbonyl, phenyl, naphthyl, fluorenyl, indolyl, benzothienyl or a radical of formula 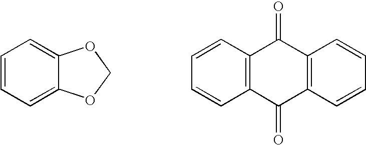
   the phenyl, benzothienyl and indolyl radicals being optionally substituted by one or more identical or different substituents chosen from: —(CH₂)q′-X″—Y″, (C₁-C₆)alkoxy, halo, nitro cyano, di((C₁-C₆)alkyl)amino;
   X″ represents —O—, —C(O)—, —C(O)—O—, —SO₂— or a covalent bond;
   Y″ represents a (C₁-C₆)alkyl radical optionally substituted by one or more identical or different halo radicals, or a phenyl radical;
   p′ represents 0 or 1; n′, m′ and q′ and m′ represent an integer from 0 to 6.

A more particular subject of the present invention is the compounds of general formula I as defined above in which
R₁ represents a radical of formula —(CH₂)_{n′}[Q′]_{p′}[C(X′)(Y′)]_{m′}Z′;
   X′ represents the hydrogen atom;
   Y′ represents the hydrogen atom or phenyl;
   Z′ represents a hydrogen atom, (C₁-C₆)alkyl, phenyl optionally substituted by one or more identical or different halo substituents, or naphthyl;
   p′ represents 0, n′ represents 0 or 1, and m′ represents an integer from 0 to 6;
R₂ represents the pyrrolydinyl radical or a radical of formula —(CH₂)ₙ—[Q]ₚ—(CH₂)ₘ—NXY
   Q represents —C(Z_{q})(Z_{q}′)—;
   Z_{q} represents the hydrogen atom and Z_{q}′ represents the hydrogen atom, phenyl optionally substituted by phenyl, cyclohexyl-methyl or benzyl;
   X and Y represent the hydrogen atom;
   p represents 0 or 1, and n represents 0 or 1, and m represents an integer from 0 to 6;
R₃ represents a radical of formula —(CH₂)_{n′}[Q′]_{p′}[C(X′)(Y′)]_{m′}Z′;
   X′ represents the hydrogen atom;
   Y′ represents the hydrogen atom, (C₁-C₆)alkyl, (C₁-C₆)alkoxy-carbonyl;
   Z′ represents a hydrogen atom, (C₁-C₆)alkyl, (C₁-C₆)alkoxy-carbonyl, phenyl, naphthyl or fluorenyl, or a radical of formula 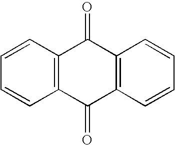
   the phenyl radical being optionally substituted by one or more identical or different substituents chosen from: —(CH₂)_{q′}X″Y″, halo, nitro, cyano;
   X″ represents —O—, —C(O)—, —C(O)—O— or a covalent bond;
   Y″ represents an alkyl radical optionally substituted by one or more identical or different halo radicals, or phenyl radical;
   p′ represents 0, n′ and m′ represent an integer from 0 to 6.

A more particular subject of the present invention is the compounds of general formula I as defined above in which
R₁ represents a radical of formula (CH₂)_{n′}[Q′]_{p′}[C(X′)(Y′)]_{m′}Z′;
   X′ represents a hydrogen atom;
   Y′ represents a hydrogen atom or phenyl;
   Z′ represents a hydrogen atom, (C₁-C₆)alkyl, naphthyl, or phenyl optionally substituted by one or more identical or different substituents chosen from: halo, amino or a phenyl;
   p′ represents 0, n′ represents 0 or 1, and m′ represents an integer from 0 to 6;
R₂ represents a radical of formula —(CH₂)_{n′}[Q′]_{p′}[C(X′)(Y′)]_{m′}Z′;
   X′ and Y′ represent a hydrogen atom;
   Z′ represents a hydrogen atom, (C₁-C₆)alkyl, phenyl, naphthyl, pyridine or benzothienyl,
   the phenyl radical being optionally substituted by one or more identical or different substituents chosen from: —(CH₂)_{q′}—X″—Y″;
   X″ represents —O— or a covalent bond;
   Y″ represents a (C₁-C₆)alkyl radical optionally substituted by one or more identical or different halo radicals, or phenyl radical;
   p′ represents 0, n′ represents 0 or 1, and m′ represents an integer from 0 to 6;
R₃ represents the piperidine ring or a radical of formula —(CH₂)ₙ—[Q]ₚ—(CH₂)ₘ—NXY
   Q represents —C(O)—NH—;
   X represents the hydrogen atom or (C₁-C₆)alkyl;
   Y represents the hydrogen atom, a (C₁-C₆)alkyl, or (pyridine)-ethyl, or X and Y form together with the nitrogen atom on which they are attached, the piperazine ring optionally substituted by a (C₁-C₆)alkyl;
   p represents 0 or 1, and n represents 0 or 1 and m represents an integer from 0 to 6.

A subject of the invention is also preferably the products of general formula I as defined above, characterized in that one of the R₁ or R₃ radicals represents a radical of formula —(CH₂)ₙ—[Q]ₚ—(CH₂)ₘ—NXY or —(CH₂)ₙ—W in which
W represents a heterocycloalkyl containing at least one nitrogen atom;
Q represents —O—, —S—, —C(O)—NH—, —C(Z_{q})(Z_{q}′)—, aryl or (C₃-C₇)cycloalkyl;
Z_{q} and Z_{q}′ represent, independently, the hydrogen atom, aryl optionally substituted by aryl, (C₃-C₇)cycloalkyl-alkyl, aralkyl, —C(O)O—R or —C(O)—NH—R′;
R represents a (C₁-C₆)alkyl, aryl or aralkyl radical, aryl and aralkyl being optionally substituted by one or more identical or different substituents chosen from: (C₁-C₆)alkoxy, hydroxy, halo, nitro cyano, amino, (C₁-C₆)alkylamino and di((C₁-C₆)alkyl)amino;
R′ represents a (C₁-C₆)alkyl, aryl, aralkyl, heteroaryl or heteroaryl-alkyl radical, the aryl, aralkyl, heteroaryl and heteroaryl-alkyl radicals being optionally substituted by one or more identical or different substituents chosen from: (C₁-C₆)alkoxy, hydroxy, halo, nitro cyano, amino, (C₁-C₆)alkylamino, di((C₁-C₆)alkyl)amino;
X and Y represent, independently, the hydrogen atom, (C₁-C₆)alkyl, (C₁-C₆)alkoxy-carbonyl or a heteroaryl-alkyl, or X and Y form together with the nitrogen atom on which they are attached, a heterocycloalkyl optionally substituted by a (C₁-C₆)alkyl;
p represents 0 or 1; n and m represent independently an integer from 0 to 6;
   and more preferentially,
R₁ represents a radical of formula —(CH₂)ₙ—[Q]ₚ—(CH₂)ₘ—NXY in which
   Q represents aryl or (C₃-C₇)cycloalkyl;
   X and Y represent, independently, the hydrogen atom, a (C₁-C₆)alkyl, or X and Y form together with the nitrogen atom on which they are attached, a heterocycloalkyl optionally substituted by a (C₁-C₆)alkyl;
   p represents 0 or 1, and n and m represent, independently, an integer from 0 to 6; or
R₃ represents a radical of formula —(CH₂)ₙ—[Q]ₚ—(CH₂)ₘ—NXY or —(CH₂)ₙ—W in which
   W represents a heterocycloalkyl containing at least one nitrogen atom;
   Q represents —C(O)—NH—;
   X and Y represent, independently, the hydrogen atom, (C₁-C₆)alkyl or a heteroaryl-alkyl, or X and Y form together with the nitrogen atom on which they are attached, a heterocycloalkyl optionally substituted by (C₁-C₆)alkyl;
   p represents 0 or 1, and n represents 0 or 1 and m represents an integer from 0 to 6.

A subject of the invention is also preferably the products of general formula I as defined above, characterized in that R₂ represents a radical of formula —(CH₂)_{n′}[Q′]_{p′}[C(X′)(Y′)]_{m′}Z′ in which
Q′ represents —O—;
X′ represents the hydrogen atom,
Y′ and Z′ represent, independently, a hydrogen atom, (C₁-C₆)alkyl, cyano, amino, (C₃-C₇)cycloalkyl, aryl or heteroaryl;
the aryl and heteroaryl radicals being optionally substituted by one or more identical or different substituents chosen from: —(CH₂)_{q′}—X″—Y″, hydroxy, halo, nitro, amino, (C₁-C₆)alkylamino, di((C₁-C₆)alkyl)amino;
X″ represents —O—, —S— or a covalent bond;
Y″ represents a (C₁-C₆)alkyl radical optionally substituted by one or more identical or different halo radicals, or aryl radical optionally substituted by one or more identical or different halo radicals;
p′ represents 0 or 1; n′ represents 0, 1 or 2; and m′ represents an integer from 0 to 6.

Also very preferentially, R₁ represents a radical of formula —(CH₂)ₙ—[Q]ₚ—(CH₂)ₘ—NXY in which
X and Y represent, independently, the hydrogen atom or a (C₁-C₆)alkyl;
p and n represent 0, and m represents an integer from 2 to 6.

Very preferentially, R₂ represents an optionally substituted aryl or heteroaryl radical and more particularly naphthyl, phenyl, benzothienyl, quinoxalyl, quinolyl, isoquinolyl or indolyl; the phenyl and naphthyl and quinolyl radicals being optionally substituted by one or more identical or different (C₁-C₆)alkoxy, halo, nitro, hydroxy, (C₁-C₆)alkyl radicals, the (C₁-C₆)alkyl itself being optionally substituted by one or more identical or different halo radicals.

Very preferentially, R₃ represents a radical of formula (CH₂)_{n′}[Q′]_{p′}[C(X′)(Y′)]_{m′}Z′ in which
X′ and Y′ represent the hydrogen atom;
Z′ represents indolyl or benzothienyl, the indolyl radical being optionally substituted by one or more identical or different substituents chosen from: —(CH₂)_{q}—X″—Y″, (C₁-C₆)alkoxy or halo, X″ represents —SO₂— or a covalent bond;
Y″ represents phenyl or alkyl optionally substituted by one or more identical or different halo radicals;
q′ represents 0 or 1; p′ represents 0; n′ represents 0 or 1; and m′ represents 0 or 1.

A subject of the present invention is also more particularly the compounds of general formula I as defined above in which
R₁ represents a radical of formula —(CH₂)ₙ[Q]ₚ[(CH₂)ₘ—NXY;
   Q represents the cyclohexyl radical;
   X and Y represent, independently, the hydrogen atom, a (C₁-C₆)alkyl, or X and Y form, together with the nitrogen atom on which they are attached, the piperidine ring;
   n represents 0 or 1, p represents 0 or 1 and m represents an integer from 1 to 6;
R₂ represents a radical of formula —(CH₂)_{n′}[Q′]_{p′}[C(X′)(Y′)]_{m′}Z′;
   Q′ represents —O—;
   X′ represents the hydrogen atom;
   Y′ represents the hydrogen atom or phenyl;
   Z′ represents a hydrogen atom, (C₁-C₆)alkyl, amino, cyclohexyl, phenyl, naphthyl, fluorenyl, thienyl, furyl, benzothienyl, thiadiazole, indolyl, quinolyl, quinoxalyl, isoquinolyl, pyrazinyl, xanthenyl or naphthhyridyl; the phenyl, naphthyl, quinolyl and thiadiazolyl radicals being optionally substituted by one or more identical or different substituents chosen from: (CH₂)_{q′}—X″—Y″ hydroxy, halo, nitro, (C₁-C₆)alkylamino, di((C₁-C₆)alkyl)amino;
   X″ represents —O—, —S— or a covalent bond;
   Y″ represents a (C₁-C₆)alkyl radical optionally substituted by one or more identical or different halo radicals, or phenyl optionally substituted by a halo radical;
   p′ represents 0 or 1; n′ represents 0, 1 or 2; and m′ represents an integer from 0 to 4;
R₃ represents a radical of formula —(CH₂)_{n′}[Q′]_{p′}[C(X′)(Y′)]_{m′}Z′;
   Q′ represents —C(O)—;
   X′ represents the hydrogen atom;
   Y′ represents the hydrogen atom, (C₁-C₆)alkyl, (C₁-C₆)alkoxy-carbonyl or phenyl;
   Z′ represents a hydrogen atom, (C₁-C₆)alkyl, (C₁-C₆)alkoxy-carbonyl, phenyl, naphthyl, fluorenyl, indolyl, benzothienyl, or a radical of formula 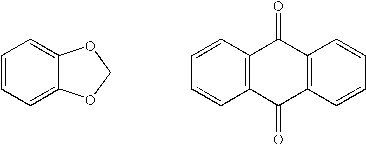
   the phenyl, benzothienyl and indolyl radicals being optionally substituted by one or more identical or different substituents chosen from: (CH₂)_{q′}X″Y″, (C₁-C₆)alkoxy, halo, nitro, cyano, di((C₁-C₆)alkyl)amino;
   X″ represents —O—, —C(O)—, —C(O)—O—, —SO₂— or a covalent bond;
   Y″ represents a (C₁-C₆)alkyl radical optionally substituted by one or more identical or different halo radicals, or a phenyl radical;
   p′ represents 0 or 1; n′ represents 0, 1 or 2; and m′ represents an integer from 0 to 6.
      and very preferentially
R₁ represents a radical of formula —(CH₂)ₙ—[Q]ₚ—(CH₂)ₘ—NXY in which
   X and Y represent, independently, the hydrogen atom or a (C₁-C₆)alkyl;
   p and n represent 0, and m represents an integer from 2 to 6.
R₂ represents quinoxalyl, quinolyl or naphthyl, the quinolyl and naphthyl radicals being optionally substituted by one or more identical or different (C₁-C₆)alkyl, (C₁-C₆)alkoxy, halo radicals;
R₃ represents a radical of formula —(CH₂)_{n′}[Q′]_{p′}[C(X′)(Y′)]_{m′}Z′ in which
   X′ and Y′ represent the hydrogen atom;
   Z′ represents indolyl optionally substituted by one or more identical or different substituents chosen from: —(CH₂)_{q}—X″—Y″, (C₁-C₆)alkoxy or halo;
   X″ represents a covalent bond;
   Y″ represents an alkyl radical optionally substituted by one or more identical or different halo radicals;
   q′ represents 0 or 1; p′ represents 0; n′ represents 0 or 1; and m′ represents 0 or 1

In the present Application, the symbol ->* corresponds to the point of attachment of the radical. When the attachment site is not specified on the radical, that signifies that the attachment is carried out on one of the sites available for such an attachment of this radical.

The compounds according to the invention can be prepared in liquid phase according to the following general diagram: 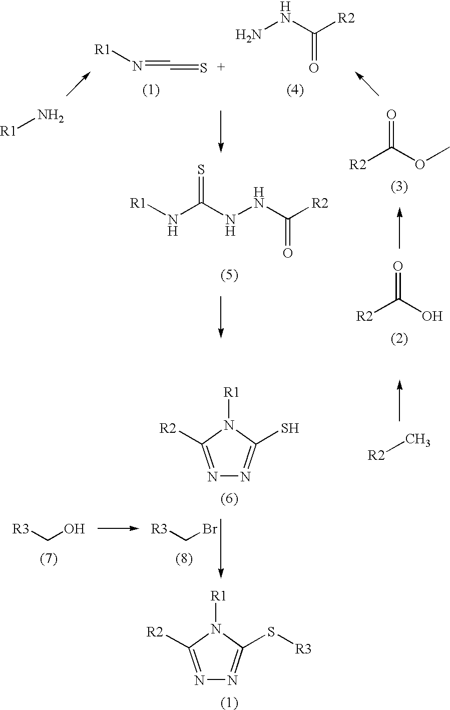
1. Preparation of Isothiocyanates (1):

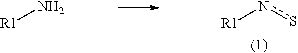

The isothiocyanates of general formula (1) can be prepared from the corresponding primary amines by two methods:

1.1 Method A:

A primary amine is converted to isothiocyanate by the action of O,O-di(2-pyridinyl) thiocarbonate (1 eq.) in anhydrous aprotic solvents such as dichloromethane, tetrahydrofuran or dimethylformamide (Kim, S.; Lee, J. I. Tetrahedron Lett. 1985, 26 (13), 1661-1664). The reaction mixture is stirred at ambient temperature for 1 to 4 hours then the solvents are evaporated off and the residue used in the following stage without other purification.

### Preparation 1: tert-butyl 4-isothiocyanatobutylcarbamate (C₁₀H₁₈N₂O₂S, M=230.33)

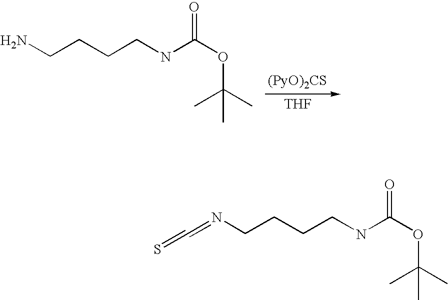

Tert-butyl 4-aminobutylcarbamate (3.2 ml; 17 mmol) is added to O,O-di(2-pyridinyl) thiocarbonate (3.9 g; 17 mmol) dissolved in tetrahydrofuran. The solution is stirred for 2 hours at ambient temperature. The solvent is evaporated off and the solid obtained is used without delay in the following stage.

The isothiocyanates of general formula R₁NCS were synthesized according to this operating process with the following R₁ groups the primary and secondary amines of which are protected by a tert-butoxycarbonyl group:

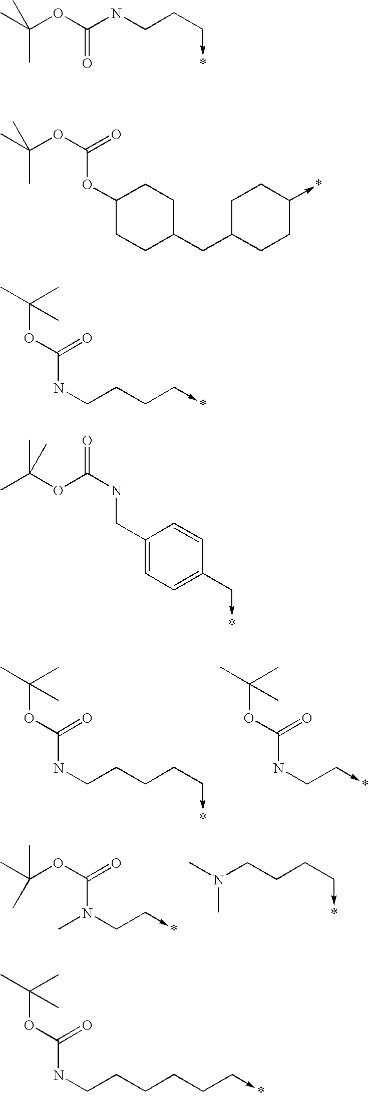

1.2 Method B:

A primary amine is converted to isothiocyanate by the action of dithioxomethane (10 eq.) in the presence of N-cyclohexylcarbodiimide, N-methylpolystyrene resin (Novabiochem; load greater than 1.5 mmol/g, 1.1 eq.) pre-swollen in an aprotic solvent such as dichloromethane or tetrahydrofuran. The reaction mixture is stirred at ambient temperature for 1 to 4 hours, then the filtrate is evaporated and used in the following stage without other purification.

### Preparation 2: N,N-dimethyl-4-isothiocyanatobutylamine (C₇H₁₄N₂S, M=158.27)

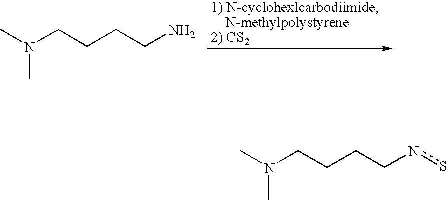

Dithioxomethane (1 ml; 16.6 mmol) is added to N-cyclohexylcarbodiimide, N-methylpolystyrene resin (1 g; 1.69 mmol/g; Novabiochem) in dicholoromethane (15 ml). The suspension is stirred for 30 minutes then N,N-dimethyl-1,4-butanediamine (0.19 ml; 1.5 mmol) is added. The reaction mixture is stirred for 3 hours, then filtered. The filtrate is evaporated and used immediately in the following stage.

The isothiocyanates of general formula R₁NCS were synthesized according to this method with the following R₁ groups the primary amines of which are protected by a tert-butoxycarbonyl group: 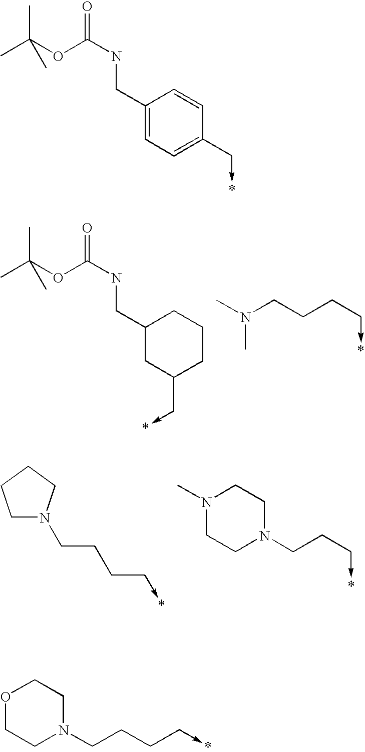
2. Preparation of Hydrazides (4):

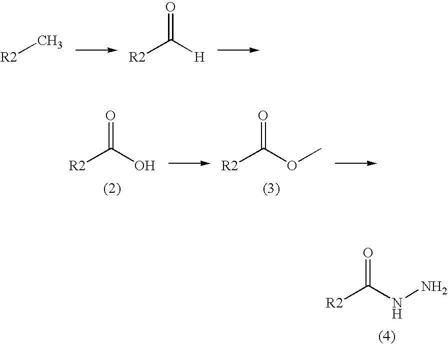

2.1 Preparation of Carboxylic Acids (2):

When they are not commercially available, the carboxylic acids of general formula (2), in which R₂ is a group of aryl or heteroaryl type, can be prepared from the corresponding methylated derivative by oxidation to aldehyde, for example with selenium dioxide, followed by a second oxidation to carboxylic acid, using, for example, sodium chlorite (Bu, X.; Deady, L. W.; Finlay, G. J.; Baguley, B. C.; Denny, W. A. J. Med. Chem. 2001, 44, 2004-2014).

### Preparation 3: 6-chloroquinoline-2-carboxylic Acid (C₁₀H₆ClNO₂, M=207.62)

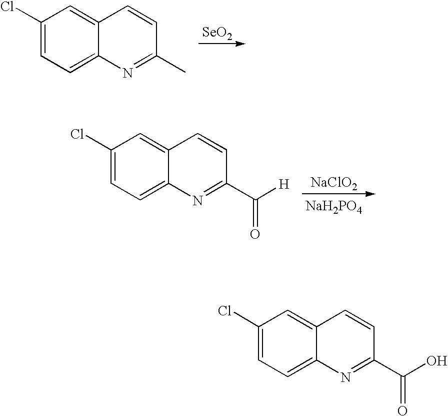

6-chloro-2-methylquinoline (500 mg; 2.8 mmol) is added to a suspension of selenium dioxide (1.87 g; 16.9 mmol; 6 eq.) in dioxane (25 ml) at 80° C. The reaction mixture is stirred for 3 hours under reflux then the insoluble matter is filtered while warm. The dioxane is then evaporated off under reduced pressure and the aldehyde obtained is used without purification in the following stage.

NMR ¹H (DMSO-d₆, 400 MHz) δ: 10.09 (s, 1H, CHO); 8.57-8.54 (m, 1H, arom. H); 8.27-8.21 (m, 2H, arom. H); 8.02-8.00 (m, 1H, arom. H); 7.91-7.88 (m, 1H, arom. H).

A solution of sodium chlorite (2.4 g) and sodium dihydrogen phosphate (2.4 g) in water (24 ml) is added, over a period of 5 minutes, to a solution of 6-chloroquinoline-2-carbaldehyde (536 mg; 2.8 mmol) in ter-butyl alcohol (56 ml) and 2-methylbut-2-ene (14 ml). The mixture obtained is stirred for 4 hours at ambient temperature. The organic solvents are evaporated off under reduced pressure and water (30 ml) is added to the residue. The precipitate obtained is filtered, washed with water and dried under vacuum in the presence of P₂O₅. 6-chloroquinoline-2-carboxylic acid is obtained in the form of white powder (505 mg; yield=87%). MS/LC: m/z=208.01 (M+H) rt=8.55 min (condition 1).

NMR ¹H (DMSO-d₆, 400 MHz) δ: 8.41-8.39 (m, 1H, arom. H); 8.20-8.11 (m, 3H, arom. H); 7.82-7.79 (m, 1H, arom. H).

Carboxylic acids of general formula R₂COOH were synthesized according to this method with the following R₂ groups:

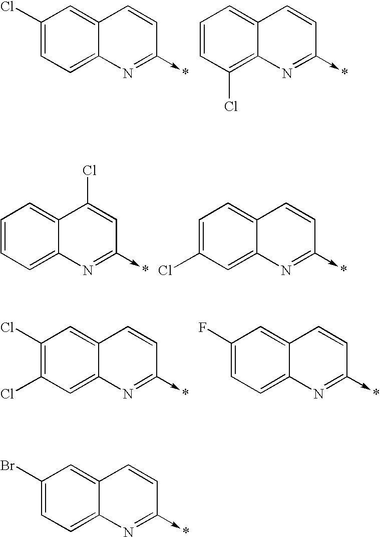

2.2 Preparation of Methyl Esters (3):

A carboxylic acid is firstly converted to methyl ester for example by the action of an excess of diazomethane or a diazomethane substitute such as trimethylsilyldiazomethane, in methanol, in the presence or not of an aprotic solvent such as diethyl ether or dichloromethane (Caturla, F.; Najera, C.; Varea, M. Tetrahedron Lett. 1999, 40 (32), 5957-5960). The excess of diazomethane is neutralised by the addition of a carboxylic acid such as for example acetic acid. The methyl esters are isolated after extraction and washing and used without other purification in the following stage.

### Preparation 4: methyl 4-fluoro-1-naphthoate (C₁₂H₉FO₂, M=204.20)

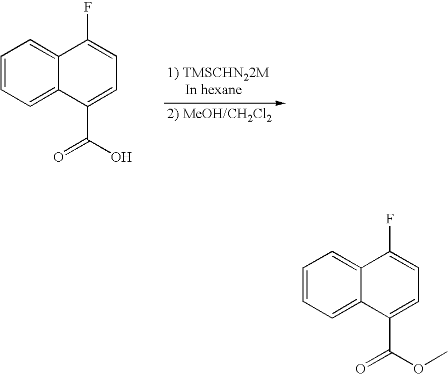

A solution of (trimethylsilyl)diazomethane in solution in hexane (6 ml, 2 mol/l) is added to 4-fluoro-1-naphthoic acid (1 g; 5.3 mmol) dissolved in a mixture of dichloromethane (10 ml) and methanol (15 ml) until the solution retains a slight yellow coloration and no longer degases. The excess of (trimethylsilyl)diazomethane is neutralised by the addition of a few drops of acetic acid until the solution is colourless. The reaction mixture is evaporated, followed by solubilizing in ethyl acetate (20 ml) and washing with distilled water (10 ml), then a saturated solution of sodium chloride (10 ml). The organic phase is dried over sodium sulphate, followed by evaporating and drying under vacuum in order to produce a white powder (0.78 g; yield=73%). MS/LC: m/z=205.23 (M+H) rt=11.21 min (condition 1).

Methyl esters of formula R₂COOMe were synthesized with the following R₂ groups the primary and secondary amines of which are protected by a tert-butoxycarbonyl group:

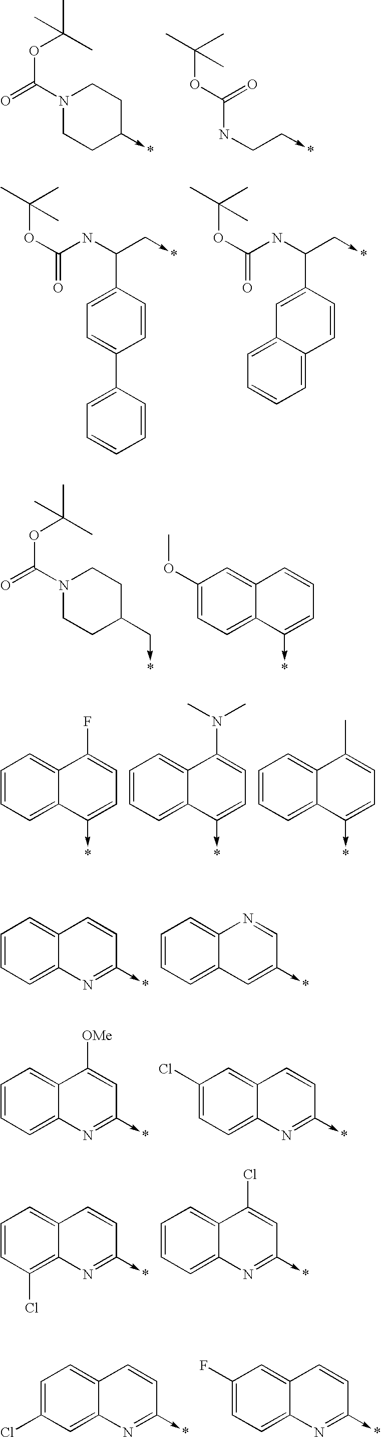

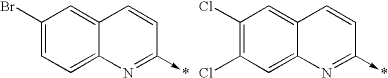

2.3 Preparation of the Hydrazides (4):

The hydrazides of general formula (4) can be obtained by the action of hydrazine hydrate (3 to 10 eq.) on the esters of general formula (3) in a protic polar solvent such as ethanol or methanol (Leung, H. K.; Phillips, B. A.; Cromwell, N. H., J. Heterocycl. Chem. 1976, 13, 247-252). The reaction is maintained for 18 to 96 hours at ambient temperature or at 50° C. After evaporation, the reaction medium is taken up in a solvent such as ethyl acetate followed by washing with water. The hydrazides are obtained after evaporation of the organic phases and solidification.

### Preparation 5: 2,2-diphenylacetohydrazide (C₁₄H₁₄N₂O, M=226.28)

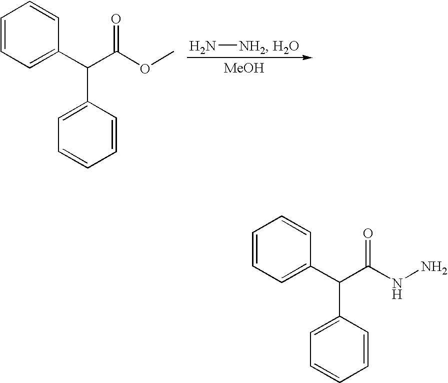

Hydrazine hydrate (7 ml; 50 mmol) is added to methyl diphenylacetate (1.19 g; 5 mmol) solubilized in methanol (15 ml). The reaction mixture is stirred at ambient temperature for 60 hours then the solvent is evaporated off. The residue is solubilized in ethyl acetate (20 ml) followed by washing with distilled water (15 ml) then a saturated aqueous solution of sodium chloride (15 ml). The organic phase is dried over magnesium sulphate, followed by evaporating and drying under vacuum in order to produce a white powder (0.94 g; yield=83%).

NMR ¹H (DMSO-d₆, 400 MHz) δ: 9.44 (broad s, 1H, NH); 7.33-7.20 (m, 10H, arom.); 4.82 (s, 1H, CH); 4.30 (broad s, 2H, NH₂). MS/LC: m/z=227.30 (M+H) rt=10.19 min (condition 1).

The hydrazides of formula R₂CONHNH₂ were prepared with the following R₂ groups, the primary and secondary amines of which are protected by a tert-butoxycarbonyl group: 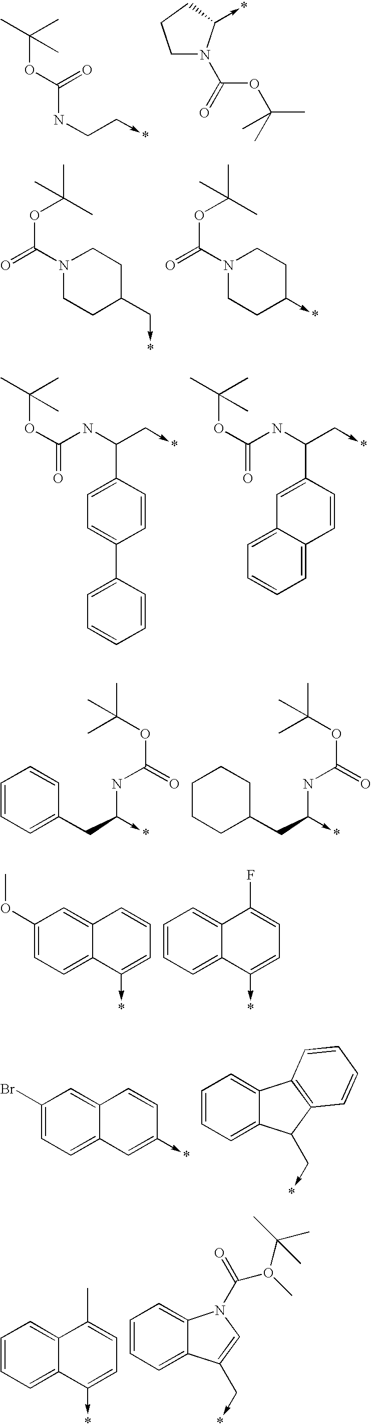 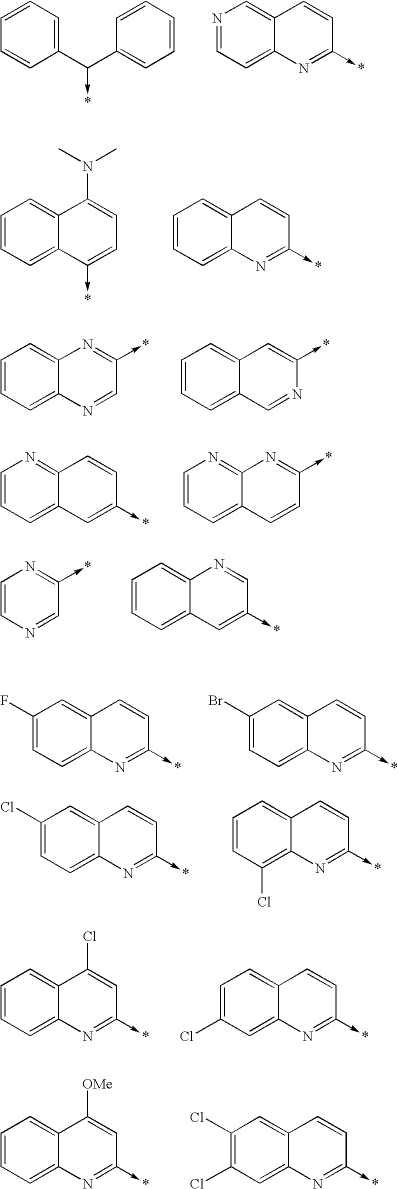
3. Preparation of Hydrazinecarbothioamides (5):

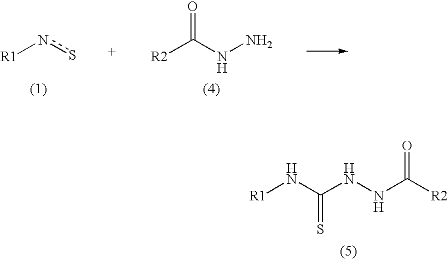

The isothiocyanates of general formula (1) (1.1 eq.) are added to the hydrazides of general formula (4) in an aprotic solvent such as dichloromethane or dimethylformamide and the reaction medium is stirred at ambient temperature for 18 to 24 hours. The hydrazinecarbothioamides (5) are obtained after filtration or evaporation of the reaction medium and used in the following stage without other purification.

### Preparation 5: N-phenyl-2-(phenylacetyl)hydrazinecarbothioamide (C₁₅H₁₅N₃OS, M=285.37)

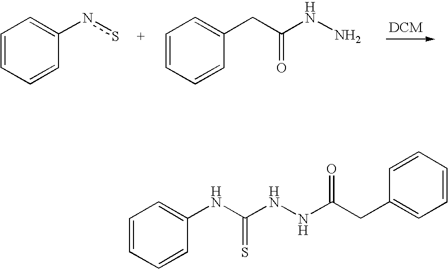

Phenylisothiocyanate (1.3 ml; 11 mmol) is added to 2-phenylacetohydrazide (1.5 g; 10 mmol) solubilized in dichloromethane (20 ml). The solution is stirred at ambient temperature until precipitation of the product. The white solid formed is filtered, followed by washing with ethyl ether (10 ml) and drying under vacuum (2.1 g; yield=74%).

NMR ¹H (DMSO-d₆, 400 MHz) δ: 10.15 (broad s, 1H, NH); 9.59 (broad s, 2H, NH₂); 7.44-7.42 (m, 2H, arom.); 7.35-7.28 (m, 5H, arom.); 7.25-7.22 (m, 2H, arom.); 7.18-7.14 (m, 1H, arom.); 3.52 (s, 2H, CH₂). MS/LC: m/z=286.26 (M+H) rt=8.13 min (condition 1).

The hydrazinecarbothioamides of general formula (5) were synthesized for the preparation of the compounds of the invention with the following R₁ and R₂ groups the primary and secondary amines of which are protected by a tert-butoxycarbonyl group: 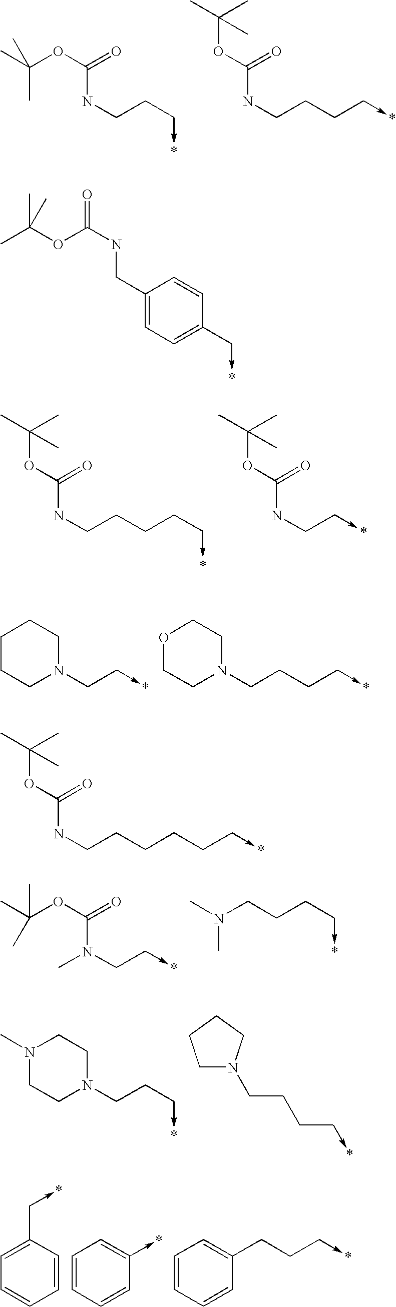 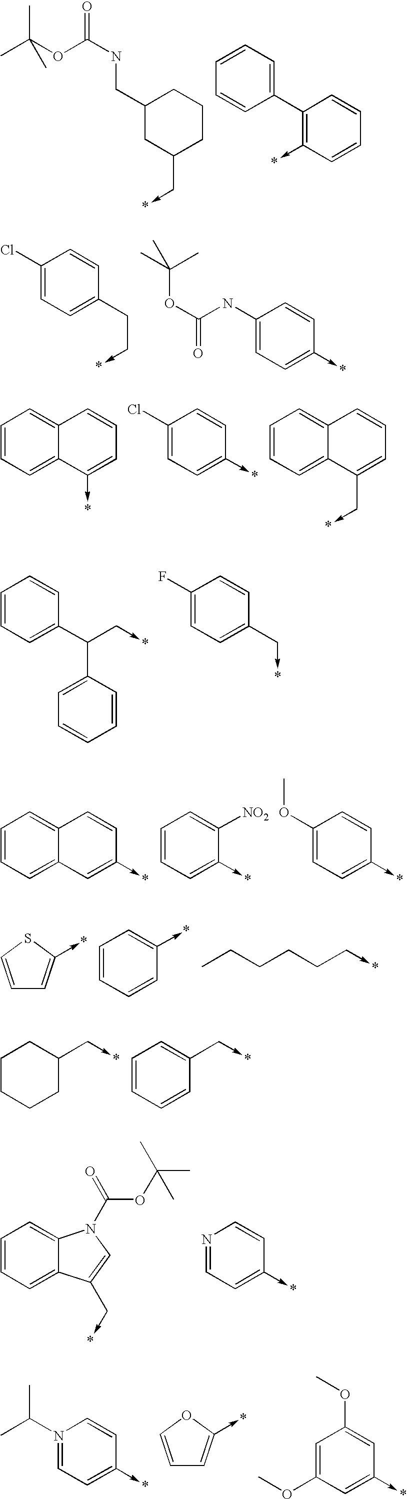 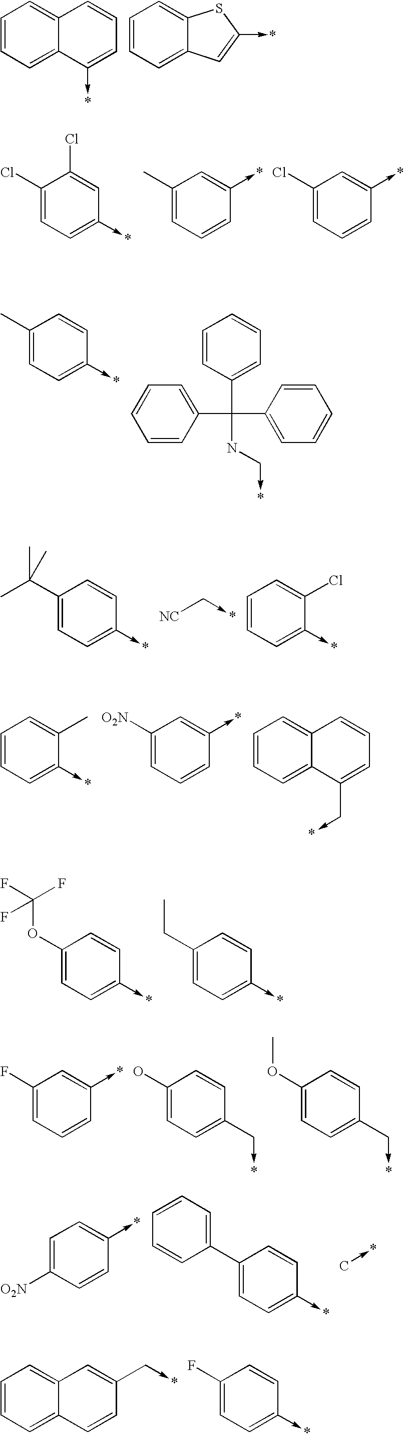 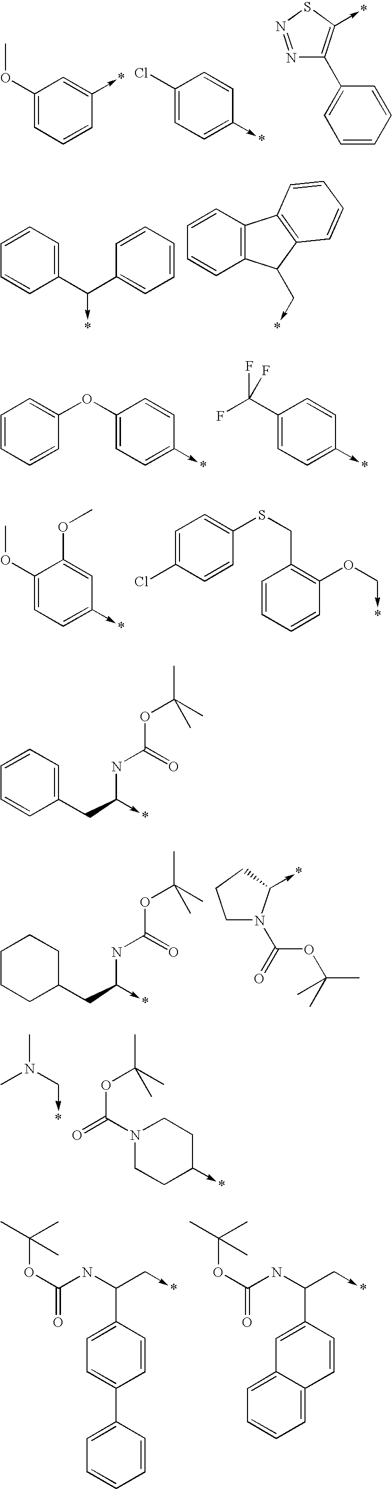 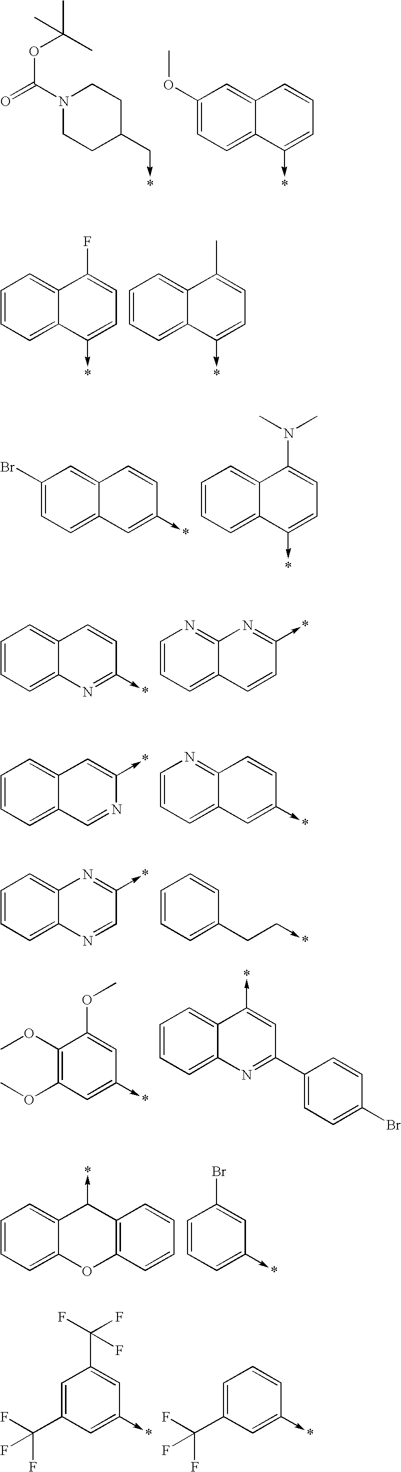 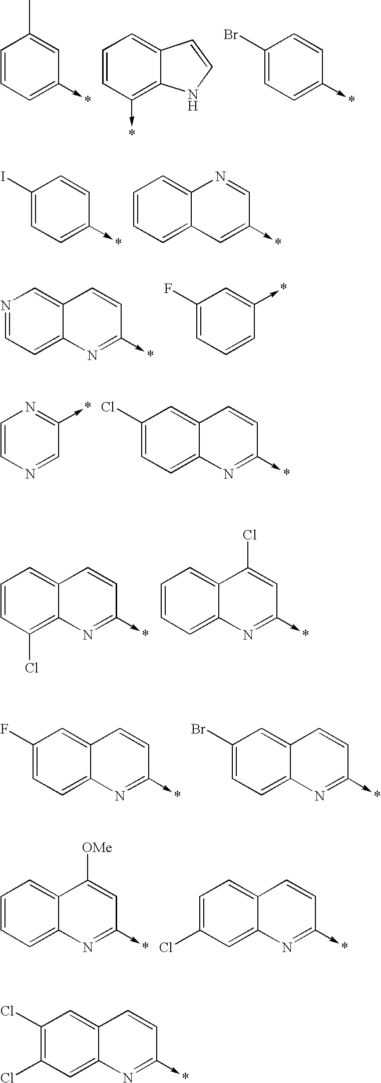
4. Preparation of Triazoles (6):

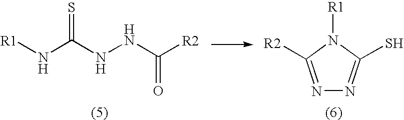

After dissolution of the hydrazinethiocarbamide of general formula (5) in a solvent of dioxane or toluene type, the cyclization stage takes place in a protic solvent such as ethanol or methanol in the presence of a solution of soda (1M to 4M) or of potash (1M to 4M). The reaction is maintained at 85° C. over a period ranging from 4 hours to 18 hours then after evaporation of the solvents, the thiolate obtained is converted to thiol (6) for example using an ion exchange resin such as Amberlite resin IRN 77 (H+cation) (Prolabo). The resin is filtered and the filtrate concentrated. Purification on a silica column can be carried out.

### Preparation 7: 5-benzyl-4-phenyl-4H-1,2,4-triazole-3-thiol (C₁₅H₁₃N₃S, M=267.35)

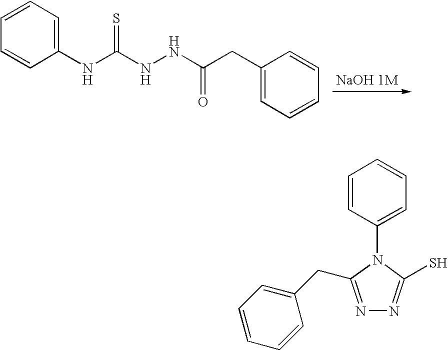

A normal aqueous solution of sodium hydroxide (20 ml) is added to N-phenyl-2-(phenylacetyl)hydrazinecarbothioamide (3.7 g; 13 mmol) dissolved in a mixture of dioxane (30 ml) and methanol (10 ml). The solution is stirred and heated at 85° C. for 4 hours. The solvents are evaporated off and the residue is solubilized in methanol (25 ml). An ion exchange resin pre-rinsed with the methanol (Amberlite IRN 77, 50 g, Prolabo) is added to the solution, followed by stirring for 15 minutes then filtering. The filtrate is evaporated then dried under vacuum (3.4 g; yield=98%.

NMR ¹H (DMSO-d₆, 400 MHz) δ: 13.78 (broad s, 1H, SH); 7.48-7.46 (m, 3H, arom.); 7.23-7.17 (m, 5H, arom.); 6.92-6.90 (m, 2H, arom.); 3.85 (s, 2H, CH₂). MS/LC: m/z=268.23 (M+H) rt=5.72 min (condition 2).

The triazoles of general formula (6) were prepared with the same groups R₁ and R₂ as those described for the preparation of the hydrazinecarbothioamides (5) above.

5. Preparation of Brominated Intermediates (8):

5.1 Preparation of Benzyl Bromides (8a):

5.1.1 General Case:

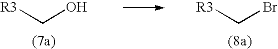

The benzyl bromides of general formula (8a) can be obtained from the corresponding alcohols (7a) according to the procedures described in literature, for example by treatment with aqueous hydrobromic acid under reflux (Kinoshita, T.; Okunaka, T.; Ohwada, H.; Furukawa, S. J. Heterocycl. Chem. 1991, 28 (8), 1901-1909) or with an inorganic acid halide such as PBr₃ or SOBr₂ (Nagle, A. S.; Salvatore, R. N.; Chong, B.-D.; Jung, K. W. Tetrahedron Lett 2000, 41 (17), 3011-3014) or also with a mixture of N-bromosuccinimide or CBr₄ and triphenylphosphine in an aprotic solvent such as tetrahydrofuran or dichloromethane (Amici, R.; Pevarello, P.; Colombo, M.; Varasi, M. Synthesis 1996, (10), 1177-1179, Campbell, J. A.; Rapoport, H. J. Org. Chem. 1996, 61 (18), 6313-6325).

### Preparation 8: 5-(bromomethyl)-1,3-benzodioxole (C₈H₇BrO₂, M=215.05)

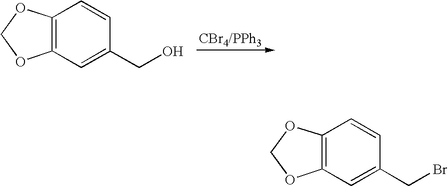

Carbon tetrabromide (3.8 g; 11.5 mmol) is added to 5-(methanol)-1,3-benzodioxole (1.5 g; 10 mmol) dissolved in dicholoromethane (30 ml); the mixture is cooled down to 0° C. Triphenylphosphine (3.0 g; 11.5 mmol) is added in portions, the solution is stirred for two hours at ambient temperature. The solvent is evaporated off and the solid obtained is purified by chromatography on a silica column (eluent: heptane/ethyl acetate: 3/1). The fractions are evaporated and the solid obtained is dried under vacuum (2.1 g; yield=97%).

NMR ¹H (DMSO-d₆, 400 MHz) δ: 7.02-6.98 (m, 1H, arom.); 6.96-6.93 (m, 1H, arom); 6.88-6.86 (m, 1H, arom); 6.02 (s, 2H, CH₂); 4.66 (s, 2H, CH₂).

A benzyl bromide of formula R₃Br was synthesized with the following R₃ group:

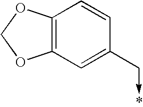

5.1.2 The Particular Case of the Methylindole Bromides (8a):

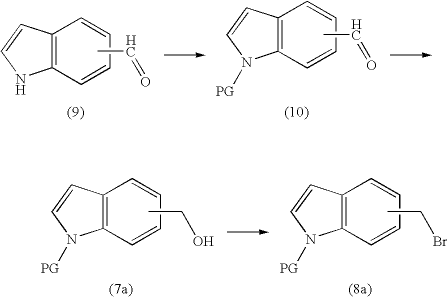

In the case where the brominated intermediates of general formula (8a) are of methylindole type, they can be obtained in 3 stages from the corresponding indole-carbaldehydes, first by protection of the indole, then by reduction of the aldehyde function followed finally by bromination of the alcohol function thus obtained.

5.1.2.1 Protection of the Indole:

A suitable protective group such as, for example, a group of carbamate type (for example, the tert-butoxycarbonyl group) is introduced onto the indole by standard methods known to a person skilled in the art (P. J. Kocienski, Protecting Groups, 192 (Georg Thiem Verlag Stuttgart, 1994)), for example using di-tert-butyl dicarbonate in acetonitrile or dimethylformamide at ambient temperature in the presence of a catalyst such as dimethylaminopyridine.

### Preparation 9: tert-butyl 6-formyl-1H-indole-1-carboxylate (C₁₄H₁₅NO₃, M=245.28)

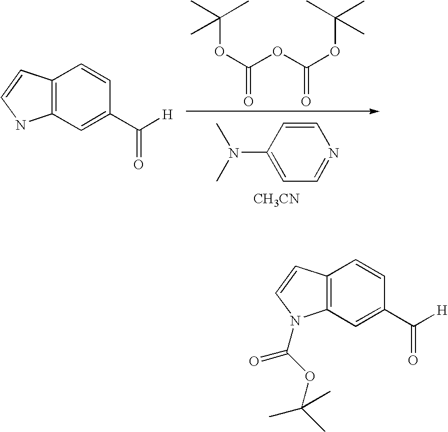

Di-tert-butyl dicarbonate (0.827 g; 3.8 mmol) and 4-N-dimethylaminopyridine (0.19 mmol; 21 mg) are added to 1H-indole-6-carbaldehyde (0.5 g; 3.44 mmol) dissolved in acetonitrile (15 ml). The mixture is stirred at ambient temperature for 16 hours. The acetonitrile is evaporated off; the residue dissolved in ethyl acetate (30 ml) is washed twice with distilled water (20 ml) then with a saturated aqueous solution of sodium chloride (20 ml). The organic phase is dried over magnesium sulphate followed by evaporating and drying under vacuum. The expected product is obtained in the form of a white solid (0.514 g; yield=61%).

NMR ¹H (DMSO-d₆, 400 MHz) δ: 10.06 (s, 1H, CHO); 8.60 (s, 1H, arom.); 7.92-7.91 (d, J=3.7 Hz, 1H, arom.), 7.81-7.75 (m, 2H, arom.); 6.84-6.83 (d, J=3.7 Hz, 1H, arom.); 1.65 (s, 9H, t-Bu). MS/LC: m/z=268.23 (M+H) rt=5.72 min (condition 1).

5.1.2.2 Preparation of the Alcohols (7a):

The alcohols of general formula (7a) can be obtained by reducing aldehydes of general formula (10) by standard methods known to a person skilled in the art such as, for example, by the action of the system: NiCl₂.6H₂O—Zn in a water/DMF mixture at ambient temperature (Baruah, R. N. Tetrahedron Lett. 1992, 33 (37), 5417-5418) or by using NaBH₄ in ethanol at ambient temperature (Cho, Y. J.; Lee, S. H.; Bae, J. W.; Pyun, H. J.; Yoon, C. M. Tetrahedron Lett. 2000, 41 (20), 3915-3917) or also by using Bu₃SnH in a protic solvent such as, for example, methanol (Kamiura, K.; Wada, M. Tetrahedron Lett. 1999, 40 (51), 9059-9062).

### Preparation 10: tert-butyl 6-(hydroxymethyl)-1H-indole-1-carboxylate (C₁₄H]₇NO₃; M=247.30)

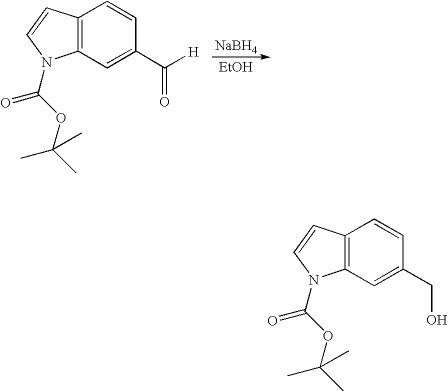

Tert-butyl 6-formyl-1H-indole-1-carboxylate (0.514 g: 2.1 mmol) is dissolved in ethanol (5 ml) then sodium borohydride (0.159 g; 4.2 mmol) is added slowly and the solution is stirred at ambient temperature for 2 hours. The solvent is evaporated off, and the residue redissolved in ethyl ether (20 ml) is washed with a solution of sodium hydroxide (1N; 10 ml) then with a saturated solution of sodium chloride (10 ml). The organic phase is dried over magnesium sulphate, followed by evaporating and drying under vacuum. The alcohol is obtained in the form of a white solid (0.48 g, yield=93 %).

NMR ¹H (DMSO-d₆, 400 MHz) δ: 8.69 (s, 11H, arom.); 7.61-7.60 (d, J=3.6 Hz, 1H, arom.); 7.54-7.52 (d, J=8 Hz, 1H, arom.); 7.18-7.16 (d, J=8 Hz, 1H, arom.); 6.66-6.65 (d, J=3.6 Hz, 1H, arom.); 5.22-5.19 (t, J=5.7 Hz, 1H, OH); 4.60-4.59 (d, J=5.7 Hz, 2H, CH₂); 1.62 (s, 9H, t-Bu). MS/LC: m/z=fragmented; rt=10.28 min (condition 1).

5.1.2.3 Preparation of the Methylindole Bromides (8a):

The methylindole bromides of general formula (8a) are obtained from the alcohols of general formula (7a) according to the general bromination processes described above for the preparation of benzyl bromides.

A methylindole bromide of formula R₃Br was synthesized with the following R₃ group:

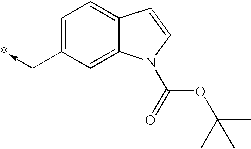

5.2 Preparation of the Ethylindole Bromides (8b):

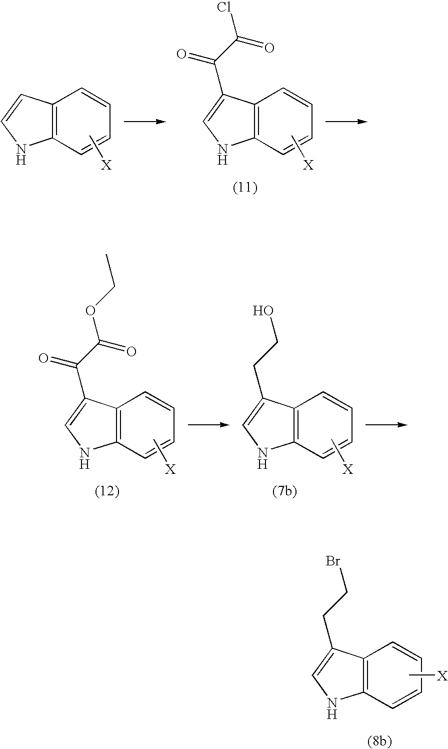

In the case where the brominated intermediates of general formula (8) are of ethylindole type, they can be obtained in 4 stages from the corresponding indoles, firstly by conversion to α-ketoacid chloride (11) followed by a conversion to α-ketoester (12) then a reduction to alcohol (7b), to finally prepare the brominated intermediate (8b).

5.2.1 Obtaining the α-ketoacid Chlorides (11):

The α-ketoacid chlorides (11) can be obtained by the action of oxalyl chloride in an apolar aprotic solvent such as, for example, diethylether, at ambient temperature (Woodward, R. B.; Bader, F. E.; Bickel, H.; Frey, A. J.; Kierstead, R. W. Tetrahedron 1952 2, 1).

### Preparation 11: (6-methoxy-1H-indol-3-yl)(oxo)acetyl Chloride (C₁₁H₈ClNO₃, M=237.64).

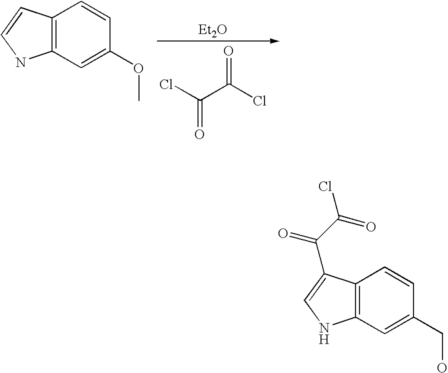

5-methoxyindole (1 g; 6.8 mmol) dissolved in ethyl ether (25 ml) is cooled down to 0° C. Oxalyl chloride (8.8 mmol; 0.77 ml) is added dropwise under argon and the mixture is stirred at ambient temperature under an argon atmosphere for three hours. The expected product is obtained in the form of a yellow powder after filtration and washing with ethyl ether.(1.44 g, yield=89%).

NMR ¹H (DMSO-d₆, 400 MHz) δ: 12.19 (s, 1H, NH); 8.27 (s, 1H, arom.); 8.01-7.99 (d, J=8.7 Hz, 1H, arom.); 7.02 (s, 1H, arom.); 6.90-6.87 (d, J=8.7 Hz, 1H, arom.); 3.79 (s, 1H, OCH₃).

The α-ketoacid chlorides of formula R₃—OC(O)C(O)Cl were prepared with the following indole R₃ groups:

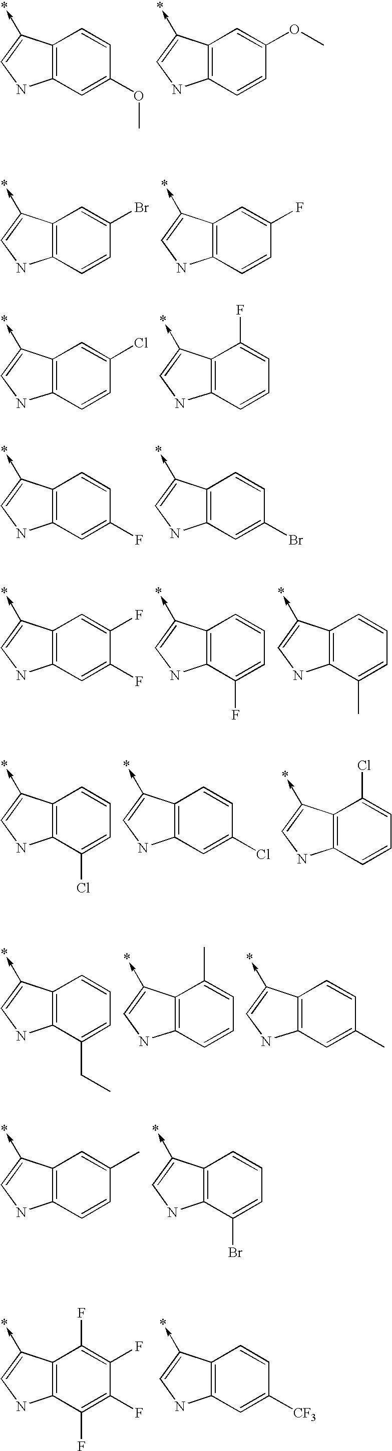

5.2.2 Esterification of the α-Ketoacid Chlorides (11) to α-Ketoesters (12):

The indole α-ketoesters (12) are obtained by standard esterification methods known to a person skilled in the art, such as, for example, treatment of the corresponding α-ketoester chloride with an alcohol (such as methanol or ethanol) in the presence of an organic base such as, for example, triethylamine or diisopropylethylamine.

### Preparation 12: ethyl (6-methoxy-1H-indol-3-yl)(oxo)acetate (C₁₃H₁₃NO₄, M=247.25)

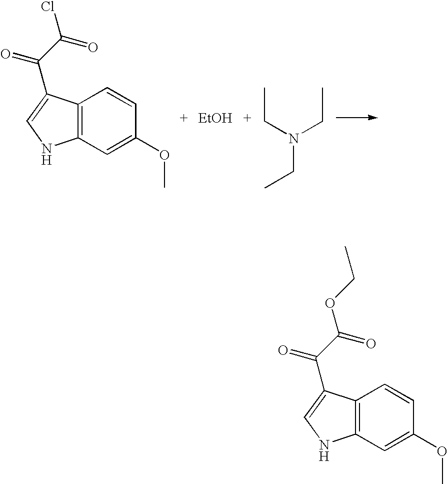

(6-methoxy-1H-indol-3-yl)(oxo)acetyl chloride (1.44 g; 6.06 mmol) dissolved in ethanol (15 ml) is cooled down to 0° C. then triethylamine (1.04 ml; 7.5 mmol) is added dropwise. The mixture is heated under reflux for 2 hours. The precipitate is filtered, followed by washing with ethanol (5 ml) and ethyl ether (5 ml) then drying under vacuum. The expected product is obtained in the form of a yellow powder (1.36 g; yield=91%).

NMR ¹H (DMSO-d₆, 400 MHz) δ: 12.16 (s, 1H, NH); 8.28 (s, 1H, arom.); 8.00-7.98 (d, J=8.6 Hz, 1H, arom.); 7.02 (s, 1H, arom.); 6.91-6.88 (d, J=8.6 Hz, 1H, arom.); 4.37-4.31 (q, J=7 Hz, 2H, OCH₂); 3.79 (s, 3H, OCH₃); 1.34-1.31 (t, J=7 Hz, 3H, CH₃).

The α-ketoesters of formula R_{3′}C(O)C(O)OEt were prepared with the following indole R_{3′} groups:

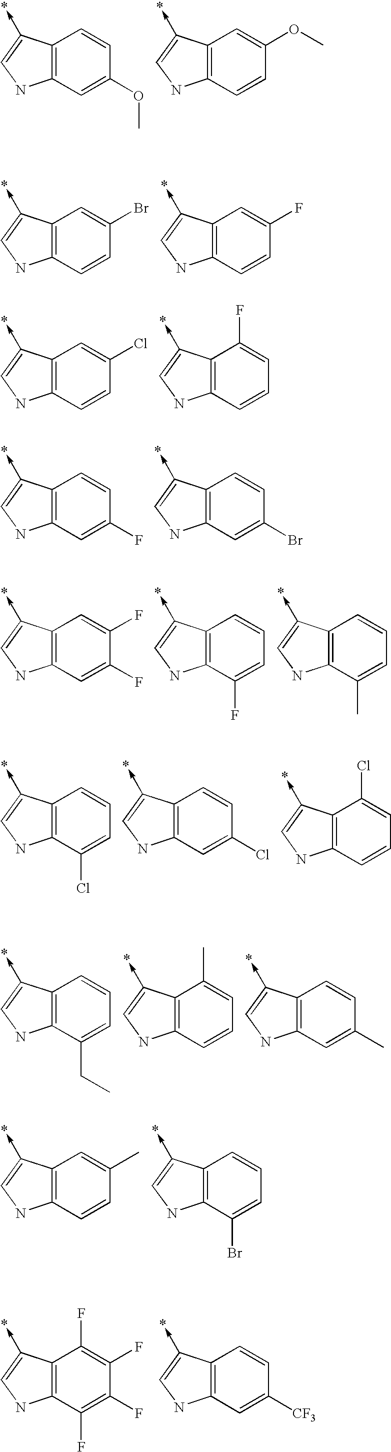

5.2.3 Reduction of α-Ketoesters (12) to Ethylindole Alcohols (7b):

The α-ketoesters of general formula (12) can be reduced to ethyl alcohols of general formula (7b) by treatment for example with lithium and aluminium hydride in an aprotic solvent such as tetrahydrofuran at reflux (Feldman, P. L.; Rapoport, H. Synthesis 1986 (9), 735-737).

### Preparation 13: 2-(6-methoxy-1H-indol-3-yl)ethanol (C₁₁H₁₃NO₂, M=191.23).

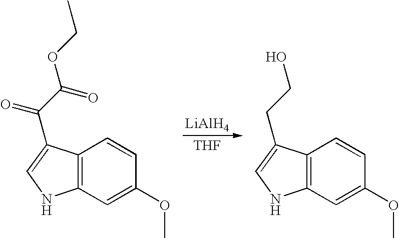

Ethyl (6-methoxy-1H-indol-3-yl)(oxo)acetate (1.36 g; 5.5 mmol) dissolved in tetrahydrofuran (15 ml) is cooled down to 0° C. Lithium and aluminium hydride in solution in tetrahydrofuran (1M; 16.5 ml; 16.5 mmol) is then added slowly. The reaction mixture is taken to reflux, and stirred for 2 hours. The excess lithium and aluminium hydride is neutralized by the addition of ethyl acetate (1 ml) and distilled water (1 ml). The reaction is filtered whilst warm and the solid is washed with methanol (10 ml). The evaporated filtrate is resolubilized in ethyl acetate (25 ml), washed with an aqueous solution of hydrochloric acid (0.1M; 15 ml) then with a saturated solution of sodium chloride (15 ml). The organic phase is dried over sodium sulphate, followed by evaporating then drying under vacuum. The expected product is obtained in the form of a light yellow oil (0.815 g, yield=78%).

NMR ¹H (DMSO-d₆, 400 MHz) δ: 10.54 (s, 1H, NH); 7.36-7.34 (d, J=8.6 Hz, 1H, arom.); 6.96 (s, 1H, arom.); 6.82 (s, 1H, arom.); 6.63-6.60 (d, J=8.6 Hz, 1H, arom.); 4.57-5.54 (t, J=5.4 Hz, 1H, OH); 3.74 (s, 3H, OCH₃); 3.64-3.59 (m, J=7.4 Hz and J′=5.4 Hz, 2H, CH₂); 2.80-2.76 (t, J=7.4 Hz, 2H, CH₂). MS/LC: m/z=192.17 (M+H) rt=8.27 min (condition 2).

Ethyl indole alcohols of formula R_{3′}(CH₂)₂OH were prepared with the following indole R_{3′}groups:

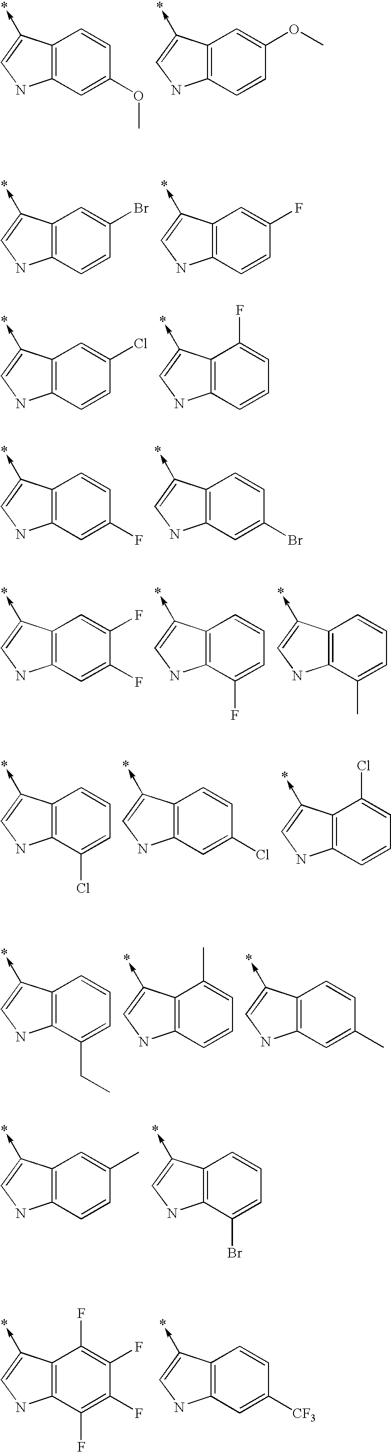

5.2.4 Preparation of the Ethylindole Bromides (8b):

The ethylindole bromides of general formula (8b) can be prepared by bromination of the corresponding alcohols (7b) according to the general methods described above for obtaining benzyl bromides.

### Preparation 14: 3-(2-bromoethyl)-6-methoxy-1H-indole (C₁₁H₁₂BrNO, M=254.13)

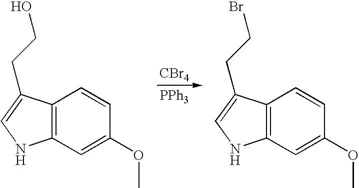

2-(6-methoxy-1H-indol-3-yl)ethanol (0.815 g, 4.3 mmol) and carbon tetrabromide (1.6 g; 5 mmol) dissolved in dichloromethane (25 ml) are cooled down to 0° C. Triphenylphosphine is added (1.3 g; 5 mmol). The reaction mixture is stirred at ambient temperature for 2 hours. The dichloromethane is evaporated off and the residue obtained is purified on silica (eluent: heptane/ethyl acetate: 3/1). The fractions are evaporated and the solid obtained is dried under vacuum (0.69 g; yield=63%).

NMR ¹H (DMSO-d₆, 400 MHz) δ: 10.69 (s, 1H, NH); 7.42-7.39 (d, J=8.6 Hz, 1H, arom.); 7.08 (s, 1H, arom.); 6.84 (s, 1H, arom.); 6.65-6.62 (d, J=8.6 Hz, 1H, arom.); 3.74 (s, 3H, OCH₃); 3.71-3.68 (t, J=7.6 Hz, 2H, CH₂); 3.21-3.17 (t, J=7.6 Hz, 2H, CH₂). MS/LC: m/z=254.04 (M+H) rt=10.56 min (condition 2).

Indole bromides of formula R_{3′}(CH₂)₂Br were prepared with the following indole R₃ groups:

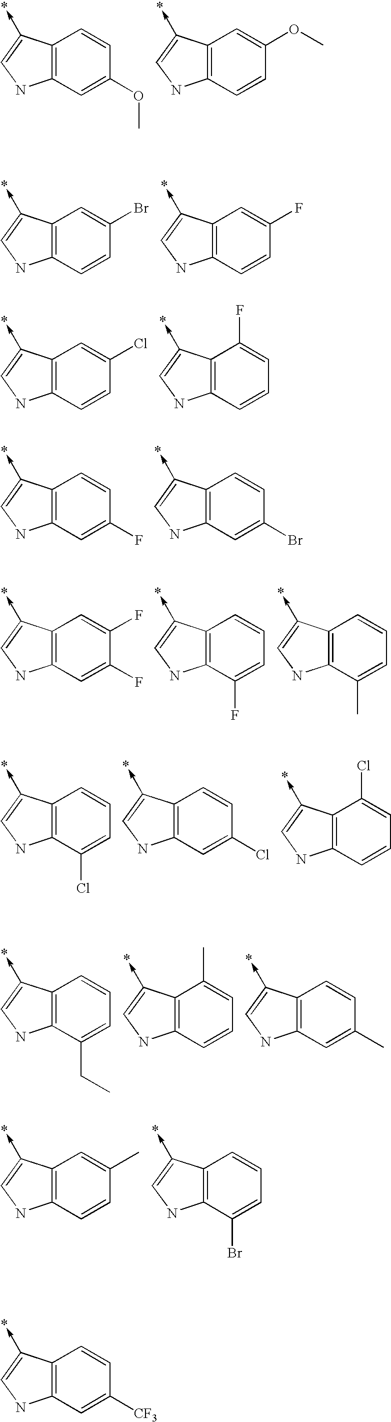

5.3. Preparation of the Brominated Derivatives of General Formula (8d):

The brominated derivatives of general formula Br—(CH₂)_{n′}[Q′]_{p′}[C(X′)(Y′)]_{m′}Z′ where Q′ represents C(O), p′ represents 0 or 1, m′ represents 0, Z′ represents the indolyl group and (CH₂)ₙ has the meaning indicated above, (8d), can be obtained according to methods known to the person skilled in the art, for example by acylation of an indole (O. Ottoni et al. Org. Lett., 2001, 3(7), 1005-1007), followed or not followed by a reduction of the carbonyl group (E. Wenkert et al. J. Org. Chem. 1986, 51(12), 2343-2351).

### Preparation 15: 3-bromo-1-(7-methyl-1H-indol-3-yl)propan-1-one (C₁₂H₁₂BrNO, M=266.14).

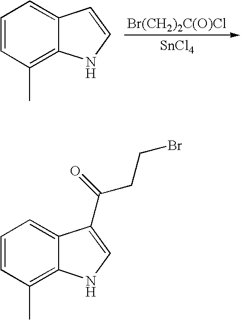

7-methyl-1H-indole (131 mg; 1 mmol) is placed in solution in 2 ml of dichloromethane at 0° C. A molar solution of tin tetrachloride in dichloromethane (1.2 ml; 1.2 mmol) is added at 0° C. then the reaction mixture is stirred at ambient temperature for 30 minutes. 3-bromopropionyl chloride (101 μl; 1 mmol) and nitromethane (1.5 ml) are then added into the medium and the reaction maintained under stirring for 24 hours. Then 5 ml of water is added and the product is extracted with 3 times 5 ml of ethyl acetate. The organic phases are combined, dried over sodium sulphate and the solvents are evaporated off. The solid obtained is dried under reduced pressure (21.5 mg; yield=8%).

NMR ¹H (DMSO-d₆, 400 MHz) δ: 8.39-8.38 (m, 1H, arom.); 8.03-8.01 (d, 1H, arom.); 7.09-7.07 (t, 1H, arom.); 7.02-7.01 (d, 1H, arom.); 3.82-3.79 (t, 2H, CH₂); 3.51-3.48 (t, 2H, CH₂); 2.43 (s, 3H, CH₃). MS/LC: m/z=266.03 rt=9.84 min (condition 1).

The brominated derivatives of general formula (8d) were prepared with the following —(CH₂)_{n′}[Q′]_{p′}[C(X′)(Y′)]m′Z′ groups: 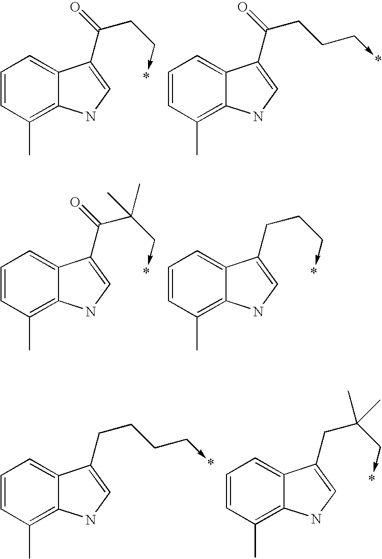
6. Preparation of the Compounds of General Formula (I):

6.1. Substitution of Thiols (6) by Benzyl Bromides (8a):

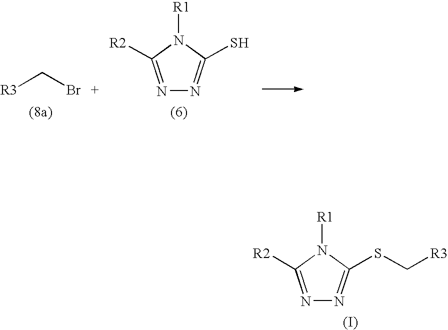

The thiols of general formula (6) can be substituted by benzyl bromides of general formula (8a) after activation of the sulphur atom by a base such as NaOAc, KOH, K₂CO₃ in a protic solvent such as methanol or ethanol (Shetgiri, N. P.; Kokitkar, S. V. Indian J. Chem, Sect B: Org Chem Incl Med Chem 2001, 40 (2), 163-166) or by an organic base such as triethylamine or diisopropylamine in an apolar solvent such as acetone or dichloromethane or also by a base supported on resin such as morpholinomethyl polystyrene resin (Novabiochem) or 7-methyl-1,5,7-triazabicyclo[4,4,0]dec-5-ene polystyrene resin (Novabiochem) after swelling the resin in an aprotic solvent such as dichloromethane. The reaction takes place at ambient temperature over a period ranging from 12 to 36 hours. The excess reagent of general formula (8a) can be trapped by the addition for example of a thiophenol resin (Argonaut) and stirring for 4 to 8 hours. The suspension is filtered, the filtrate is evaporated, followed by purification by chromatography on a silica column. In the case where the amine function present on the molecule is protected by a group of carbamate type (such as, for example, the tert-butoxycarbonyl group), the residue is treated with an acid such as trifluoroacetic acid for 10 to 30 minutes or by a molar solution of hydrochloric acid in ethyl ether for 16 to 20 hours. The final product is then obtained in salified form and in the case of the trifluoroacetate, the salt is treated with a basic resin of Amberlite type then resalified by a molar solution of hydrochloric acid in an aprotic solvent such as ethyl ether, ethyl acetate or dioxane.

### EXAMPLE A

### 2-[3-(benzyl-5-yl)-5-phenyl-4H-1,2,4-triazol-4-yl]ethylamine hydrochloride (C₁₇H₁₉N₄SCl, M=346.88)

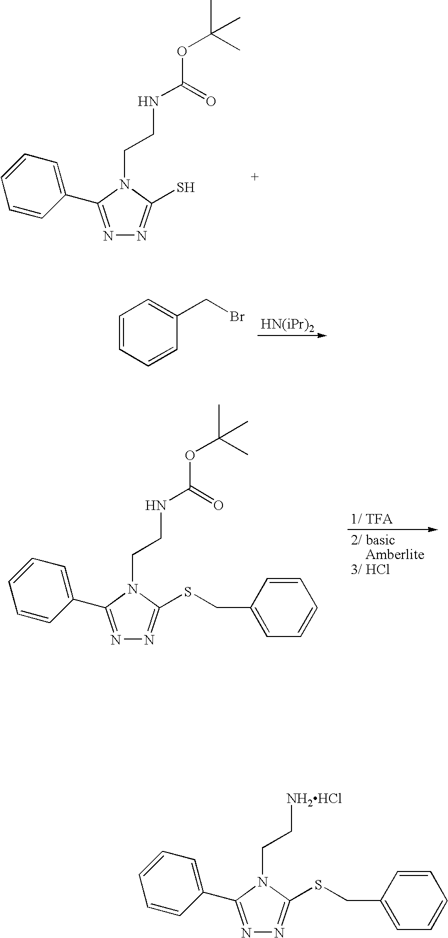

Diisopropylamine (0.14 ml; 1 mmol), then benzyl bromide (0.12 ml; 1 mmol) are added to tert-butyl 2-(3-phenyl-5-sulphanyl-4H-1,2,4-triazol-4-yl)ethylcarbamate (320 mg; 1 mmol) dissolved in tetrahydrofuran (5 ml). The solution is stirred at ambient temperature for 24 hours, then the solvent is evaporated off. Dichloromethane (2 ml) and trifluoroacetic acid (2 ml) are added and the solution obtained is stirred for 10 minutes at ambient temperature. The solvents are evaporated off, the compound redissolved in methanol is passed through a basic Amberlite resin in order to obtain the amine in the form of the free base, followed by purification by chromatography on a silica column (eluent: ethyl acetate/methanol: 1/1). The fractions are evaporated and the hydrochloride of the amine is obtained by treatment with a molar solution of hydrochloric acid in ethyl ether (1 ml; 1 mmol). The precipitate formed is filtered, followed by washing with ethyl ether then drying under vacuum (80 mg; yield=23%).

NMR ¹H (DMSO-d₆, 400 MHz) δ: 8.22 (broad s, 3H, NH₃⁺); 7.66-7.63 (m, 2H, arom.); 7.58-7.56 (m, 3H, arom.) 7.41-7.40 (m, 2H, arom.); 7.35-7.29 (m, 3H, arom.); 4.46 (s, 2H, CH₂); 4.16 (t, J=8.3 Hz, 2H, CH₂); 2.88-2.82 (m, J=2 and 8.3 Hz, 2H, CH₂). MS/LC: m/z=311.13 (M+H) rt=6.51 min (condition 1).

Benzyl bromides (8a) of general formula R₃Br were used with the following R₃ groups:

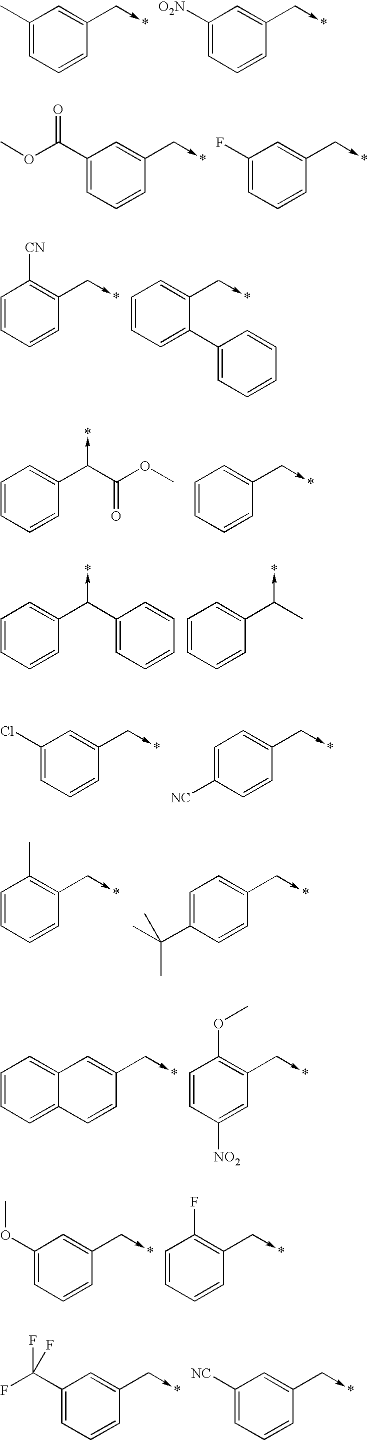

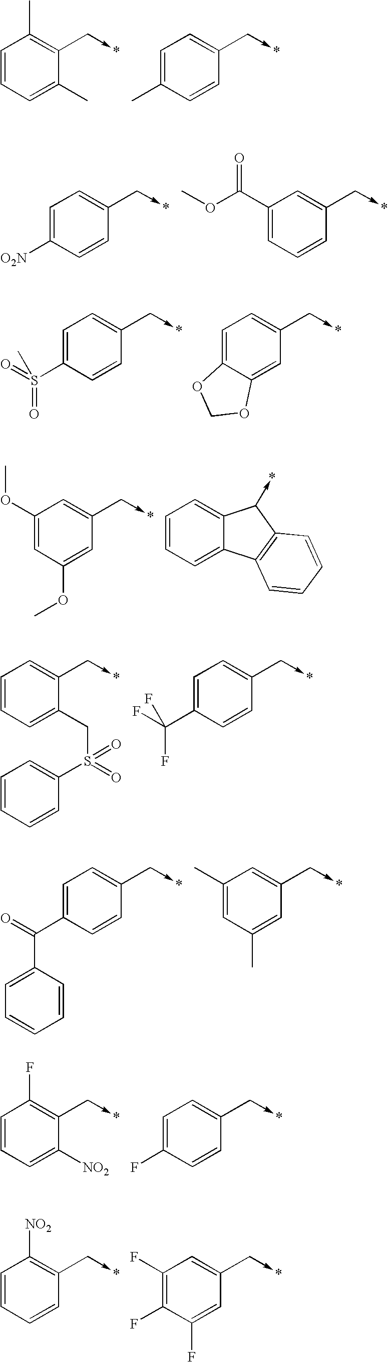

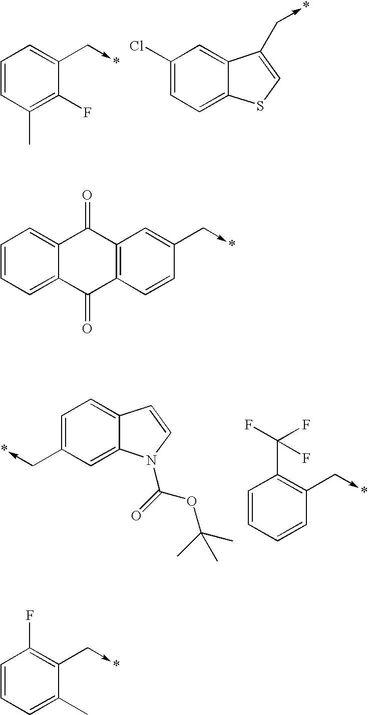

6.2. Substitution of the Thiols (6) by α-bromoketones (8c):

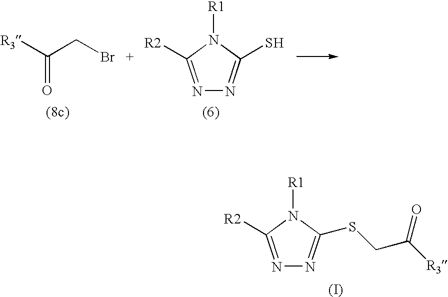

The thiols of general formula (6) can be substituted by α-bromoketones of general formula (8c) after activation of the sulphur atom under the same conditions as those described previously. The reaction takes place at ambient temperature over a period ranging from 12 to 24 hours. The excess reagent of general formula (8c) can be trapped by the addition for example of a thiophenol resin (Argonaut) or a resin of aminomethyl-polystyrene type (Novabiochem) and stirring is carried out for 4 to 8 hours. The suspension is filtered, the filtrate evaporated and purified on a silica column. In the case where the amine function present on the molecule is protected by a group of carbamate type (such as, for example, the tert-butoxycarbonyl group), the residue is treated with a molar solution of hydrochloric acid in ethyl ether for 16 to 20 hours. The final product is then obtained, after purification on a silica column if necessary, in the form of the hydrochloride.

### EXAMPLE B

### 2-{[4-(6-aminohexyl)-5-(2-naphthyl)-4H-1,2,4-triazol-3-yl]sulphanyl}-1-[4-(diethylamino)phenyl]ethanone hydrochloride (C₃₀H₃₈N₅OSCl, M=552.19)

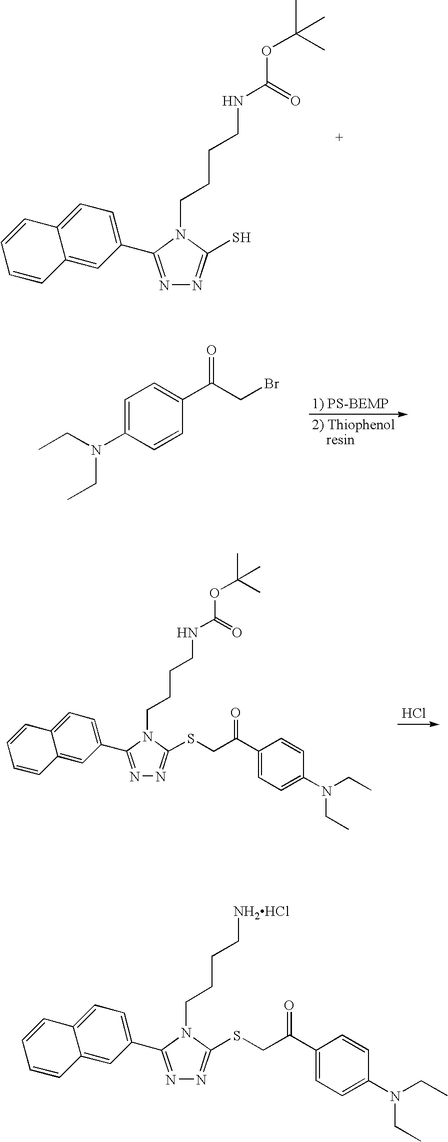

2-tert-butylimino-2-diethylamino-1,3-dimethyl-perhydro-1,3,2-diazophosphorine on polystyrene resin is added to tert-butyl 6-[3-(2-naphthyl)-5-sulphanyl-4H-1,2,4-triazol-4-yl]hexylcarbamate (30 mg; 0.07 mmol) dissolved in tetrahydrofuran (1 ml). The suspension is stirred at ambient temperature for 30 minutes then 2-bromo-1-[4-(diethylamino)phenyl]ethanone (22 mg; 0.08 mmol) is added to the medium. The mixture is stirred at ambient temperature for 16 hours. The excess 2-bromo-1-[4-(diethylamino)phenyl]ethanone is trapped by the addition of a thiophenol resin (70 mg, 0.1 mmol, Argonaut) and stirring for 6 hours. The suspension is filtered and the filtrate evaporated. To deprotect the amine function, the filtrate is solubilized in methanol (0.5 mmol) then a molar solution of hydrochloric acid in ethyl ether is added (2 ml; 2 mmol). The solution is stirred for 16 hours, then evaporated. The resulting solid is dried under vacuum (28 mg; yield=63%). MS/LC: m/z=516.40 (M+H) rt=8.60 min (condition 1).

Bromoketones (8c) of general formula R₃Br were used with the following R₃ groups:

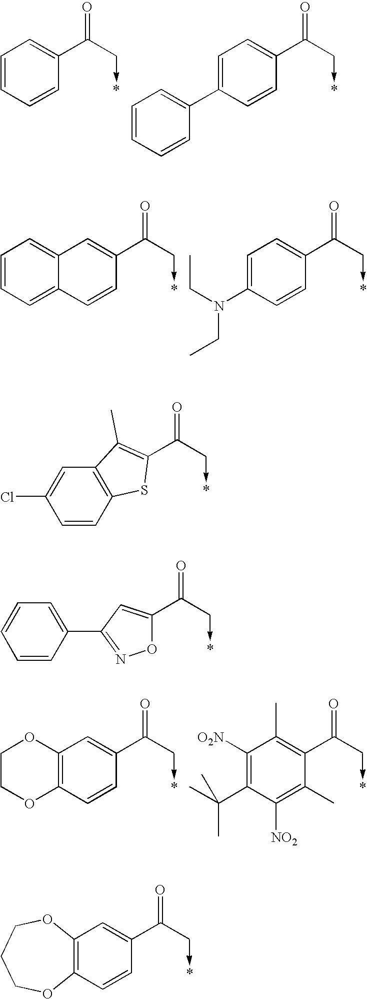

6.3. Substitution of Thiols (6) by Aliphatic Halides Ethylindole Bromides (8b) or Brominated Derivatives of General Formula (8d):

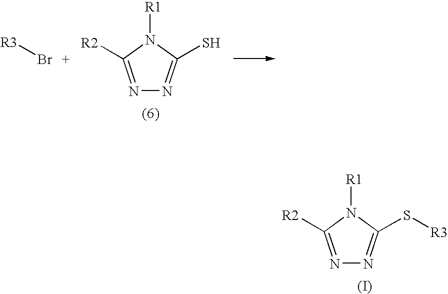

The thiols of general formula (6) can be substituted by aliphatic halides or ethylindole bromides of general formula (8b) or brominated derivatives of general formula (8d) after activation of the sulphur atom by 2-tert-butylimino-2-diethylamino-1,3-dimethyl-perhydro-1,3,2-diaza-phosphorine on polystyrene resin (Fluka). The reaction takes place at ambient temperature over a period ranging from 3 to 6 hours. The suspension is filtered, the filtrate evaporated and purified on a silica column. In the case where the amine function present on the molecule is protected by a group of carbamate type (such as, for example, the tert-butoxycarbonyl group), the residue is treated with a molar solution of hydrochloric acid in ethyl ether for 16 to 20 hours. The final product is then obtained in the form of the hydrochloride.

### EXAMPLE C

### 3 [3-{[2-(1H-indol-3-yl)ethyl]sulphanyl}-5-(2-naphthyl)-4H-1,2,4-triazol-4-yl]propylamine hydrochloride (C₂₆H₂₈N₅SCl, M=478.06)

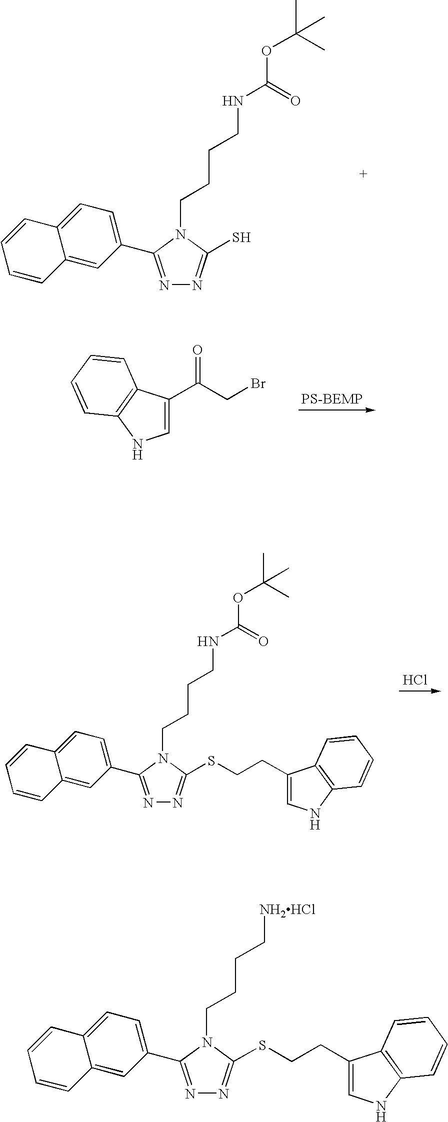

### Stage 1: tert-butyl-3 [3-{[2-(1H-indol-3-yl)ethyl]sulphanyl}-5-(2-naphthyl)-4H-1,2,4-triazol-4-yl]propylcarbamate (C₃₁H₃₅NSO₂S, M=541.72)

2-tert-butylimino-2-diethylamino-1,3-dimethyl-perhydro-1,3,2-diazaphosphorine on polystyrene resin (0.91 g, 2 mmol, 2.2 mmol/g, Fluka) is added to 265 mg (0.66 mmol) of tert-butyl 4-[3-(2-naphthyl)-5-sulphanyl-4H-1,2,4-triazol-4-yl]butylcarbamate in anhydrous tetrahydrofuran (15 ml). The suspension is stirred for 10 minutes at ambient temperature, then 3-(2-bromomethyl)indole (149 mg, 0,66 mmol) is added. The reaction mixture obtained is stirred at ambient temperature for 4 hours, then filtered. The evaporated filtrate is purified by flash chromatography on a silica column (ethyl acetate/heptane 2:1). The fractions are recombined, followed by evaporation and the white residue is dried under vacuum (249 mg, yield=70%).

NMR ¹H (DMSO-d₆, 400 MHz) δ: 8.41 (broad s, 1H, NH); 8.01 (s, 1H, arom.); 7.99-7.96 (m, 1H, arom.); 7.92-7.91 (m, 2H, arom.); 7.69-7.65 (m, 2H, arom.); 7.60-7.57 (m, 2H, arom.); 7.39-7.37 (m, 1H, arom.); 7.22-7.20 (m, 1H, arom.); 7,14-7.11 (m, 2H, arom.); 4.44 (broad s, 1H, NH); 3.91 (t, J=8 Hz, 2H, CH₂); 3.70 (t, J=5.9 Hz, 2H, CH₂); 3.33 (t, J=5.9 Hz, 2H, CH₂); 2.99-2.97 (m, 2H, CH₂); 1.61-1.57 (m, 2H, CH₂); 1.42 (s, 9H, (CH₃)₃); 1.36-1.27 (m, 2H, CH₂) MS/LC: m/z=542.36 (M+H) rt=11.07 min (condition 1).

### Stage 2: 3[3-{[2-(1H-indol-3-yl)ethyl]sulphanyl}-5-(2-naphthyl)-4H-1,2,4-triazol-4-yl]propylamine hydrochloride (C₂₆H₂₈N₅SCl, M=478.06)

The tert-butyl-3 [3-{[2-(1H-indol-3-yl)ethyl]sulphanyl}-5-(2-naphthyl)-4H-1,2,4-triazol-4-yl]propylcarbamate formed previously is dissolved in anhydrous dichloromethane (3 ml) and methanol (2 ml) then a molar solution of hydrochloric acid in ethyl ether (3.1 ml) is added to the solution. The mixture is stirred for 45 minutes then evaporated and the beige solid obtained is dried under vacuum (188 mg, yield=94%).

NMR ¹H (D₂O, 770 C, 400 MHz) δ: 8.59-8.56 (m, 1H, arom.); 8.51-8.49 (m, 2H, arom.); 8.41 (s, 1H, arom.); 8.19-8.16 (m, 2H, arom.); 8.04-8.02 (m, 1H, arom.); 7.96-7.93 (m, 1H, arom.); 7.87-7.84 (m, 1H, arom.); 7.68 (s, 1H, arom.); 7.65-7.63 (m, 1H, arom.); 7.58-7.56 (m, 1H, arom.); 4.28 (t, J=8.3 Hz, 2H, CH₂); 4.17 (t, J=5.5 Hz, 2H, CH₂); 3.72 (t, J=5.5 Hz, 2H, CH₂); 3.14 (t, J=8.3 Hz, 2H, CH₂); 1.92-1.85 (m, J=8.3 Hz and 7 Hz, 2H, CH₂); 1.82-1.74 (m, J=8.3 Hz and 7 Hz, 2H, CH₂). MS/LC: m/z=442.26 (M+H), rt=8.14 min (condition 1).

Aliphatic halides, brominated derivatives (8b) or (8d) of general formula R₃Br were used with the following R₃ groups:

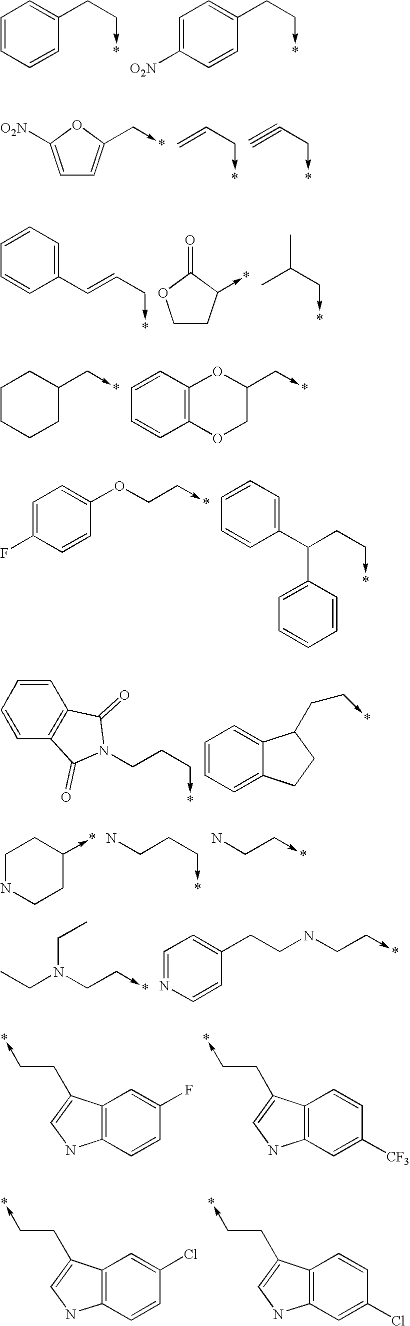

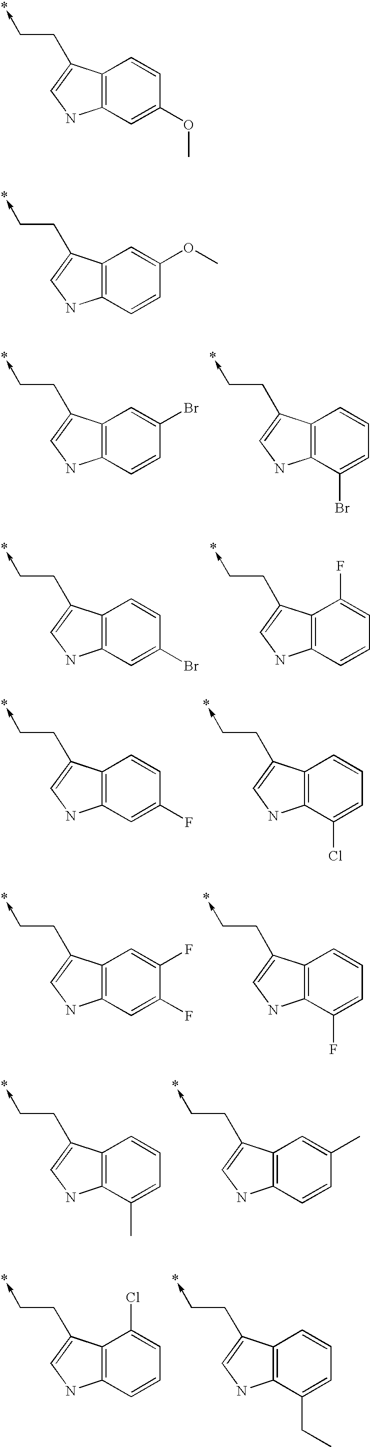

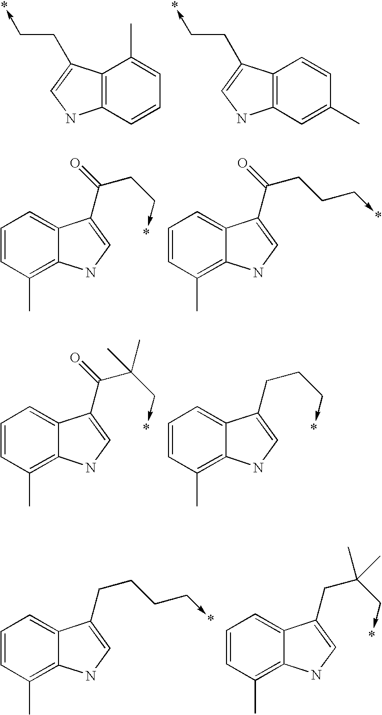

6.4. The Particular Case Where R₃ Includes an Amide Function:

The compounds of general formula (I) such that R₃ is a radical of formula —CH₂—C(O)—NH—(CH₂)ₘ—NXY, where m, X and Y are as defined above, can be obtained in 3 stages starting from the thiol of general formula (6).

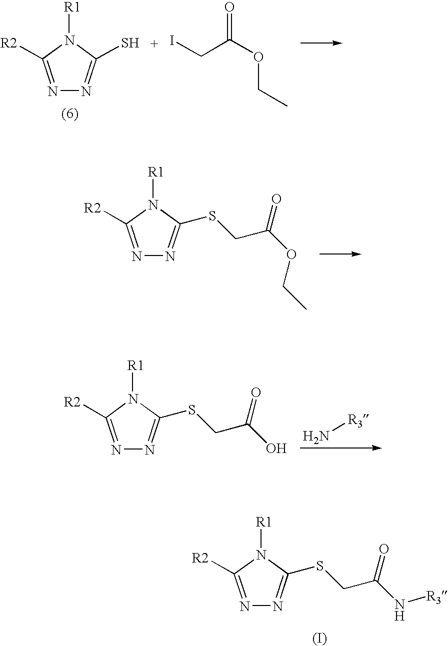

6.4.1. Substitution of Sulphur and Hydrolysis of the Ester:

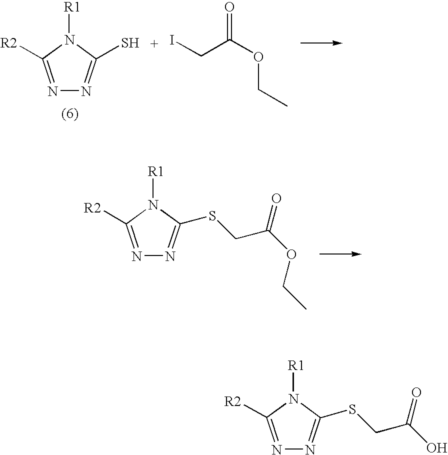

The thiols of general formula (6) can be substituted by ethyl iodoacetate after activation of the sulphur atom by a base such as NaH or by use of 2-tert-butylimino-2-diethylamino-1,3-dimethyl-perhydro-1,3,2-diazaphosphorine on polystyrene resin (Fluka) in an aprotic solvent such as dichloromethane or dimethylformamide. The reaction takes place at ambient temperature over a period ranging from 12 to 24 hours, then the reaction mixture is washed, followed by concentration under vacuum. The ester is then hydrolyzed by treatment with a base such as, for example an aqueous solution of KOH or lithium hydroxide in the presence of an aprotic solvent such as tetrahydrofuran at ambient temperature over a period ranging from 3 to 6 hours (Baldwin, J. E.; Adlington, R. M.; Ramcharitar, S. H. J Chem Soc, Chem Commun 1991 (14), 940-942). The corresponding acid is obtained after evaporation of the solvents, neutralization with an aqueous solution of hydrochloric acid, extraction with an organic solvent such as ethyl acetate and used in the following stages without other purification.

### Preparation 16: {[4-(2,2-diphenylethyl)-5-(2-naphthylmethyl)-4H-1,2,4-triazol-3-yl]sulphanyl}acetic Acid (C₂₉H₂₅N₃O₂S, M=479.61)

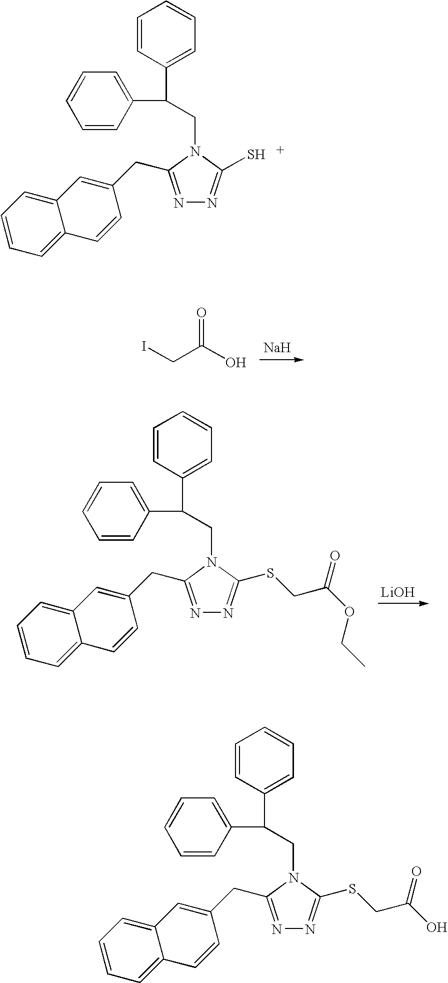

Sodium hydride (0.4 g; 10 mmol) is added to 4-(2,2-diphenylethyl)-5-(2-naphthylmethyl)-4H-1,2,4-triazol-3-thiol (4 g; 9.5 mmol) dissolved in dichloromethane (100 ml); the solution is stirred at ambient temperature for 30 minutes. Ethyl iodoacetate is added (1.2 ml; 10 mmol) and the mixture is stirred at ambient temperature for 16 hours. The reaction mixture is washed with distilled water (50 ml) then with a saturated solution of sodium chloride (50 ml). The organic phase is evaporated. The acid is obtained by hydrolysis: lithium hydroxide (1.1 g; 27 mmol) dissolved in distilled water (40 ml) is added to the residue dissolved in tetrahydrofuran (80 ml), and this mixture is stirred at ambient temperature for 4 hours. The solvents are evaporated off then a normal solution of hydrochloric acid is added until the pH is slightly acid. This solution is extracted twice with ethyl acetate (50 ml), the organic phases are combined, dried over sodium sulphate followed by filtration, evaporation and the solid obtained is dried under vacuum (2 g, yield=44%) before being used in the following stage.

NMR ¹H (DMSO-d₆, 400 MHz) δ: 12.92 (broad s, 1H, C(O)—OH); 7.92-7.90 (m, 1H, arom.); 7.83-7.81 (m, 1H, arom.); 7.55-7.37 (m, 4H, arom.); 7.34-7.23 (m, 10H, arom.); 7.11-7.09 (m, 1H, arom.); 4.64 (d, J=9 Hz, 2H, CH₂); 4.38 (t, J=9 Hz, 1H, CH); 3.96 (s, 2H, CH₂); 3.92 (s, 2H, CH₂). MS/LC: m/z=480.28 (M+H), rt=10.75 min (condition 1).

6.4.2. Peptide Coupling:

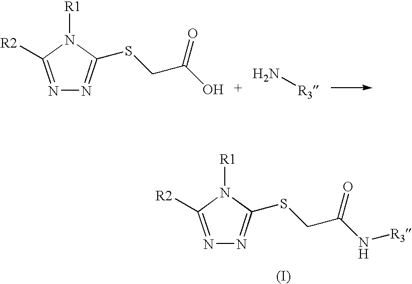

The compounds of general formula (I) such that R₃ is a radical of formula —CH₂—C(O)—NH—(CH₂)ₘ—NXY, where m, X and Y are as defined above, can be obtained by standard methods of peptide synthesis (M. Bodansky, The Practice of Peptide Synthesis, 145 (Springer-Verlag, 1984)), for example in tetrahydrofuran, dichloromethane or dimethylformamide in the presence of a coupling reagent such as cyclohexylcarbodiimide (DCC), 1,1′-carbonyldiimidazole (CDI) (J. Med. Chem. 1992, 35 (23), 4464-4472), 1-(3-dimethylaminopropyl)-3-ethylcarbodiimide hydrochloride (EDC or WSCI) (John Jones, the chemical synthesis of peptides, 54 (Clarendon Press, Oxford, 1991)) or benzotriazol-1-yl-oxy-tris-pyrrolidino-phosphonium hexafluorophosphate (PyBOP) (Coste, J.; The-Nguyen, D.; Castro, B.; Tetrahedron Lett 1990, 31, 205). The compound of general formula (I) is obtained after purification on a silica column.

### EXAMPLE D

### 2-{[4-(2,2-diphenylethyl)-5-(2-naphthylmethyl)-4H-1,2,4-triazol-3-yl]sulphanyl}-N-[3-(4-methyl-1-piperazinyl)propyl]acetamide (C₃₇H₄₂N₆OS, M=618.85)

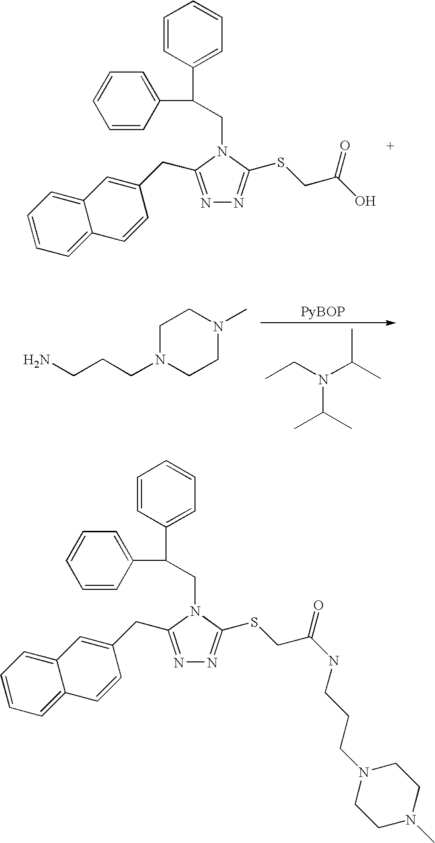

Benzotriazol-1-yl-oxy-tris-pyrrolidino-phosphonium hexafluorophosphate (52 mg; 0.1 mmol) is added to {[4-(2,2-diphenylethyl)-5-(2-naphthylmethyl)-4H-1,2,4-triazol-3-yl]sulphanyl}acetic acid (48 mg; 0.1 mmol) dissolved in dichloromethane (5 ml). The solution is stirred at ambient temperature for 30 minutes then diisopropyl-ethyl-amine (38 μl; 0.22 mmol) and 3-(4-methyl-1-piperazinyl)propylamine (20 μl; 0.12 mmol) are added. The mixture is stirred under argon at ambient temperature for 16 hours. The solvents are evaporated off and the residue is purified by chromatography on a silica column (eluent: dichloromethane/methanol 95/5). After evaporation of the fractions, the solid obtained is dried under vacuum (7 mg, yield=11%). MS/LC: m/z=619.41 (M+H), rt=8.37 min (condition 1).

The following groups of R₃′″ type were used:

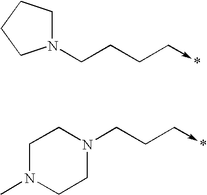

A subject of the invention is also a process for the preparation, in liquid phase, of the compounds of formula I according to the invention, characterized in that it includes the reaction of isothiocyanates of formula R₁-NCS on hydrazides of formula R₂—C(O)—NH—NH₂ in which R₁ and R₂ have the meaning indicated above, in order to obtain the compounds of formula (5) 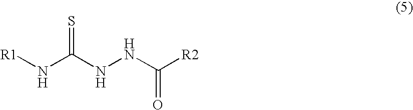
which compounds of formula (5) can be subjected to a basic treatment in order to obtain the corresponding compounds of formula (6) 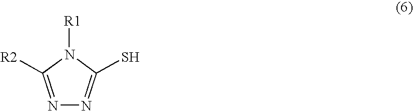
which compounds of formula (6) are reacted with
A) either a compound of formula Br—(CH₂)_{n′}[Q′]_{p′}[C(X′)(Y′)]_{m′}Z′ where n′=1, p′=m′=0 and Z′ has the meaning indicated above in order to obtain, after deprotection of the amine function present on the molecule, the corresponding compound of formula (I),
B) or a compound of formula Br—(CH₂)_{n′}[Q′]_{p′}[C(X′)(Y′)]m′Z′ where n′=1, Q′=—C(O)—, m′=0 and Z′ has the meaning indicated above in order to obtain, after deprotection of the amine function present on the molecule, the corresponding compound of formula (I),
C) or a compound of formula Br—(CH₂)_{n′}[Q′]p′[C(X′)(Y′)]_{m′}Z′ where Q′, X′, Y′, Z′, n′, p′ and m′ have the meaning indicated above in order to obtain, after deprotection of the amine function present on the molecule, the corresponding compound of formula (I).

Compounds I of the present invention have useful pharmacological properties. Thus it has been discovered that compounds I of the present invention have a high affinity for one (or more) of the somatostatin receptors. They can be used as non-peptide agonists or antagonists of somatostatin in a selective or non-selective manner.

The compounds of the present invention can therefore be used in different therapeutic applications. They can advantageously be used to treat the pathological conditions or the diseases such as presented above and in which one (or more) of the somatostatin receptors is (are) involved.

An illustration of the pharmacological properties of the compounds of the invention will be found hereafter in the experimental part.

The compounds of the invention are also analogues of urotensin II and are thus particularly useful for treating pathological conditions or diseases in which urotensin II is involved.

A subject of the present Application is also pharmaceutical compositions containing, as active ingredient, at least one of the products of formula 1 as defined above as well as the addition salts with pharmaceutically acceptable mineral or organic acids of said products of formula I, combined with a pharmaceutically acceptable support.

The compounds of formula I in which either R₁ represents (CH₂)₂—W and W representing morpholino or piperazinyl, R₂ phenyl, m-chlorophenyl or 4-pyridyl, and R₃ the hydrogen atom, or R₁ represents (CH₂)₂—W and W representing pyrrolidinyl, R₂ p-chlorophenyl and R₃ the hydrogen atom, have been described in Phosphorus, Sulfur and Silicon, 2000, vol. 164 pp, 67-81, but only as synthesis intermediates and no therapeutic activity has been envisaged for these compounds.

A subject of the present invention is therefore also a pharmaceutical composition containing, as active ingredient, combined with a pharmaceutically acceptable support, at least one compound of general formula 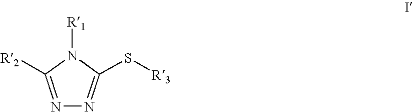
in racemic, enantiomeric form or all combinations of these forms, in which
one of the R′₁, R′₂ or R′₃ radicals represents a radical of formula —(CH₂)ₙ—[Q]ₚ—(CH₂)ₘ—NXY or —(CH₂)ₙ—W in which
   W represents a heterocycloalkyl containing at least one nitrogen atom;
   Q represents —O—, —S—, —C(O)—NH—, —C(Z_{q})(Z_{q}′), aryl or (C₃-C₇)cycloalkyl;
   Z_{q} and Z_{q}′ represent, independently, the hydrogen atom, aryl optionally substituted by aryl, (C₃-C₇)cycloalkyl-alkyl, arylalkyl, —C(O)O—R or —C(O)—NH—R′;
   R represents a (C₁-C₆)alkyl, aryl or aralkyl radical, aryl and aralkyl being optionally substituted by one or more identical or different substituents chosen from: (C₁-C₆)alkoxy, hydroxy, halo, nitro cyano, amino, (C₁-C₆)alkylamino and di((C₁-C₆)alkyl)amino;
   R′ represents a (C₁-C₆)alkyl, aryl, aralkyl, heteroaryl or heteroaryl-alkyl radical, the aryl, aralkyl, heteroaryl and heteroaryl-alkyl radicals being optionally substituted by one or more identical or different substituents chosen from: (C₁-C₆)alkoxy, hydroxy, halo, nitro cyano, amino, (C₁-C₆)alkylamino, di((C₁-C₆)alkyl)amino;
   X and Y represent, independently, the hydrogen atom, (C₁-C₆)alkyl, (C₁-C₆)alkoxy-carbonyl or a heteroaryl-alkyl, or X and Y form together with the nitrogen atom on which they are attached, a heterocycloalkyl optionally substituted by a (C₁-C₆)alkyl;
   p represents 0 or 1; n and m represent, independently, an integer from 0 to 6;
and the two other radicals represent, independently, a radical of formula —(CH₂)ₙ [Q′]_{p′}[C(X′)(Y′)]_{m′}Z′ in which
   Q′ represents —O—, —S—, —C(O)—, —NH—, —CH═CH— or —C≡C—
   X′, Y′ and Z′ represent, independently, a hydrogen atom, (C₁-C₆)alkyl, (C₁-C₆)alkoxy, (C₁-C₆)alkoxy-carbonyl, cyano, amino, (C₁-C₆)alkylamino, di((C₁-C₆)alkyl)amino, (C₃-C₇)cycloalkyl, heterocycloalkyl, aryl or heteroaryl, or a radical of formula 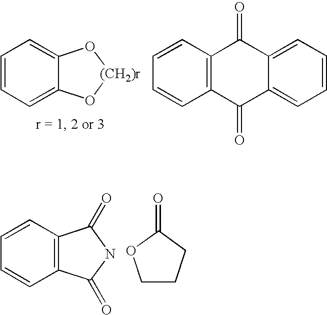
   the (C₃-C₇)cycloalkyl, heterocycloalkyl, aryl and heteroaryl radicals being optionally substituted by one or more identical or different substituents chosen from: —(CH₂)_{q}—X″—Y″, hydroxy, halo, nitro, cyano, amino, (C₁-C₆)alkylamino and di((C₁-C₆)alkyl)amino;
   X″ represents —O—, —S—, —C(O)—, —C(O)—O—, —SO₂— or a covalent bond;
   Y″ represents a (C₁-C₆)alkyl radical optionally substituted by one or more identical or different halo radicals; or aryl or heteroaryl optionally substituted by one or more identical or different substituents chosen from: (C₁-C₆)alkoxy, hydroxy, halo, nitro cyano, amino, (C₁-C₆)alkylamino and di((C₁-C₆)alkyl)amino;
   p′ represents 0 or 1, and n′, m′ and q′ represent, independently, an integer from 0 to 6;

A pharmaceutical composition according to the invention can be in the form of a solid, for example, powders, granules, tablets, gelatin capsules or suppositories. Appropriate solid supports can be, for example, calcium phosphate, magnesium stearate, talc, sugars, lactose, dextrin, starch, gelatin, cellulose, methylcellulose, the sodium carboxymethyl cellulose, polyvinylpyrrolidine and wax.

The pharmaceutical compositions containing a compound of the invention can also be present in liquid form, for example, solutions, emulsions, suspensions or syrups. Appropriate liquid supports can be, for example, water, organic solvents such as glycerol or the glycols, as well as their mixtures, in varying proportions, in water, with added pharmaceutically acceptable oils or fats. The sterile liquid compositions can be used for intramuscular, intraperitoneal or sub-cutaneous injections and the sterile compositions can also be administered intravenously.

All the technical and scientific terms used in the present text have the meaning known to the person skilled in the art. Moreover, all the patents (or patent applications) as well as the other bibliographical references are incorporated by way of reference.

Experimental Part:

Other compounds according to the invention obtained according to the procedures of Examples A, B, C and D described previously, are given in the table below.

The compounds are characterized by their retention time (rt), expressed in minutes, determined by liquid chromatography (LC) and their molecular peak (M+H)⁺ determined by mass spectrometry (MS). For the mass spectrometry, a single quadrupole mass spectrometer (Micromass, Platform model) provided with an electrospray source is used with a resolution of 0.8 Da at 50% valley.

The conditions for the examples presented, are the following:

Eluent: A: Water+0.02% trifluoroacetic acid; B: Acetonitrile

Condition 1 (C1):

| T (min) | A (%) | B (%) |
|---|---|---|
| 0 | 95 | 5 |
| 8.5 | 10 | 90 |
| 10.5 | 10 | 90 |
| 10.6 | 95 | 5 |
| 15 | 95 | 5 |

Flow rate: 1.0 ml/min
Injection: 10 μl
Ambient temperature
Wavelength (% UV): 220 nm
Column: Uptisphere HDO 3 μm 75 * 4.6 mm i.d.

Condition 2 (C2):

| T (min) | A (%) | B (%) |
|---|---|---|
| 0 | 100 | 0 |
| 6 | 20 | 80 |
| 8 | 20 | 80 |
| 8.1 | 100 | 0 |
| 10 | 100 | 0 |

Flow rate: 1.0 ml/min
Injection: 5 μl
Ambient temperature
Wavelength (% UV): 220 nm
Column: Uptisphere ODS 3 μm 50 * 4.6 mm i.d

The conditions according to the examples, are the following:

| Examples | Conditions | Examples | Conditions | Examples | Conditions |
|---|---|---|---|---|---|
| 1 to 15 | 1 | 163 to 164 | 1 | 374 to 466 | 1 |
| 16 to 30 | 2 | 165 to 191 | 2 | 467 to 489 | 2 |
| 31 to 45 | 1 | 192 to 210 | 1 | 490 | 1 |
| 46 to 59 | 2 | 211 to 213 | 2 | 491 to 495 | 2 |
| 60 | 1 | 214 | 1 | 496 to 533 | 1 |
| 61 to 81 | 2 | 215 to 234 | 2 | 534 to 537 | 2 |
| 82 to 98 | 1 | 235 to 236 | 1 | 538 to 551 | 1 |
| 99 to 145 | 2 | 237 to 260 | 2 | 552 | 2 |
| 146 to 151 | 1 | 261 | 1 | 553 to 582 | 1 |
| 152 to 153 | 2 | 262 to 269 | 2 | 583 to 638 | 1 |
| 155 | 1 | 270 to 368 | 1 | 639 to 708 | 1 |
| 155 to 162 | 2 | 369 to 373 | 2 | | |

These examples are presented in order to illustrate the above procedures and must in no case be considered as a limit to the scope of the invention.

In each illustration of the R₁, R₂ and R₃ radicals, the X₁, X₂ and X₃ radicals represent, respectively, the residual part of the compound of formula (I).

| | R1 | R2 | R3 | RT | MH+ |
|---|---|---|---|---|---|
| 1 | 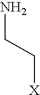 | 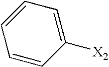 | 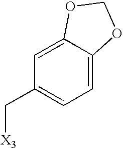 | 7.46 | 355.23 |
| 2 | 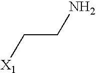 | 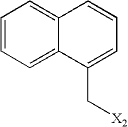 | 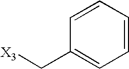 | 7.39 | 375.27 |
| 3 | 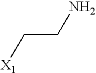 | 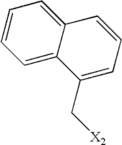 | 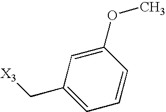 | 7.11 | 405.33 |
| 4 | 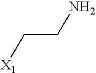 | 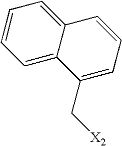 | 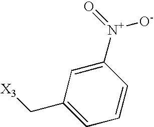 | 7.17 | 420.33 |
| 5 | 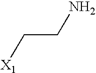 | 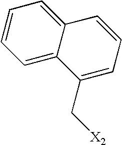 | 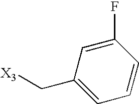 | 7.29 | 393.21 |
| 6 | 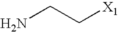 | 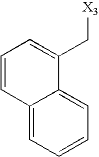 | 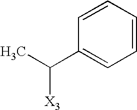 | 7.26 | 389.36 |
| 7 | 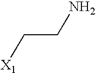 | 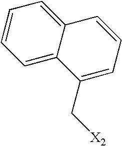 | 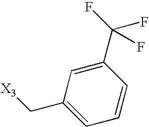 | 7.70 | 443.21 |
| 8 | 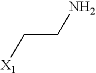 | 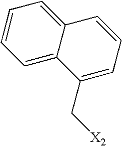 | 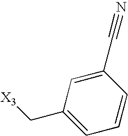 | 7.02 | 400.33 |
| 9 | 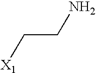 | 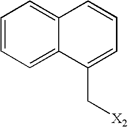 | 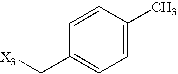 | 7.30 | 389.29 |
| 10 | 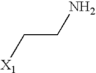 | 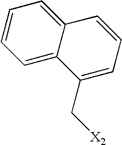 | 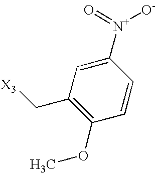 | 7.22 | 450.35 |
| 11 | 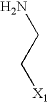 | 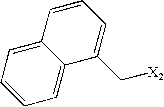 | 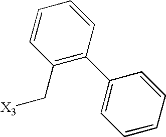 | 7.72 | 451.36 |
| 12 | 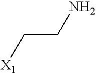 | 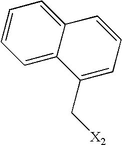 | 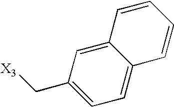 | 7.50 | 425.35 |
| 13 | 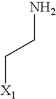 | 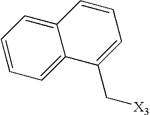 | 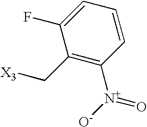 | 7.14 | 438.32 |
| 14 | 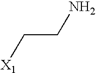 | 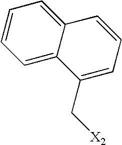 | 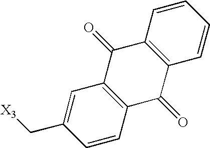 | 7.56 | 505.32 |
| 15 | 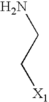 | 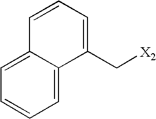 | 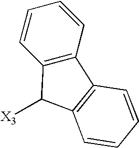 | 7.75 | 449.37 |
| 16 | 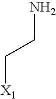 | 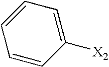 | 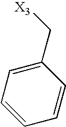 | 3.71 | 311.24 |
| 17 | 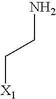 | 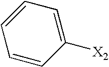 | 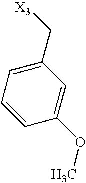 | 3.84 | 341.21 |
| 18 | 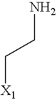 | 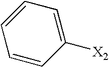 | 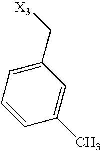 | 3.97 | 325.24 |
| 19 | 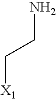 | 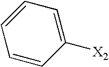 | 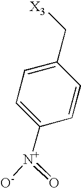 | 3.86 | 356.20 |
| 20 | 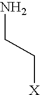 | 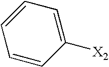 | 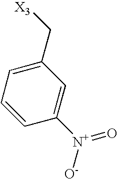 | 3.82 | 356.20 |
| 21 | 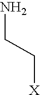 | 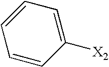 | 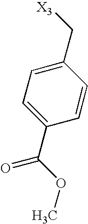 | 3.82 | 369.21 |
| 22 | 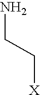 | 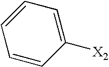 | 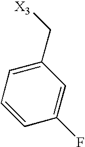 | 3.82 | 329.20 |
| 23 | 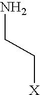 | 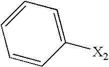 | 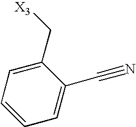 | 3.62 | 336.22 |
| 24 | 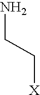 | 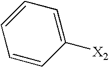 | 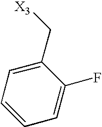 | 3.79 | 329.17 |
| 25 | 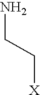 | 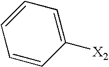 | 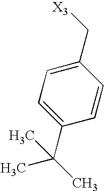 | 4.60 | 367.20 |
| 26 | 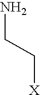 | 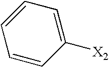 | 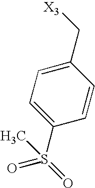 | 3.37 | 389.16 |
| 27 | 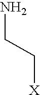 | 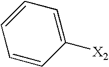 | 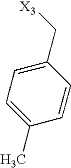 | 3.99 | 325.25 |
| 28 | 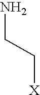 | 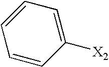 | 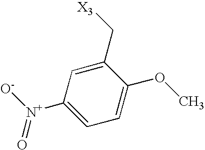 | 3.90 | 386.10 |
| 29 | 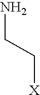 | 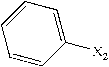 | 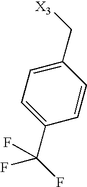 | 4.36 | 379.18 |
| 30 | 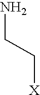 | 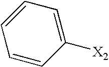 | 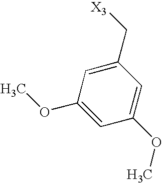 | 3.90 | 371.23 |
| 31 | 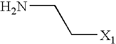 | 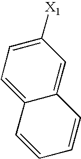 | 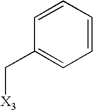 | 7.31 | 361.28 |
| 32 | 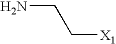 | 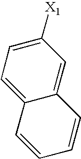 | 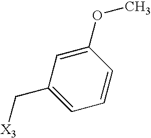 | 7.03 | 391.31 |
| 33 | 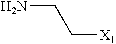 | 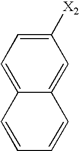 | 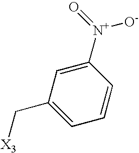 | 7.09 | 406.32 |
| 34 | 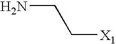 | 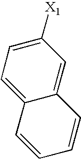 | 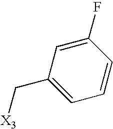 | 7.21 | 379.21 |
| 35 | 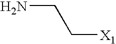 | 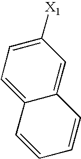 | 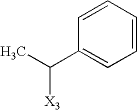 | 7.11 | 375.38 |
| 36 | 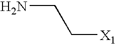 | 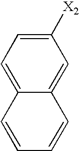 | 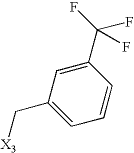 | 7.61 | 429.18 |
| 37 | 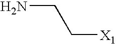 | 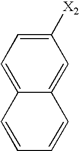 | 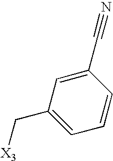 | 6.92 | 386.32 |
| 38 | 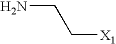 | 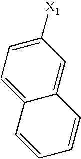 | 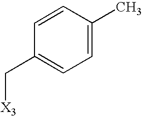 | 7.27 | 375.30 |
| 39 | 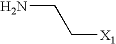 | 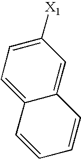 | 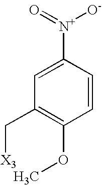 | 7.13 | 436.32 |
| 40 | 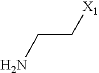 | 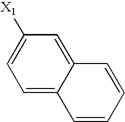 | 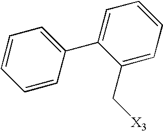 | 7.64 | 437.33 |
| 41 | 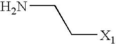 | 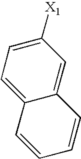 | 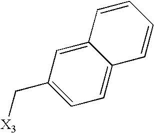 | 7.41 | 411.33 |
| 42 | 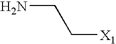 | 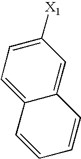 | 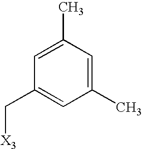 | 7.45 | 389.37 |
| 43 | 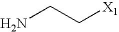 | 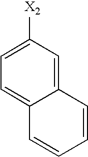 | 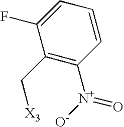 | 7.04 | 424.30 |
| 44 | 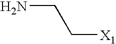 | 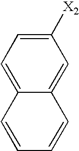 | 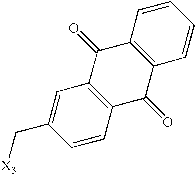 | 7.48 | 491.29 |
| 45 | 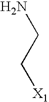 | 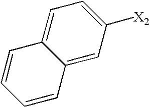 | 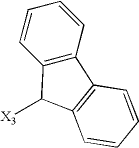 | 7.73 | 435.35 |
| 46 | 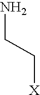 | 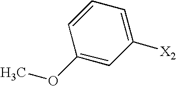 | 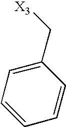 | 3.87 | 341.25 |
| 47 | 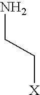 | 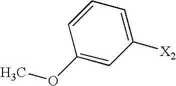 | 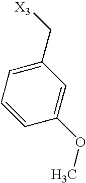 | 3.95 | 371.28 |
| 48 | 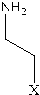 | 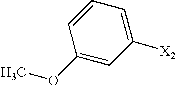 | 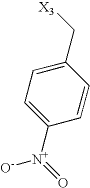 | 402 | 386.18 |
| 49 | 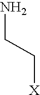 | 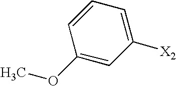 | 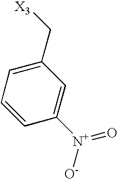 | 3.98 | 386.18 |
| 50 | 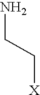 | 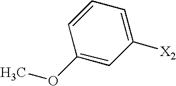 | 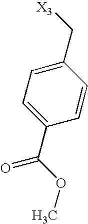 | 3.96 | 399.20 |
| 51 | 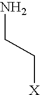 | 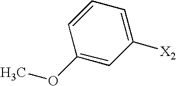 | 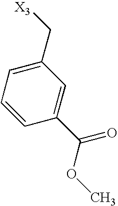 | 3.96 | 399.20 |
| 52 | 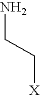 | 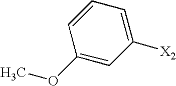 | 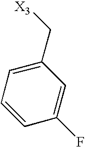 | 3.98 | 359.21 |
| 53 | 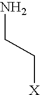 | 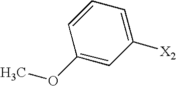 | 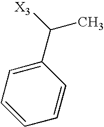 | 4.02 | 355.24 |
| 54 | 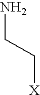 | 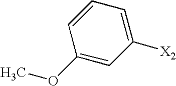 | 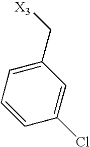 | 4.20 | 375.18 |
| 55 | 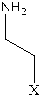 | 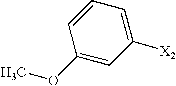 | 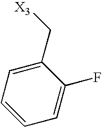 | 3.90 | 359.20 |
| 56 | 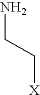 | 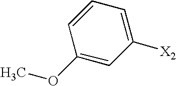 | 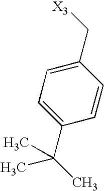 | 4.83 | 397.26 |
| 57 | 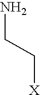 | 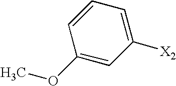 | 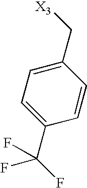 | 4.49 | 409.17 |
| 58 | 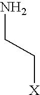 | 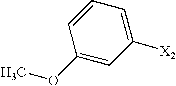 | 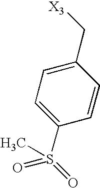 | 3.54 | 419.23 |
| 59 | 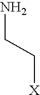 | 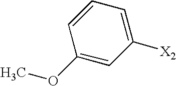 | 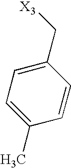 | 4.14 | 355.24 |
| 60 | 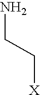 | 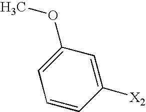 | 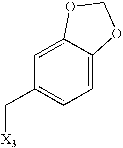 | 7.57 | 385.23 |
| 61 | 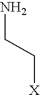 | 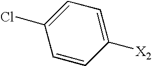 | 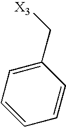 | 4.09 | 345.08 |
| 62 | 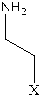 | 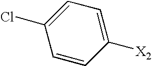 | 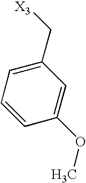 | 4.16 | 375.05 |
| 63 | 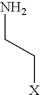 | 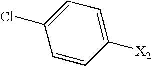 | 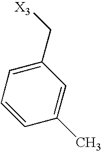 | 4.31 | 359.06 |
| 64 | 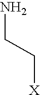 | 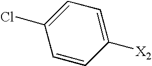 | 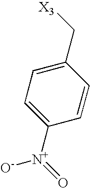 | 4.23 | 390.03 |
| 65 | 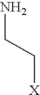 | 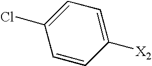 | 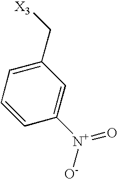 | 4.19 | 390.04 |
| 66 | 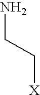 | 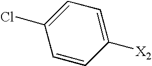 | 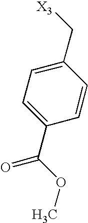 | 4.20 | 403.19 |
| 67 | 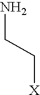 | 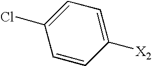 | 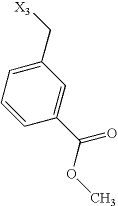 | 4.17 | 403.02 |
| 68 | 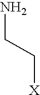 | 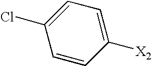 | 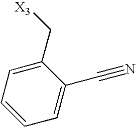 | 4.02 | 370.06 |
| 69 | 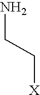 | 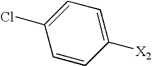 | 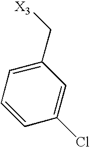 | 4.43 | 379.16 |
| 70 | 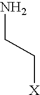 | 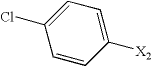 | 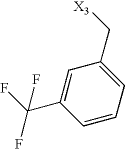 | 4.62 | 413.00 |
| 71 | 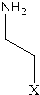 | 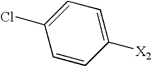 | 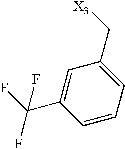 | 4.03 | 370.06 |
| 72 | 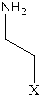 | 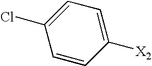 | 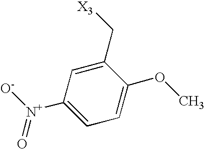 | 4.25 | 420.05 |
| 73 | 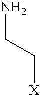 | 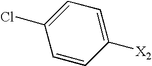 | 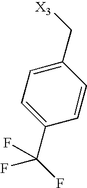 | 4.68 | 413.00 |
| 74 | 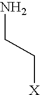 | 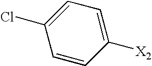 | 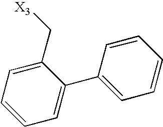 | 4.83 | 421.07 |
| 75 | 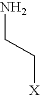 | 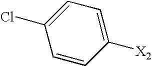 | 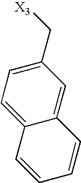 | 4.59 | 395.04 |
| 76 | 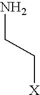 | 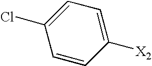 | 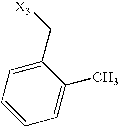 | 4.26 | 359.06 |
| 77 | 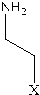 | 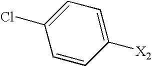 | 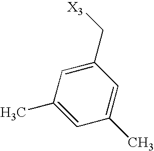 | 4.55 | 373.07 |
| 78 | 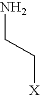 | 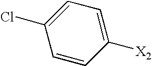 | 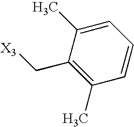 | 4.40 | 377.10 |
| 79 | 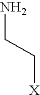 | 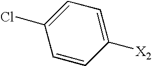 | 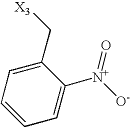 | 4.12 | 390.04 |
| 80 | 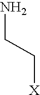 | 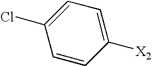 | 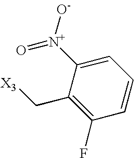 | 4.08 | 407.99 |
| 81 | 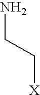 | 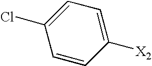 | 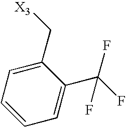 | 4.54 | 413.17 |
| 82 | 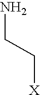 | 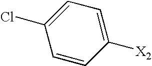 | 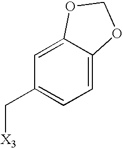 | 7.74 | 389.16 |
| 83 | 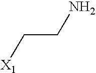 | 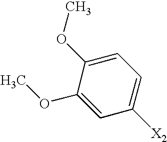 | 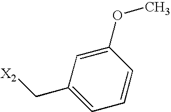 | 6.55 | 401.32 |
| 84 | 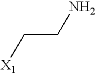 | 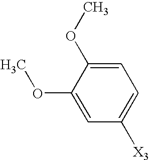 | 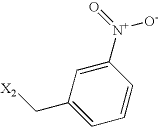 | 6.62 | 416.31 |
| 85 | 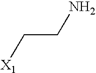 | 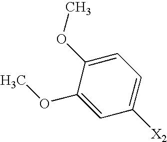 | 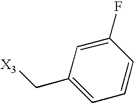 | 6.70 | 389.20 |
| 86 | 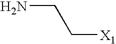 | 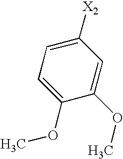 | 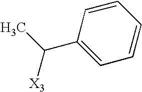 | 6.64 | 385.37 |
| 87 | 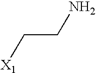 | 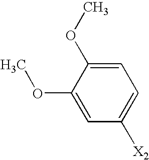 | 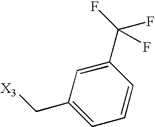 | 7.11 | 439.21 |
| 88 | 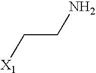 | 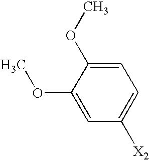 | 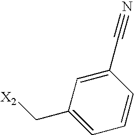 | 6.47 | 396.30 |
| 89 | 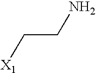 | 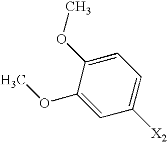 | 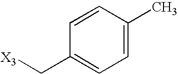 | 6.79 | 385.29 |
| 90 | 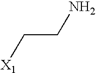 | 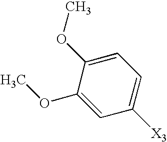 | 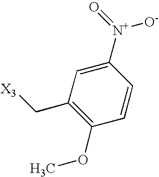 | 6.68 | 446.32 |
| 91 | 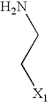 | 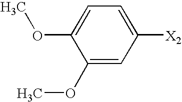 | 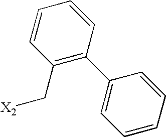 | 7.21 | 447.33 |
| 92 | 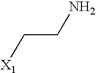 | 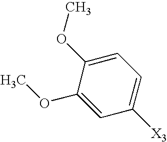 | 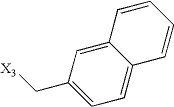 | 6.96 | 421.32 |
| 93 | 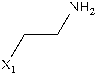 | 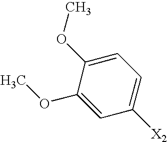 | 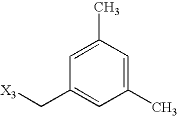 | 6.97 | 399.37 |
| 94 | 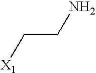 | 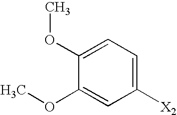 | 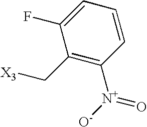 | 6.57 | 434.30 |
| 95 | 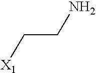 | 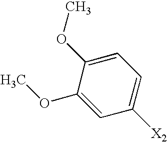 | 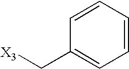 | 6.73 | 371.28 |
| 96 | 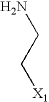 | 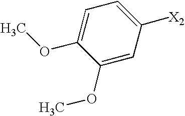 | 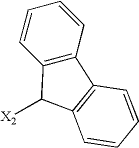 | 7.23 | 445.36 |
| 97 | 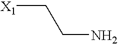 | 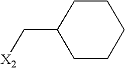 | 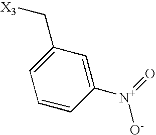 | 6.99 | 376.33 |
| 98 | 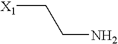 | 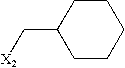 | 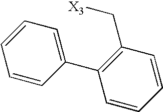 | 7.65 | 407.36 |
| 99 | 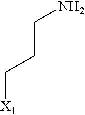 | 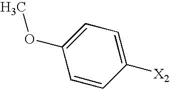 | 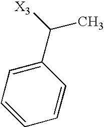 | 4.18 | 369.31 |
| 100 | 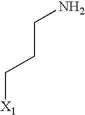 | 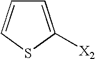 | 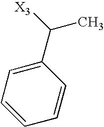 | 4.07 | 345.27 |
| 101 | 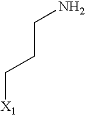 | 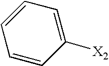 | 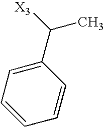 | 4.08 | 339.31 |
| 102 | 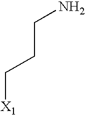 | 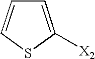 | 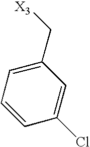 | 4.21 | 365.21 |
| 103 | 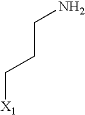 | 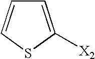 | 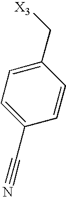 | 3.82 | 356.23 |
| 104 | 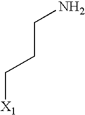 | 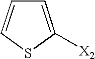 | 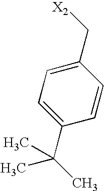 | 4.87 | 387.26 |
| 105 | 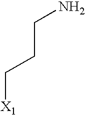 | 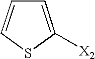 | 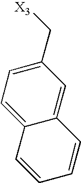 | 4.45 | 381.23 |
| 106 | 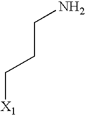 | 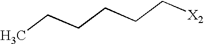 | 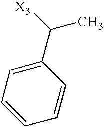 | 4.61 | 347.36 |
| 107 | 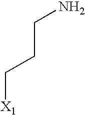 | 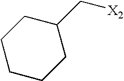 | 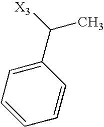 | 4.55 | 359.34 |
| 108 | 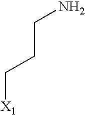 | 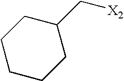 | 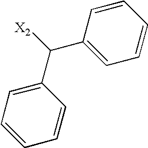 | 5.01 | 421.33 |
| 109 | 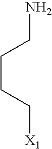 | 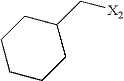 | 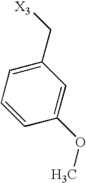 | 3.54 | 383.19 |
| 110 | 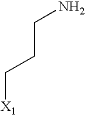 | 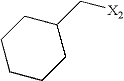 | 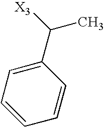 | 4.23 | 353.31 |
| 111 | 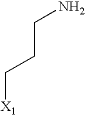 | 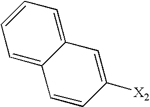 | 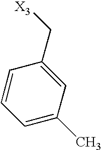 | 4.65 | 389.27 |
| 112 | 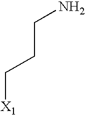 | 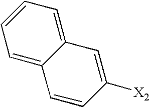 | 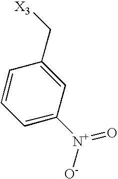 | 4.51 | 420.24 |
| 113 | 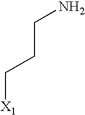 | 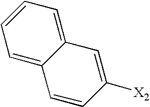 | 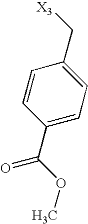 | 4.47 | 433.25 |
| 114 | 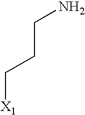 | 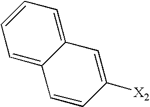 | 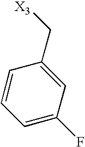 | 4.53 | 393.24 |
| 115 | 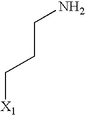 | 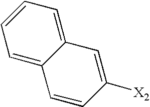 | 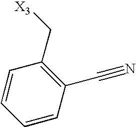 | 4.33 | 400.25 |
| 116 | 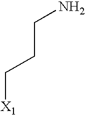 | 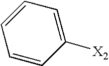 | 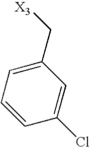 | 4.22 | 359.22 |
| 117 | 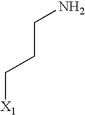 | 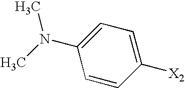 | 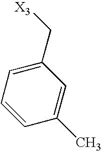 | 4.34 | 382.30 |
| 118 | 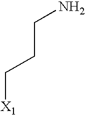 | 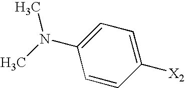 | 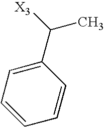 | 4.27 | 382.30 |
| 119 | 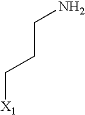 | 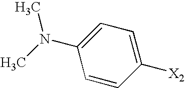 | 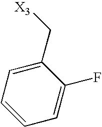 | 4.14 | 386.29 |
| 120 | 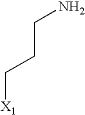 | 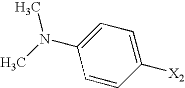 | 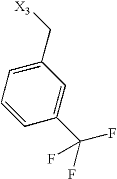 | 4.66 | 436.29 |
| 121 | 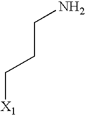 | 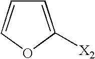 | 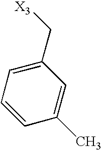 | 4.13 | 329.29 |
| 122 | 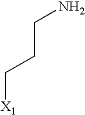 | 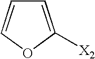 | 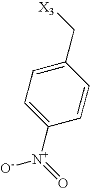 | 3.96 | 360.27 |
| 123 | 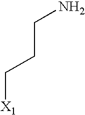 | 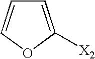 | 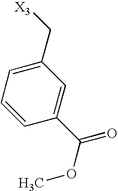 | 3.94 | 373.26 |
| 124 | 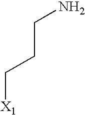 | 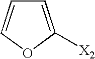 | 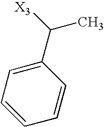 | 4.04 | 329.29 |
| 125 | 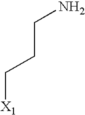 | 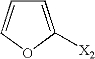 | 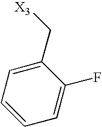 | 3.91 | 333.27 |
| 126 | 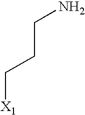 | 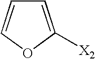 | 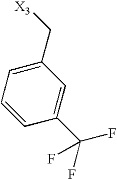 | 4.47 | 383.24 |
| 127 | 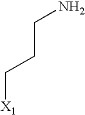 | 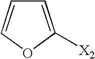 | 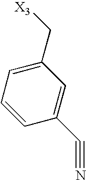 | 3.78 | 340.29 |
| 128 | 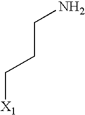 | 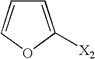 | 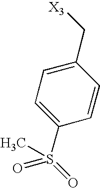 | 3.47 | 393.23 |
| 129 | 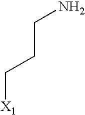 | 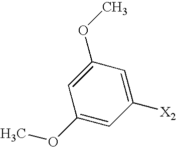 | 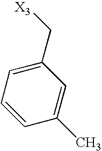 | 4.45 | 399.31 |
| 130 | 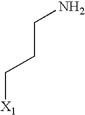 | 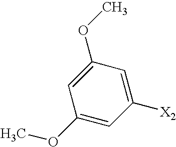 | 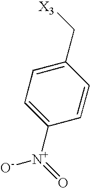 | 4.35 | 430.30 |
| 131 | 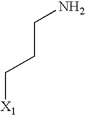 | 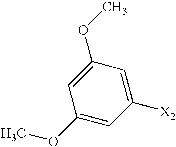 | 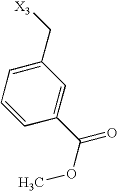 | 4.29 | 443.32 |
| 132 | 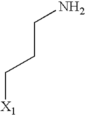 | 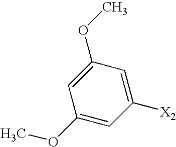 | 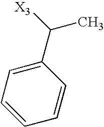 | 4.37 | 399.31 |
| 133 | 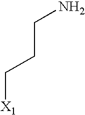 | 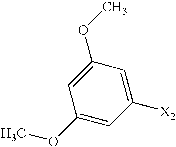 | 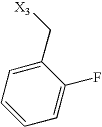 | 4.26 | 403.27 |
| 134 | 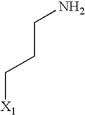 | 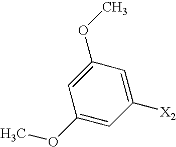 | 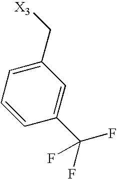 | 4.76 | 453.29 |
| 135 | 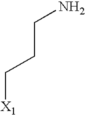 | 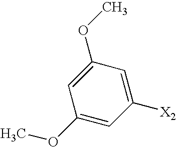 | 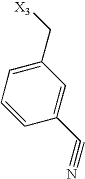 | 4.16 | 410.28 |
| 136 | 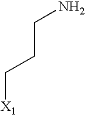 | 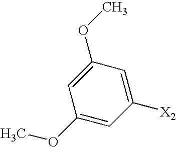 | 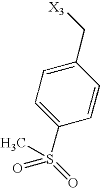 | 3.90 | 463.28 |
| 137 | 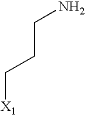 | 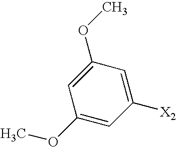 | 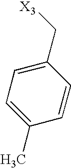 | 4.48 | 399.31 |
| 138 | 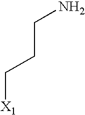 | 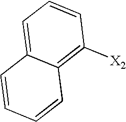 | 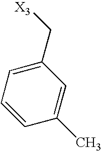 | 4.57 | 389.31 |
| 139 | 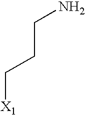 | 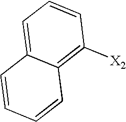 | 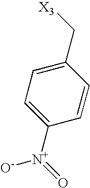 | 4.46 | 420.28 |
| 140 | 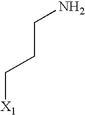 | 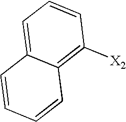 | 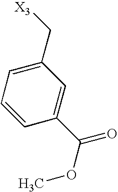 | 4.40 | 433.29 |
| 141 | 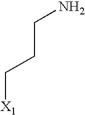 | 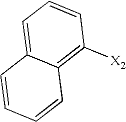 | 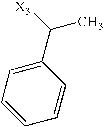 | 4.51 | 389.31 |
| 142 | 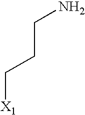 | 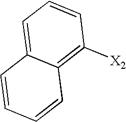 | 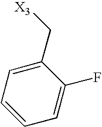 | 4.39 | 393.28 |
| 143 | 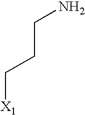 | 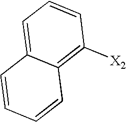 | 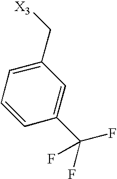 | 4.87 | 443.30 |
| 144 | 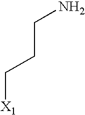 | 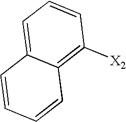 | 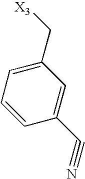 | 4.28 | 400.28 |
| 145 | 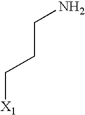 | 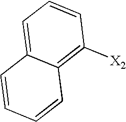 | 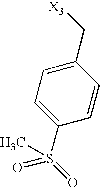 | 4.01 | 453.26 |
| 146 | 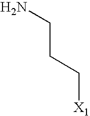 | 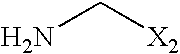 | 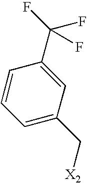 | 6.11 | 346.28 |
| 147 | 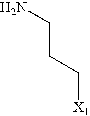 | 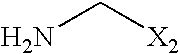 | 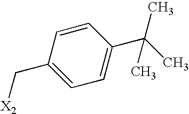 | 6.50 | 334.35 |
| 148 | 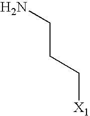 | 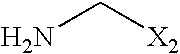 | 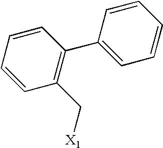 | 6.37 | 354.30 |
| 149 | 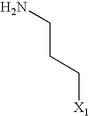 | 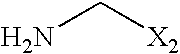 | 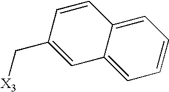 | 6.17 | 328.30 |
| 150 | 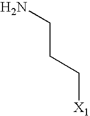 | 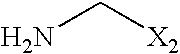 | 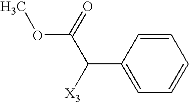 | 5.73 | 336.28 |
| 151 | 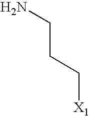 | 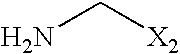 | 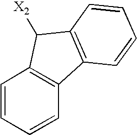 | 6.30 | 352.30 |
| 152 | 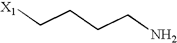 | 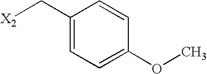 | 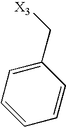 | 3.52 | 383.19 |
| 153 | 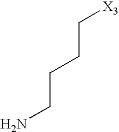 | 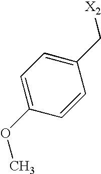 | 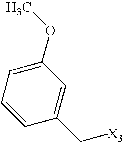 | 3.58 | 413.18 |
| 154 | 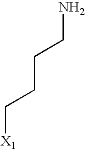 | 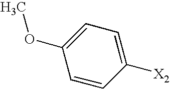 | 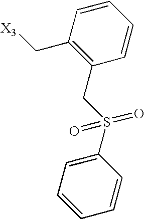 | 7.99 | 523.30 |
| 155 | 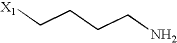 | 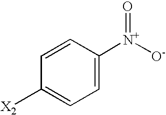 | 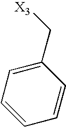 | 3.60 | 384.15 |
| 156 | 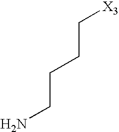 | 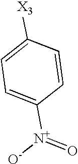 | 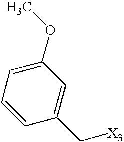 | 3.66 | 414.14 |
| 157 | 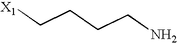 | 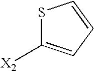 | 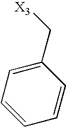 | 3.35 | 345.16 |
| 158 | 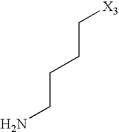 | 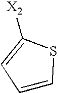 | 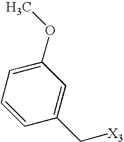 | 3.40 | 375.14 |
| 159 | 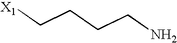 | 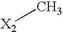 | 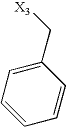 | 2.74 | 277.19 |
| 160 | 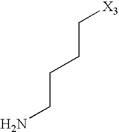 | 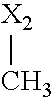 | 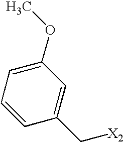 | 2.84 | 307.18 |
| 161 | 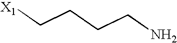 | 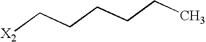 | 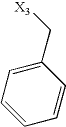 | 3.93 | 347.26 |
| 162 | 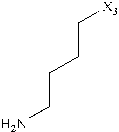 | 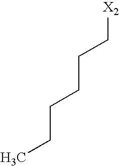 | 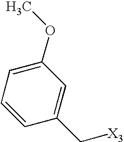 | 3.94 | 377.25 |
| 163 | 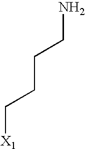 | 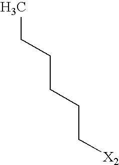 | 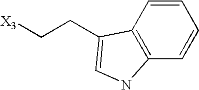 | 8.04 | 400.37 |
| 164 | 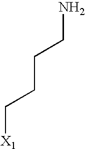 | 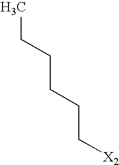 | 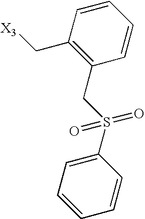 | 8.25 | 501.33 |
| 165 | 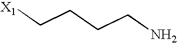 | 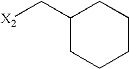 | 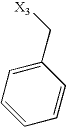 | 3.85 | 359.25 |
| 166 | 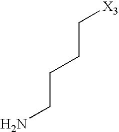 | 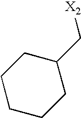 | 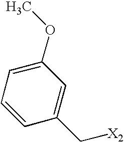 | 3.87 | 389.25 |
| 167 | 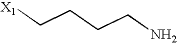 | 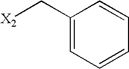 | 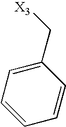 | 3.50 | 353.21 |
| 168 | 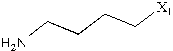 | 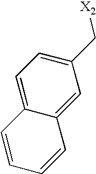 | 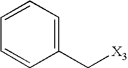 | 3.94 | 403.18 |
| 169 | 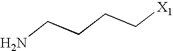 | 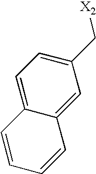 | 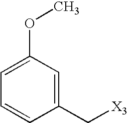 | 3.98 | 433.21 |
| 170 | 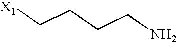 | 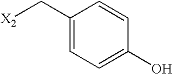 | 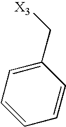 | 3.04 | 369.20 |
| 171 | 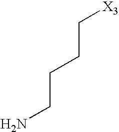 | 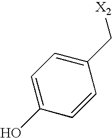 | 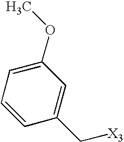 | 3.12 | 399.18 |
| 172 | 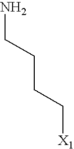 | 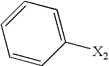 | 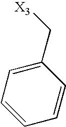 | 3.90 | 339.20 |
| 173 | 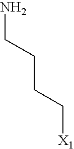 | 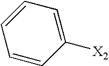 | 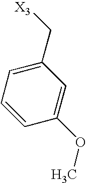 | 4.07 | 369.16 |
| 174 | 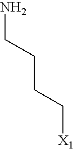 | 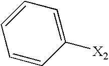 | 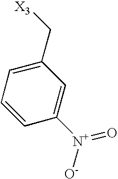 | 4.08 | 384.10 |
| 175 | 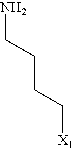 | 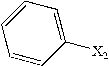 | 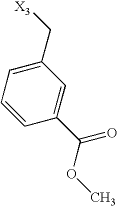 | 4.08 | 397.11 |
| 176 | 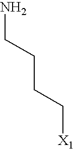 | 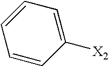 | 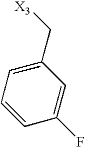 | 4.12 | 357.14 |
| 177 | 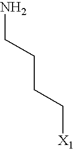 | 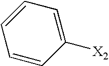 | 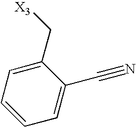 | 3.90 | 364.13 |
| 178 | 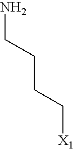 | 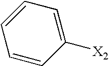 | 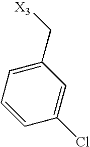 | 4.32 | 373.08 |
| 179 | 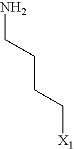 | 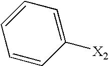 | 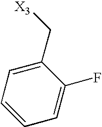 | 4.06 | 357.15 |
| 180 | 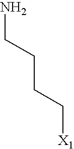 | 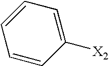 | 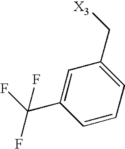 | 4.54 | 407.25 |
| 181 | 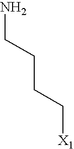 | 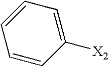 | 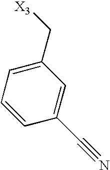 | 3.94 | 364.30 |
| 182 | 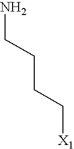 | 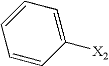 | 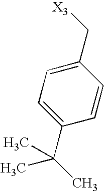 | 4.94 | 395.17 |
| 183 | 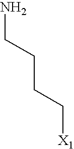 | 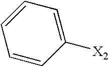 | 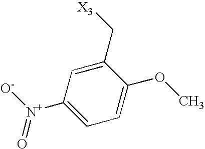 | 4.14 | 414.09 |
| 184 | 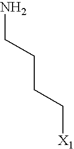 | 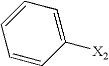 | 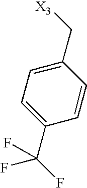 | 4.58 | 407.08 |
| 185 | 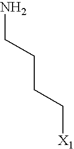 | 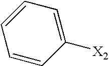 | 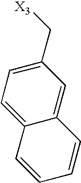 | 4.51 | 389.12 |
| 186 | 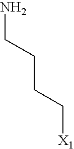 | 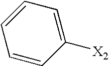 | 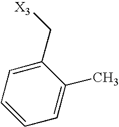 | 4.22 | 353.17 |
| 187 | 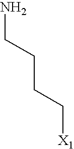 | 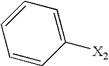 | 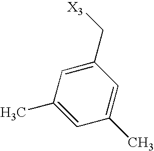 | 4.51 | 367.17 |
| 188 | 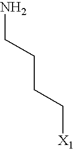 | 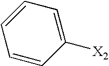 | 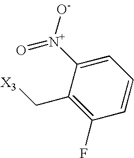 | 4.00 | 402.06 |
| 189 | 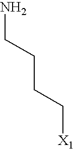 | 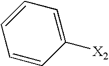 | 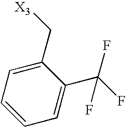 | 4.47 | 407.25 |
| 190 | 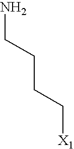 | 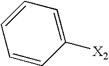 | 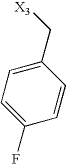 | 4.14 | 357.29 |
| 191 | 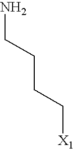 | 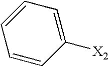 | 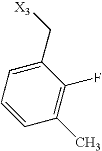 | 4.31 | 371.14 |
| 192 | 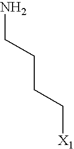 | 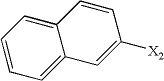 | 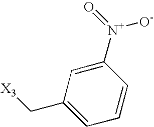 | 7.96 | 433.90 |
| 193 | 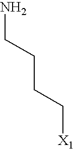 | 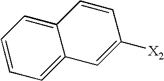 | 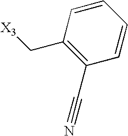 | 7.80 | 413.93 |
| 194 | 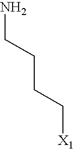 | 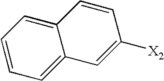 | 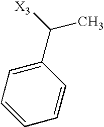 | 8.01 | 402.95 |
| 195 | 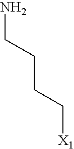 | 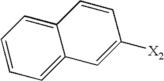 | 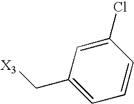 | 8.15 | 422.88 |
| 196 | 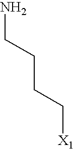 | 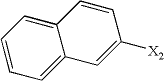 | 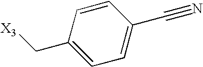 | 7.83 | 413.93 |
| 197 | 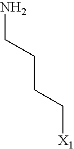 | 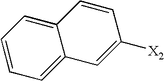 | 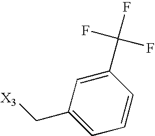 | 8.28 | 456.90 |
| 198 | 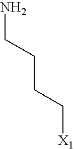 | 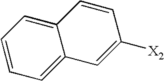 | 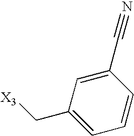 | 7.83 | 413.92 |
| 199 | 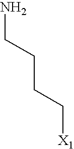 | 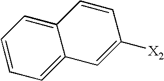 | 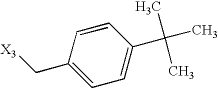 | 8.57 | 445.02 |
| 200 | 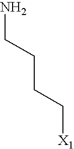 | 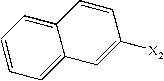 | 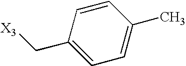 | 8.03 | 403.02 |
| 201 | 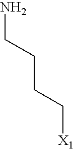 | 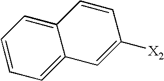 | 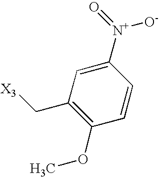 | 7.99 | 463.90 |
| 202 | 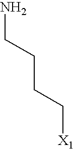 | 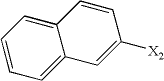 | 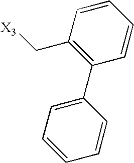 | 8.51 | 464.93 |
| 203 | 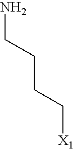 | 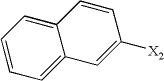 | 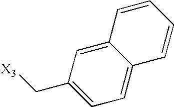 | 8.25 | 438.94 |
| 204 | 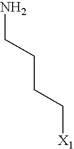 | 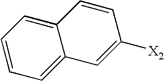 | 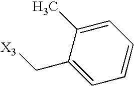 | 8.04 | 402.96 |
| 205 | 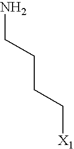 | 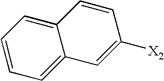 | 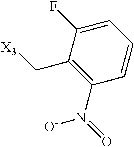 | 7.89 | 451.88 |
| 206 | 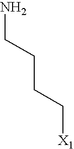 | 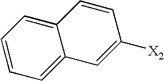 | 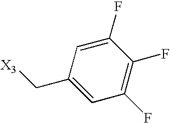 | 8.13 | 442.95 |
| 207 | 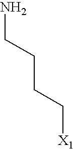 | 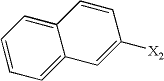 | 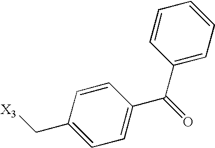 | 8.23 | 492.96 |
| 208 | 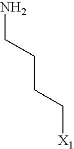 | 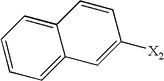 | 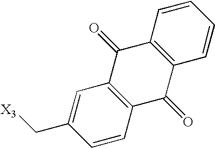 | 8.33 | 518.86 |
| 209 | 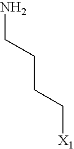 | 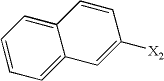 | 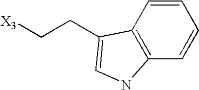 | 8.20 | 442.20 |
| 210 | 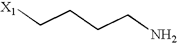 | 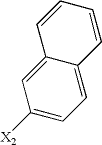 | 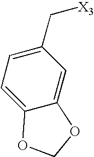 | 8.03 | 433.25 |
| 211 | 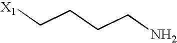 | 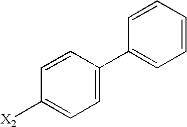 | 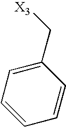 | 4.22 | 415.18 |
| 212 | 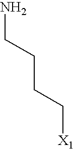 | 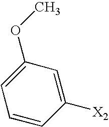 | 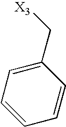 | 4.16 | 369.24 |
| 213 | 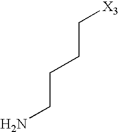 | 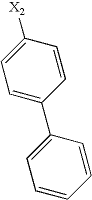 | 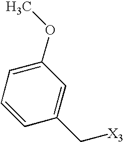 | 4.24 | 445.21 |
| 214 | 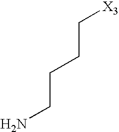 | 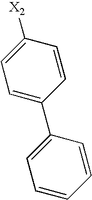 | 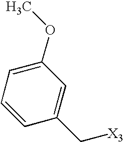 | 8.19 | 444.94 |
| 215 | 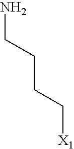 | 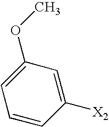 | 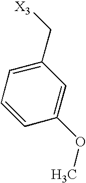 | 4.21 | 399.23 |
| 216 | 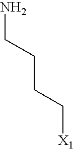 | 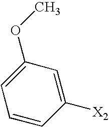 | 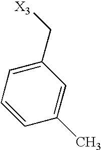 | 4.38 | 383.23 |
| 217 | 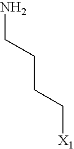 | 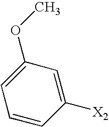 | 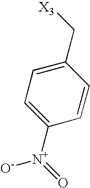 | 4.25 | 414.20 |
| 218 | 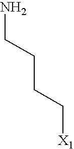 | 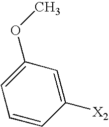 | 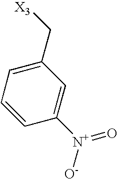 | 4.20 | 414.19 |
| 219 | 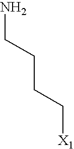 | 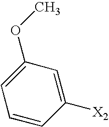 | 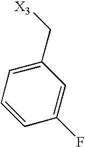 | 4.24 | 387.20 |
| 220 | 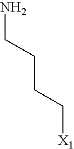 | 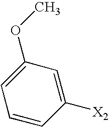 | 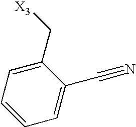 | 4.05 | 394.22 |
| 221 | 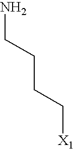 | 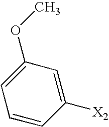 | 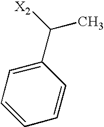 | 4.31 | 383.23 |
| 222 | 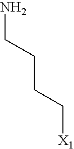 | 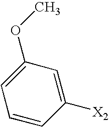 | 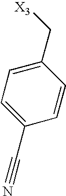 | 4.06 | 394.22 |
| 223 | 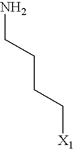 | 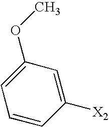 | 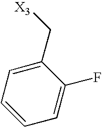 | 4.19 | 387.20 |
| 224 | 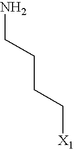 | 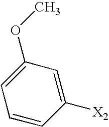 | 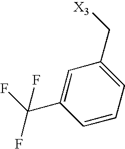 | 4.62 | 437.21 |
| 225 | 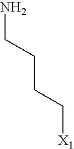 | 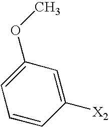 | 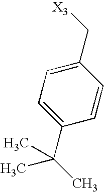 | 5.01 | 425.28 |
| 226 | 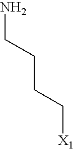 | 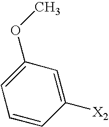 | 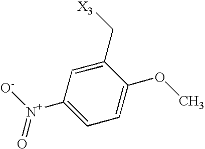 | 4.28 | 444.24 |
| 227 | 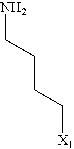 | 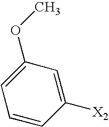 | 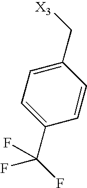 | 4.68 | 437.21 |
| 228 | 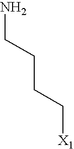 | 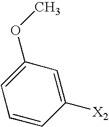 | 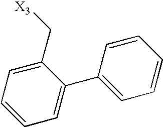 | 4.84 | 445.27 |
| 229 | 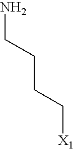 | 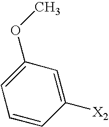 | 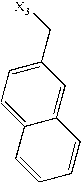 | 4.60 | 419.25 |
| 230 | 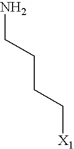 | 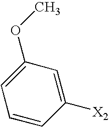 | 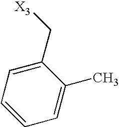 | 4.32 | 383.23 |
| 231 | 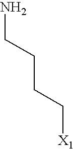 | 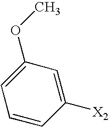 | 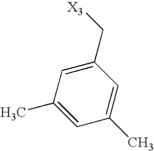 | 4.60 | 397.24 |
| 232 | 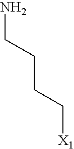 | 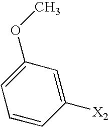 | 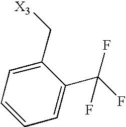 | 4.56 | 437.21 |
| 233 | 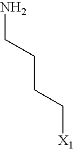 | 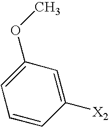 | 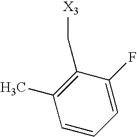 | 4.42 | 401.21 |
| 234 | 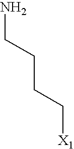 | 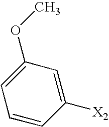 | 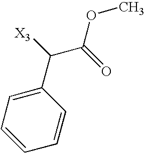 | 4.28 | 427.22 |
| 235 | 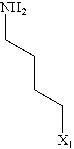 | 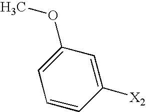 | 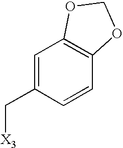 | 7.73 | 413.24 |
| 236 | 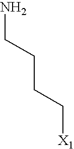 | 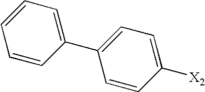 | 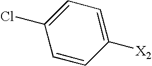 | 8.26 | 459.27 |
| 237 | 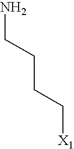 | 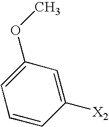 | 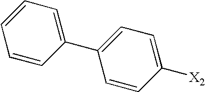 | 4.84 | 443.26 |
| 238 | 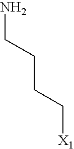 | 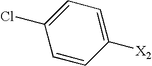 | 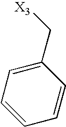 | 4.34 | 373.18 |
| 239 | 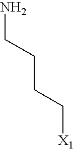 | 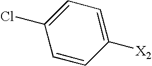 | 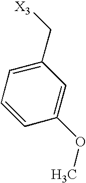 | 4.39 | 403.16 |
| 240 | 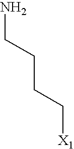 | 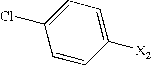 | 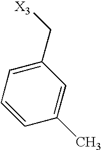 | 4.56 | 387.17 |
| 241 | 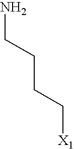 | 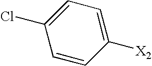 | 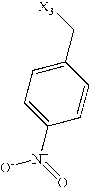 | 4.44 | 418.14 |
| 242 | 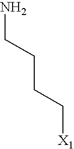 | 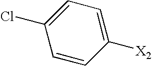 | 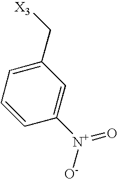 | 4.40 | 418.14 |
| 243 | 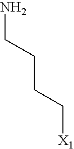 | 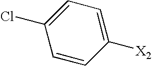 | 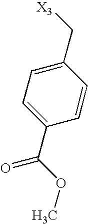 | 4.37 | 431.17 |
| 244 | 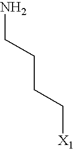 | 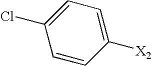 | 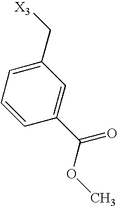 | 4.38 | 431.18 |
| 245 | 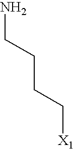 | 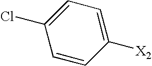 | 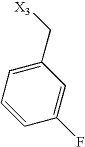 | 4.44 | 391.17 |
| 246 | 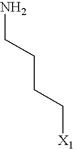 | 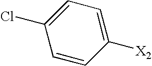 | 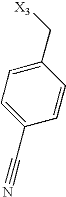 | 4.25 | 398.15 |
| 247 | 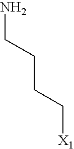 | 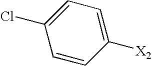 | 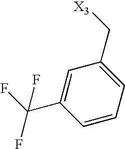 | 4.80 | 441.14 |
| 248 | 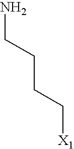 | 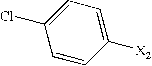 | 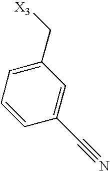 | 4.26 | 398.14 |
| 249 | 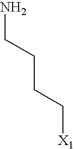 | 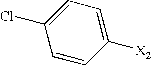 | 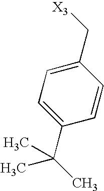 | 5.17 | 429.24 |

| | | | | | |
|---|---|---|---|---|---|
| 250 | 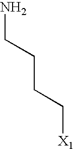 | 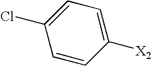 | 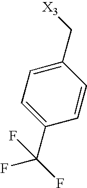 | 4.86 | 441.14 |
| 251 | 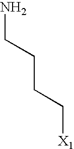 | 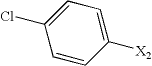 | 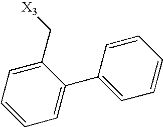 | 4.99 | 449.22 |
| 252 | 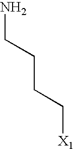 | 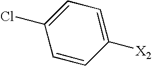 | 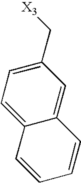 | 4.77 | 423.18 |
| 253 | 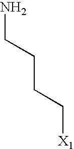 | 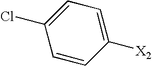 | 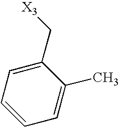 | 4.50 | 387.18 |
| 254 | 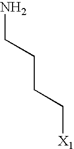 | 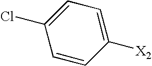 | 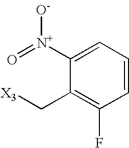 | 4.32 | 436.14 |
| 255 | 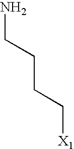 | 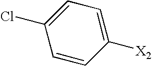 | 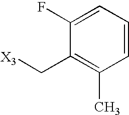 | 4.59 | 405.17 |
| 256 | 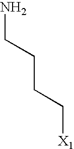 | 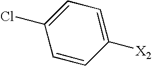 | 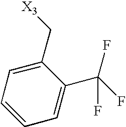 | 4.72 | 441.15 |
| 257 | 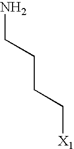 | 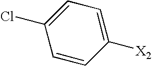 | 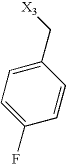 | 4.44 | 391.17 |
| 258 | 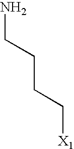 | 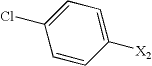 | 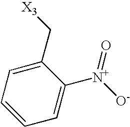 | 4.36 | 418.15 |
| 259 | 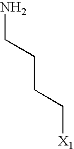 | 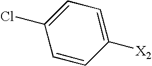 | 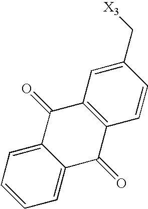 | 4.86 | 503.21 |
| 260 | 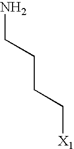 | 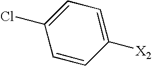 | 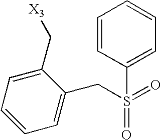 | 4.50 | 448.20 |
| 261 | 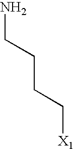 | 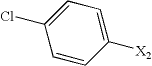 | 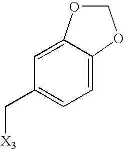 | 7.90 | 417.19 |
| 262 | 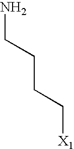 | 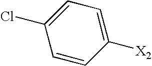 | 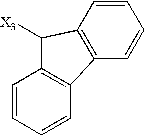 | 5.00 | 447.19 |
| 263 | 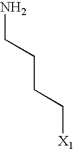 | 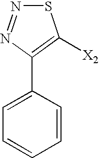 | 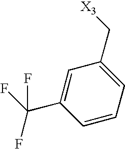 | 4.93 | 491.19 |
| 264 | 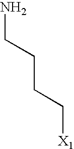 | 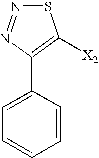 | 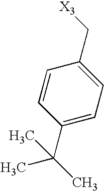 | 5.29 | 479.27 |
| 265 | 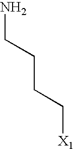 | 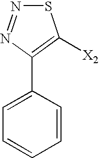 | 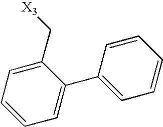 | 5.13 | 499.24 |
| 266 | 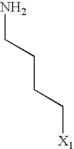 | 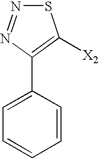 | 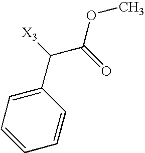 | 4.62 | 481.21 |
| 267 | 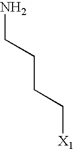 | 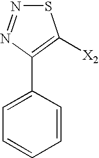 | 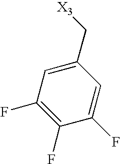 | 4.83 | 477.18 |
| 268 | 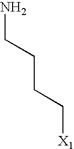 | 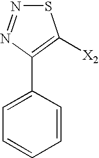 | 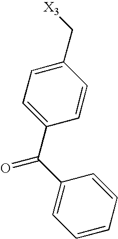 | 4.93 | 527.24 |
| 269 | 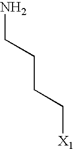 | 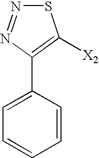 | 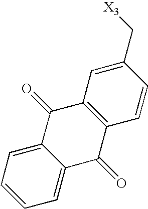 | 4.97 | 553.19 |
| 270 | 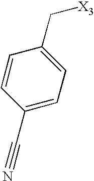 | 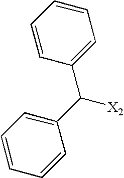 | 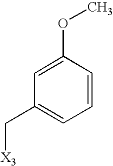 | 8.10 | 458.96 |
| 271 | 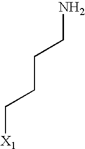 | 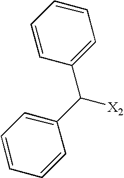 | 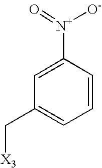 | 8.13 | 473.89 |
| 272 | 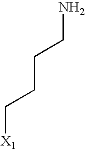 | 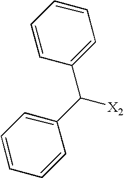 | 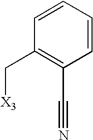 | 7.98 | 453.96 |
| 273 | 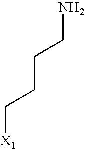 | 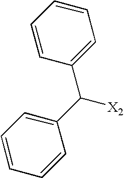 | 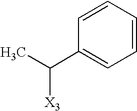 | 8.19 | 442.97 |
| 274 | 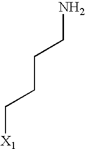 | 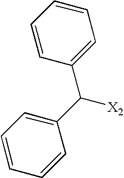 | 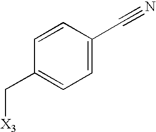 | 7.97 | 453.95 |
| 275 | 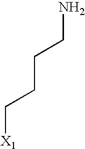 | 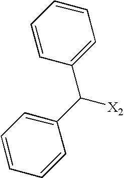 | 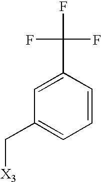 | 8.49 | 496.91 |
| 276 | 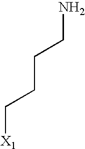 | 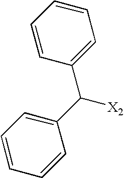 | 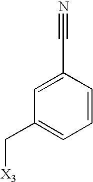 | 7.98 | 453.94 |
| 277 | 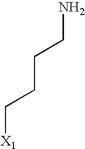 | 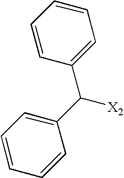 | 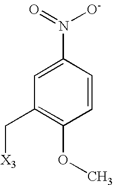 | 8.17 | 503.91 |
| 278 | 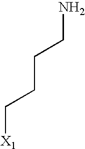 | 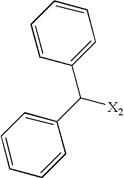 | 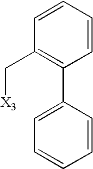 | 8.70 | 504.95 |
| 279 | 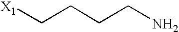 | 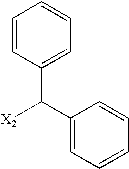 | 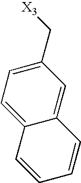 | 8.43 | 478.96 |
| 280 | 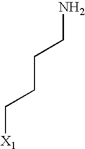 | 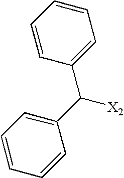 | 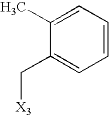 | 8.18 | 442.97 |
| 281 | 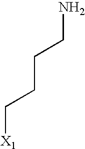 | 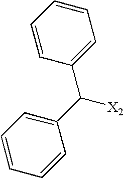 | 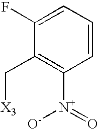 | 8.07 | 491.89 |
| 282 | 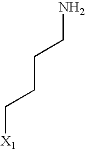 | 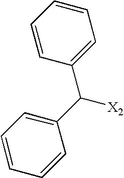 | 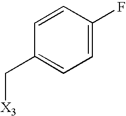 | 8.14 | 446.92 |
| 283 | 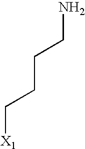 | 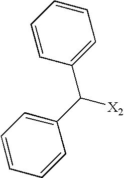 | 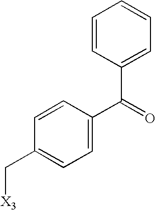 | 8.46 | 532.87 |
| 284 | 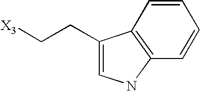 | 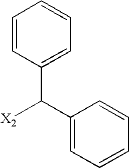 | 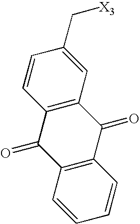 | 8.51 | 558.83 |
| 285 | 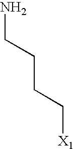 | 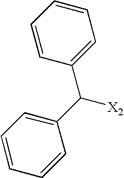 | 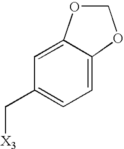 | 8.17 | 473.28 |
| 286 | 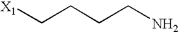 | 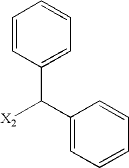 | 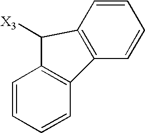 | 8.71 | 502.91 |
| 287 | 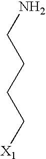 | 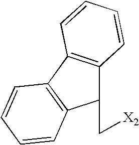 | 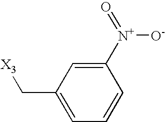 | 8.33 | 485.89 |
| 288 | 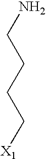 | 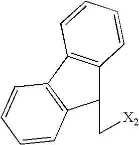 | 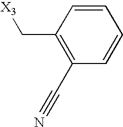 | 8.21 | 465.93 |
| 289 | 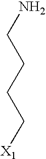 | 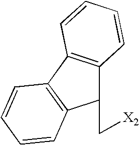 | 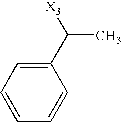 | 8.41 | 454.95 |
| 290 | 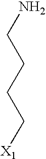 | 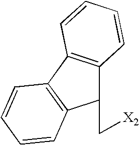 | 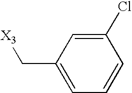 | 8.55 | 474.88 |
| 291 | 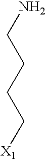 | 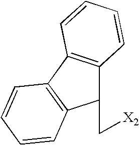 | 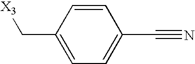 | 8.17 | 465.93 |
| 292 | 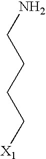 | 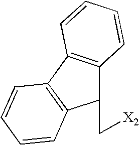 | 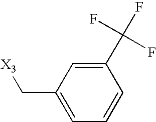 | 8.69 | 511.31 |
| 293 | 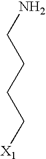 | 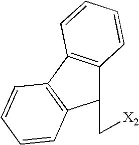 | 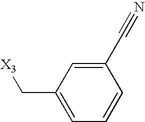 | 8.19 | 465.93 |
| 294 | 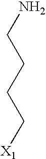 | 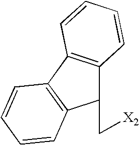 | 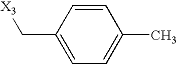 | 8.50 | 455.00 |
| 295 | 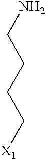 | 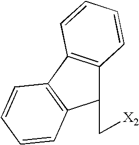 | 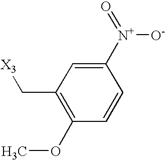 | 8.36 | 515.87 |
| 296 | 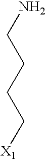 | 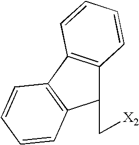 | 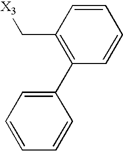 | 8.92 | 516.91 |
| 297 | 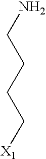 | 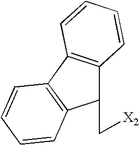 | 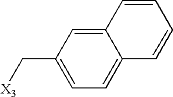 | 8.67 | 490.91 |
| 298 | 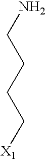 | 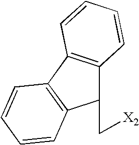 | 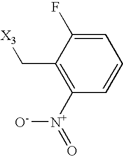 | 8.31 | 503.87 |
| 299 | 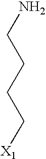 | 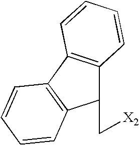 | 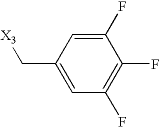 | 8.59 | 494.88 |
| 300 | 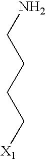 | 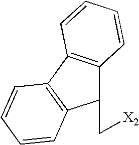 | 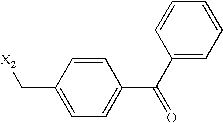 | 8.65 | 544.86 |
| 301 | 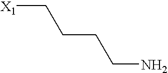 | 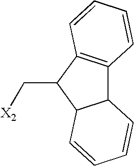 | 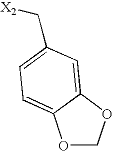 | 8.37 | 485.29 |
| 302 | 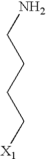 | 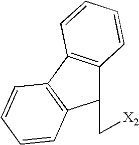 | 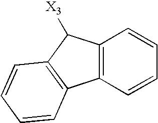 | 8.92 | 514.89 |
| 303 | 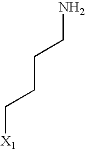 | 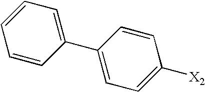 | 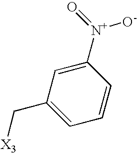 | 8.23 | 459.91 |
| 304 | 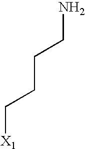 | 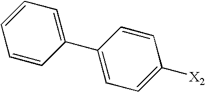 | 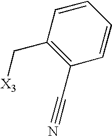 | 8.07 | 439.94 |
| 305 | 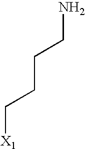 | 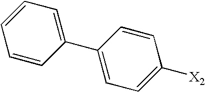 | 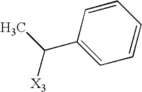 | 8.30 | 428.97 |
| 306 | 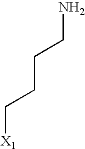 | 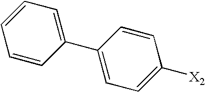 | 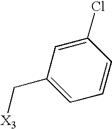 | 8.41 | 448.90 |
| 307 | 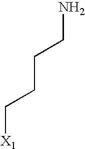 | 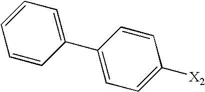 | 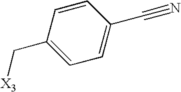 | 8.08 | 439.94 |
| 308 | 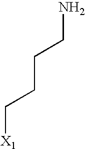 | 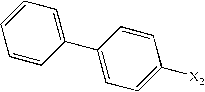 | 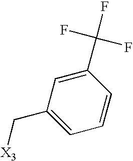 | 8.54 | 482.90 |
| 309 | 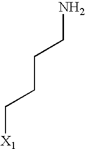 | 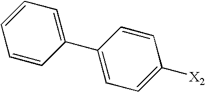 | 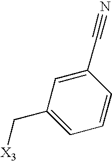 | 8.09 | 439.94 |
| 310 | 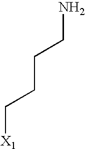 | 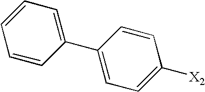 | 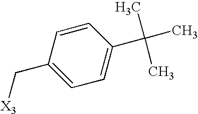 | 8.94 | 470.96 |
| 311 | 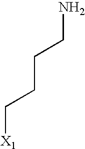 | 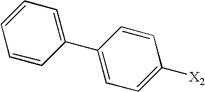 | 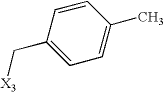 | 8.36 | 428.97 |
| 312 | 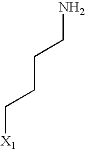 | 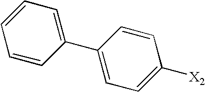 | 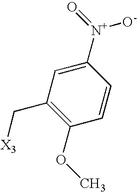 | 8.24 | 489.91 |
| 313 | 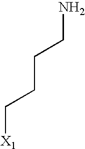 | 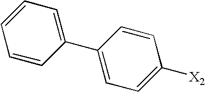 | 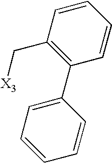 | 8.77 | 490.93 |
| 314 | 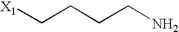 | 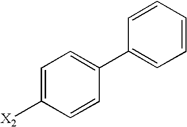 | 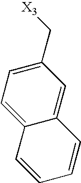 | 8.54 | 464.94 |
| 315 | 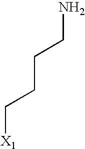 | 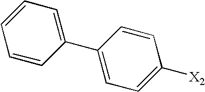 | 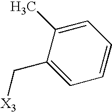 | 8.31 | 428.98 |
| 316 | 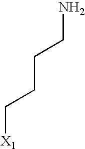 | 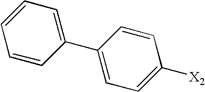 | 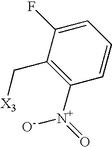 | 8.18 | 477.89 |
| 317 | 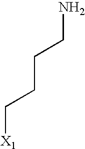 | 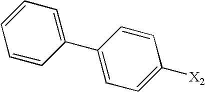 | 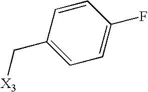 | 8.22 | 432.95 |
| 318 | 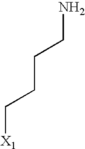 | 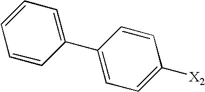 | 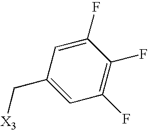 | 8.46 | 468.89 |
| 319 | 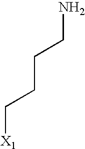 | 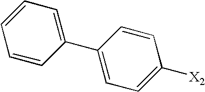 | 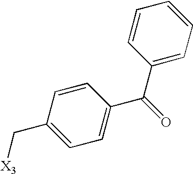 | 8.59 | 518.90 |
| 320 | 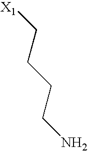 | 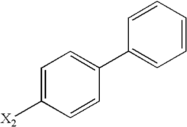 | 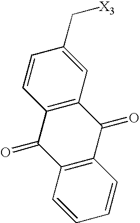 | 8.60 | 544.85 |
| 321 | 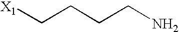 | 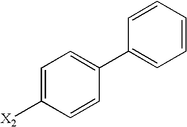 | 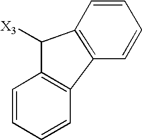 | 8.79 | 488.96 |
| 322 | 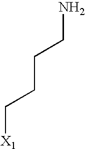 | 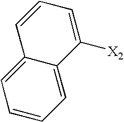 | 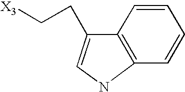 | 8.07 | 442.30 |
| 323 | 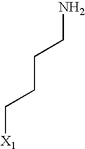 | 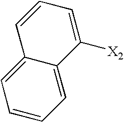 | 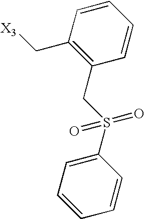 | 8.19 | 543.29 |
| 324 | 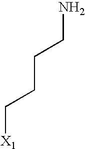 | 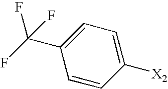 | 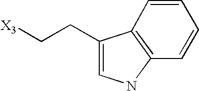 | 8.20 | 460.29 |
| 325 | 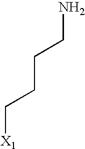 | 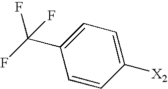 | 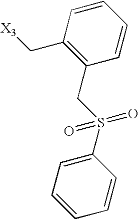 | 8.34 | 561.26 |
| 326 | 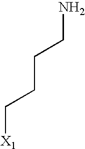 | 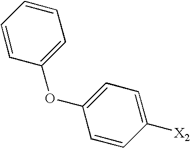 | 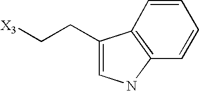 | 8.40 | 484.34 |
| 327 | 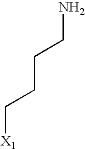 | 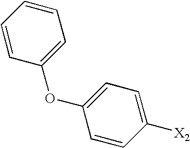 | 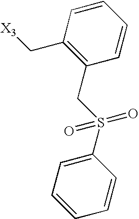 | 8.54 | 585.30 |
| 328 | 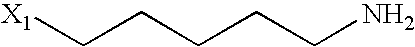 | 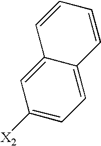 | 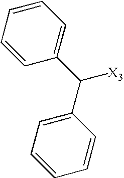 | 8.64 | 479.36 |
| 329 | 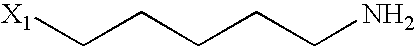 | 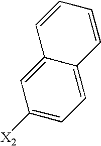 | 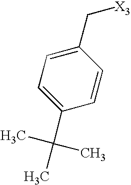 | 8.81 | 459.36 |
| 330 | 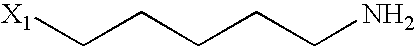 | 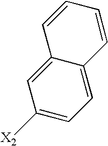 | 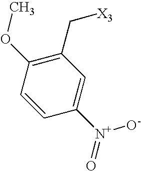 | 8.18 | 478.30 |
| 331 | 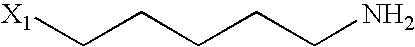 | 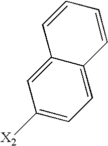 | 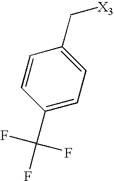 | 8.51 | 471.28 |
| 332 | 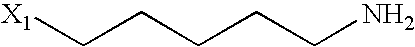 | 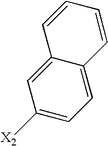 | 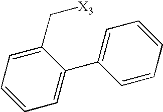 | 8.65 | 479.37 |
| 333 | 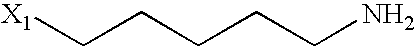 | 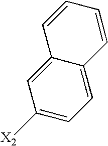 | 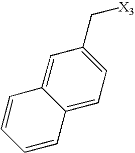 | 8.41 | 453.32 |
| 334 | 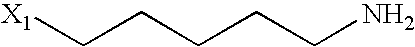 | 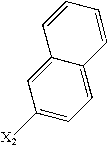 | 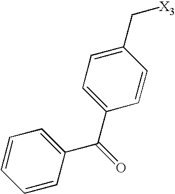 | 8.48 | 507.34 |
| 335 | 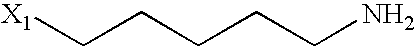 | 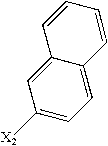 | 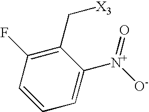 | 8.12 | 466.29 |
| 336 | 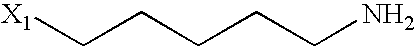 | 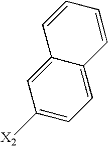 | 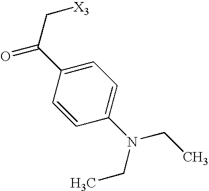 | 8.50 | 502.40 |
| 337 | 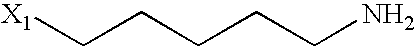 | 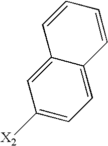 | 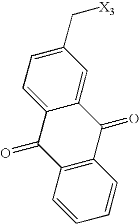 | 8.51 | 533.30 |
| 338 | 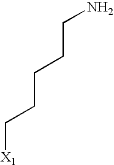 | 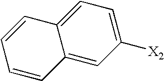 | 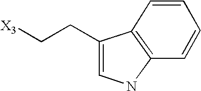 | 8.30 | 456.30 |
| 339 | 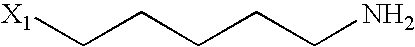 | 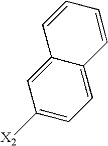 | 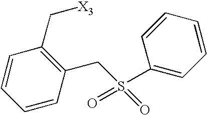 | 8.41 | 557.30 |
| 340 | 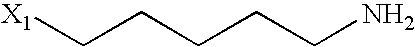 | 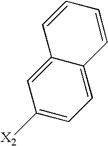 | 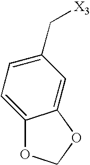 | 8.10 | 447.27 |
| 341 | 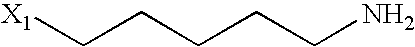 | 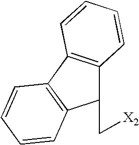 | 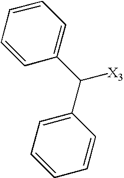 | 8.78 | 493.36 |
| 342 | 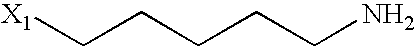 | 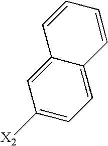 | 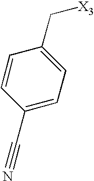 | 8.14 | 442.29 |
| 343 | 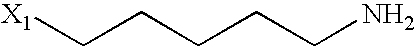 | 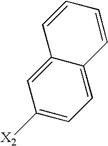 | 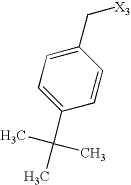 | 8.94 | 473.36 |
| 344 | 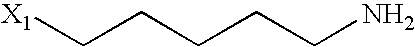 | 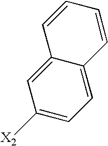 | 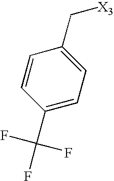 | 8.61 | 485.31 |
| 345 | 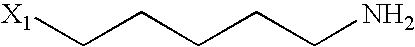 | 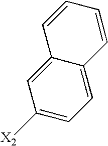 | 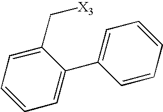 | 8.79 | 493.36 |
| 346 | 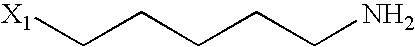 | 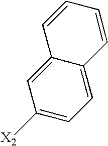 | 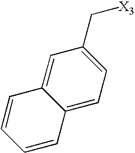 | 8.55 | 467.32 |
| 347 | 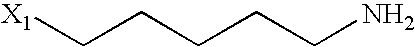 | 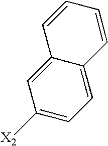 | 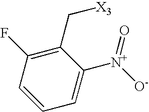 | 8.24 | 480.30 |
| 348 | 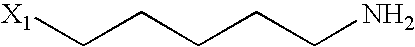 | 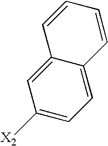 | 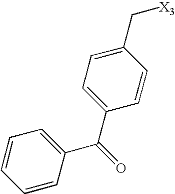 | 8.61 | 521.35 |
| 349 | 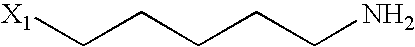 | 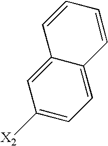 | 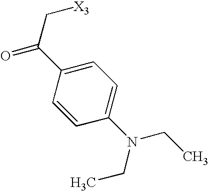 | 8.60 | 516.40 |
| 350 | 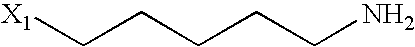 | 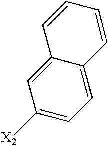 | 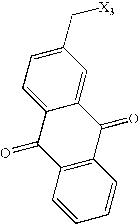 | 8.61 | 547.30 |
| 351 | 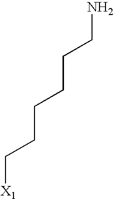 | 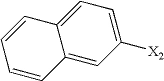 | 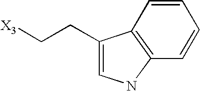 | 8.40 | 470.30 |
| 352 | 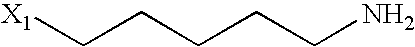 | 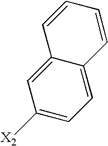 | 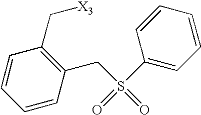 | 8.48 | 571.30 |
| 353 | 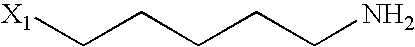 | 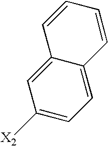 | 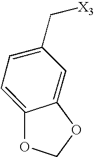 | 8.23 | 461.28 |
| 354 | 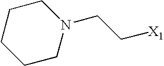 | 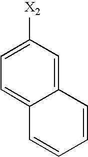 | 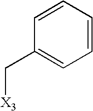 | 7.56 | 429.26 |
| 355 | 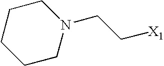 | 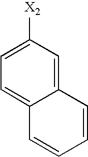 | 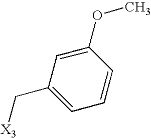 | 7.39 | 459.39 |
| 356 | 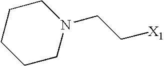 | 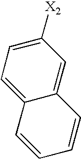 | 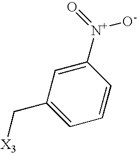 | 7.45 | 474.39 |
| 357 | 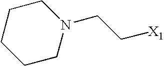 | 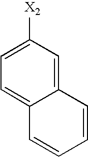 | 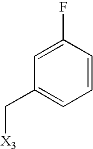 | 7.59 | 447.27 |
| 358 | 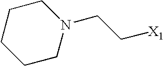 | 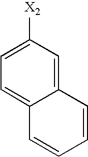 | 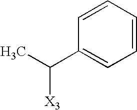 | 7.54 | 443.43 |
| 359 | 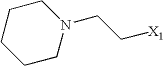 | 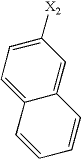 | 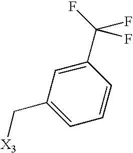 | 8.03 | 497.25 |
| 360 | 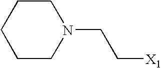 | 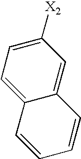 | 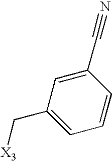 | 7.29 | 454.40 |
| 361 | 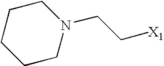 | 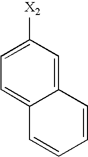 | 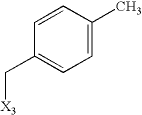 | 7.59 | 443.38 |
| 362 | 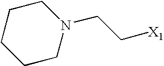 | 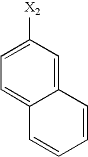 | 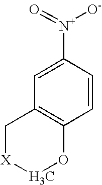 | 7.50 | 504.41 |
| 363 | 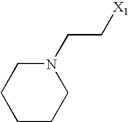 | 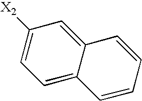 | 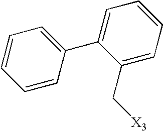 | 8.07 | 505.42 |
| 364 | 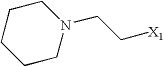 | 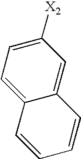 | 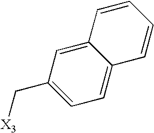 | 7.78 | 479.43 |
| 365 | 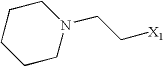 | 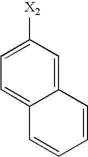 | 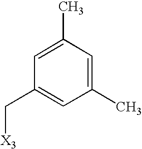 | 7.84 | 457.43 |
| 366 | 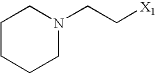 | 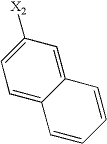 | 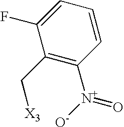 | 7.45 | 492.38 |
| 367 | 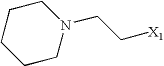 | 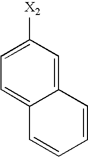 | 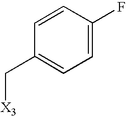 | 7.55 | 447.39 |
| 368 | 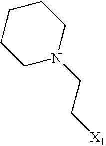 | 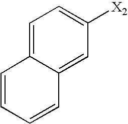 | 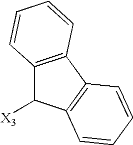 | 8.18 | 503.46 |
| 369 | 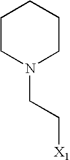 | 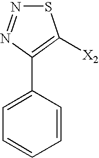 | 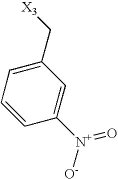 | 4.76 | 508.21 |
| 370 | 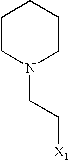 | 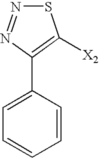 | 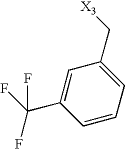 | 5.18 | 531.21 |
| 371 | 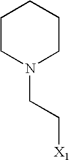 | 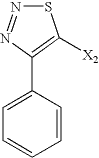 | 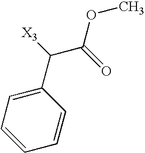 | 4.85 | 521.22 |
| 372 | 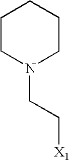 | 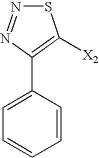 | 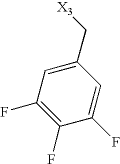 | 5.07 | 517.21 |
| 373 | 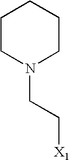 | 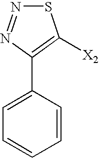 | 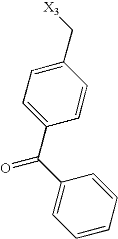 | 5.17 | 567.23 |
| 374 | 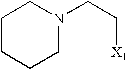 | 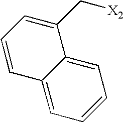 | 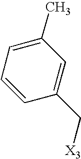 | 7.56 | 457.26 |
| 375 | 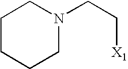 | 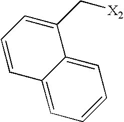 | 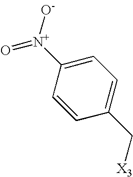 | 10.90 | 488.31 |
| 376 | 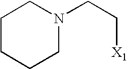 | 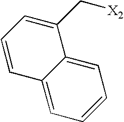 | 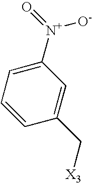 | 7.48 | 488.21 |
| 377 | 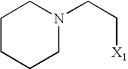 | 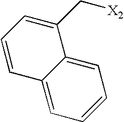 | 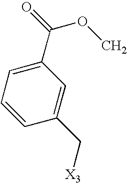 | 7.38 | 501.23 |
| 378 | 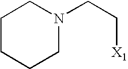 | 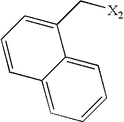 | 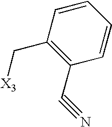 | 12.40 | 465.28 |
| 379 | 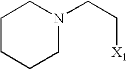 | 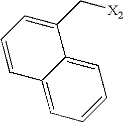 | 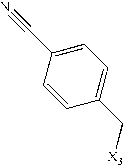 | 7.25 | 468.24 |
| 380 | 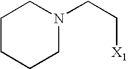 | 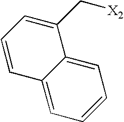 | 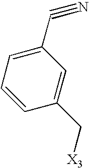 | 7.30 | 468.24 |
| 381 | 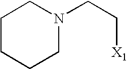 | 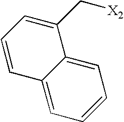 | 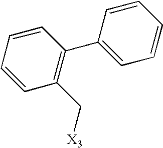 | 8.12 | 519.26 |
| 382 | 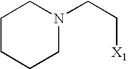 | 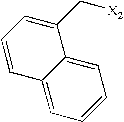 | 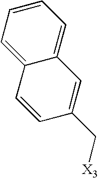 | 7.84 | 493.34 |
| 383 | 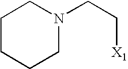 | 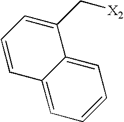 | 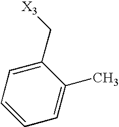 | 7.48 | 457.26 |
| 384 | 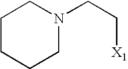 | 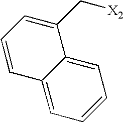 | 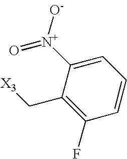 | 7.40 | 506.23 |
| 385 | 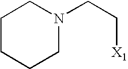 | 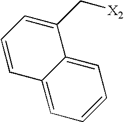 | 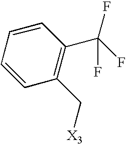 | 11.16 | 511.31 |
| 386 | 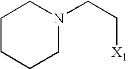 | 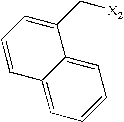 | 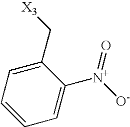 | 7.42 | 488.23 |
| 387 | 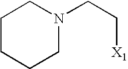 | 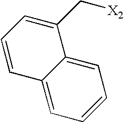 | 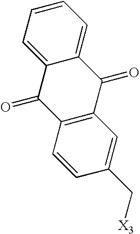 | 7.89 | 573.20 |
| 388 | 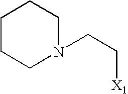 | 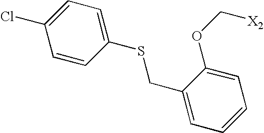 | 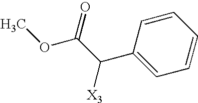 | 8.98 | 623.35 |
| 389 | 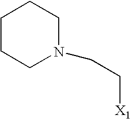 | 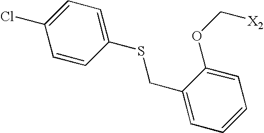 | 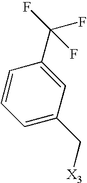 | 9.29 | 633.34 |
| 390 | 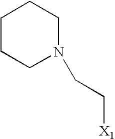 | 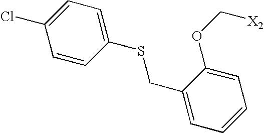 | 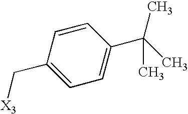 | 9.77 | 621.41 |
| 391 | 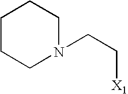 | 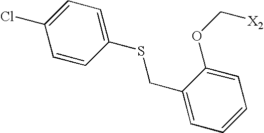 | 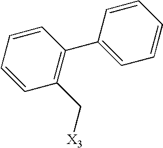 | 9.52 | 641.39 |
| 392 | 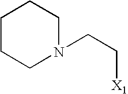 | 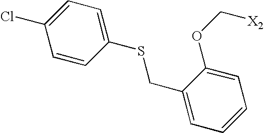 | 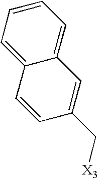 | 9.28 | 615.35 |
| 393 | 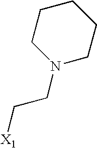 | 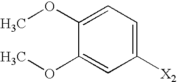 | 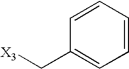 | 6.95 | 439.28 |
| 394 | 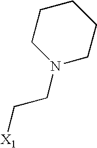 | 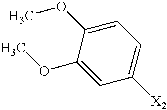 | 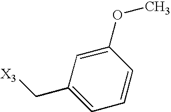 | 6.86 | 469.39 |
| 395 | 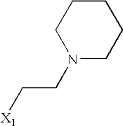 | 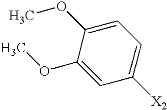 | 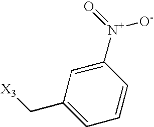 | 6.91 | 484.42 |
| 396 | 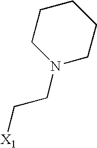 | 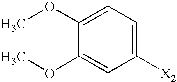 | 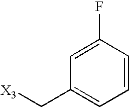 | 7.01 | 457.29 |
| 397 | 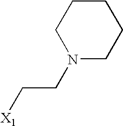 | 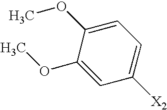 | 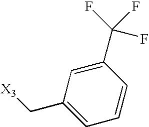 | 7.45 | 507.26 |
| 398 | 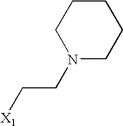 | 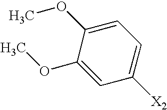 | 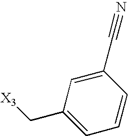 | 6.76 | 464.41 |
| 399 | 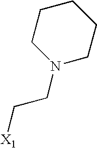 | 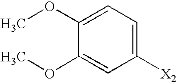 | 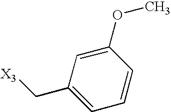 | 7.08 | 453.39 |
| 400 | 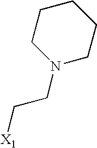 | 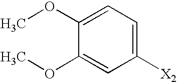 | 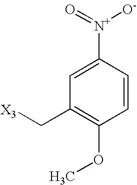 | 7.01 | 514.40 |
| 401 | 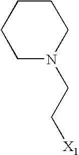 | 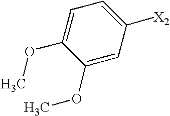 | 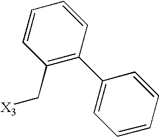 | 7.59 | 515.41 |
| 402 | 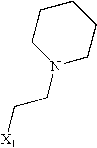 | 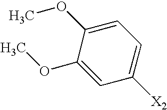 | 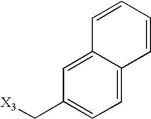 | 7.28 | 489.44 |
| 403 | 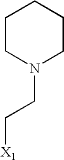 | 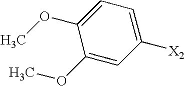 | 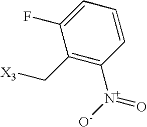 | 6.90 | 502.40 |
| 404 | 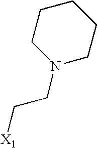 | 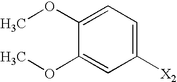 | 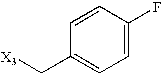 | 6.98 | 457.41 |
| 405 | 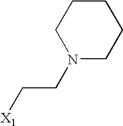 | 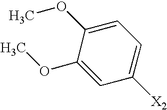 | 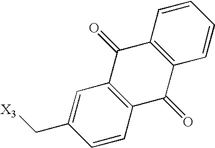 | 7.43 | 569.36 |
| 406 | 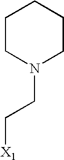 | 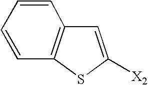 | 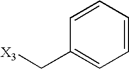 | 7.67 | 435.22 |
| 407 | 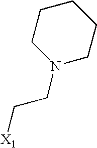 | 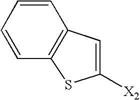 | 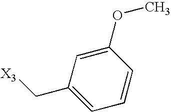 | 7.49 | 465.36 |
| 408 | 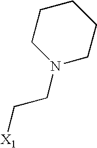 | 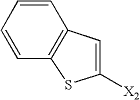 | 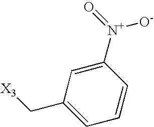 | 7.58 | 480.37 |
| 409 | 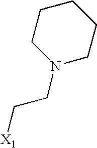 | 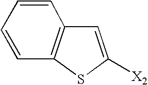 | 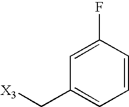 | 7.70 | 453.27 |
| 410 | 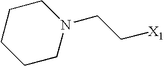 | 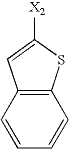 | 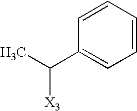 | 7.69 | 449.38 |
| 411 | 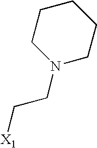 | 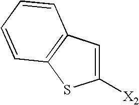 | 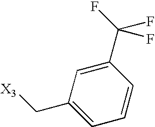 | 8.16 | 503.23 |
| 412 | 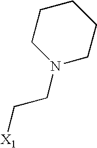 | 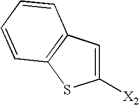 | 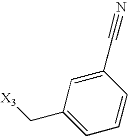 | 7.40 | 460.35 |
| 413 | 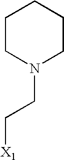 | 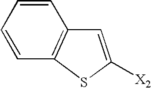 | 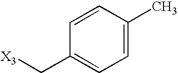 | 7.71 | 449.33 |
| 414 | 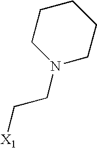 | 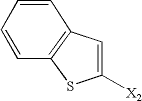 | 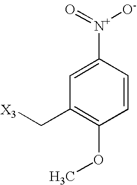 | 7.60 | 510.35 |
| 415 | 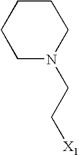 | 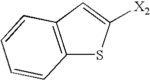 | 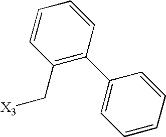 | 8.16 | 511.37 |
| 416 | 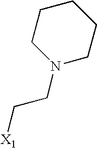 | 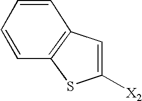 | 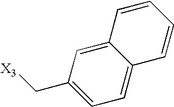 | 7.94 | 485.38 |
| 417 | 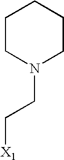 | 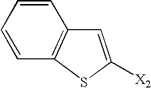 | 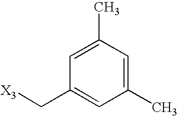 | 7.99 | 463.40 |
| 418 | 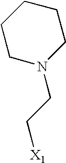 | 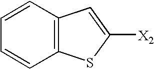 | 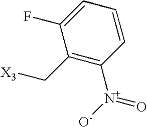 | 7.59 | 498.34 |
| 419 | 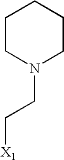 | 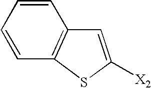 | 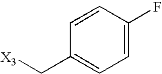 | 7.68 | 453.36 |
| 420 | 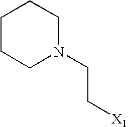 | 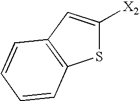 | 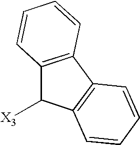 | 8.38 | 509.41 |
| 421 | 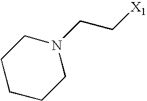 | 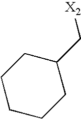 | 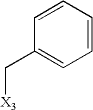 | 7.43 | 399.31 |
| 422 | 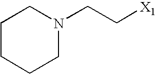 | 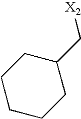 | 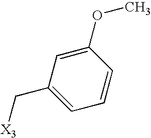 | 7.30 | 429.44 |
| 423 | 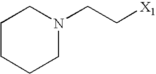 | 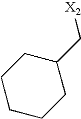 | 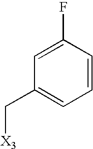 | 7.46 | 417.33 |
| 424 | 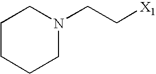 | 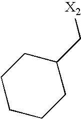 | 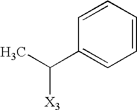 | 7.49 | 413.45 |
| 425 | 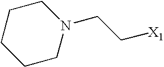 | 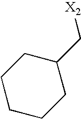 | 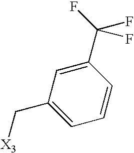 | 7.92 | 467.31 |
| 426 | 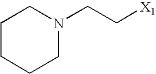 | 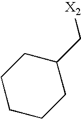 | 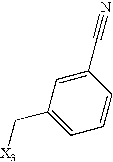 | 7.19 | 424.45 |
| 427 | 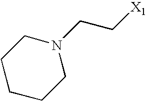 | 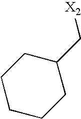 | 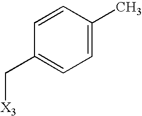 | 7.48 | 413.40 |
| 428 | 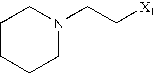 | 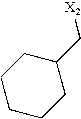 | 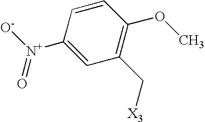 | 7.39 | 474.44 |
| 429 | 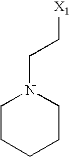 | 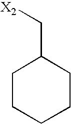 | 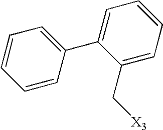 | 8.01 | 475.47 |
| 430 | 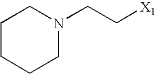 | 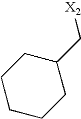 | 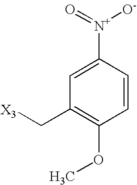 | 7.70 | 449.45 |
| 431 | 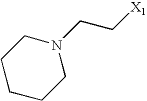 | 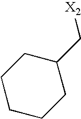 | 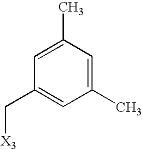 | 7.79 | 427.46 |
| 432 | 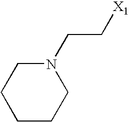 | 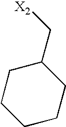 | 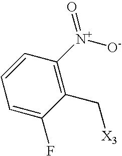 | 7.37 | 462.42 |
| 433 | 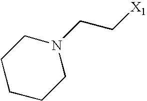 | 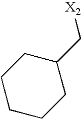 | 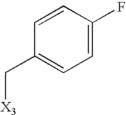 | 7.45 | 417.43 |
| 434 | 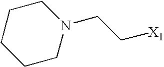 | 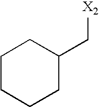 | 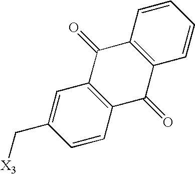 | 7.79 | 529.46 |
| 435 | 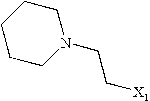 | 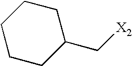 | 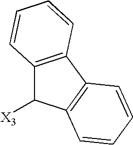 | 8.03 | 473.48 |
| 436 | 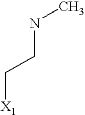 | 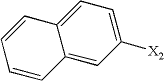 | 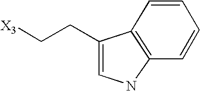 | 8.10 | 428.20 |
| 437 | 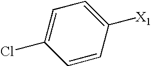 | 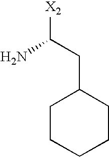 | 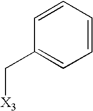 | 8.97 | 427.28 |
| 438 | 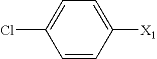 | 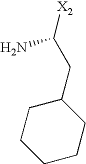 | 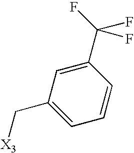 | 9.02 | 495.23 |
| 439 | 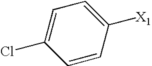 | 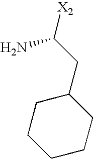 | 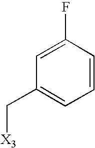 | 8.62 | 445.21 |
| 440 | 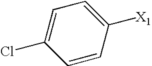 | 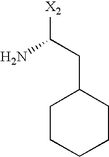 | 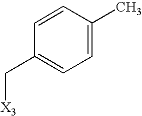 | 8.68 | 441.31 |
| 441 | 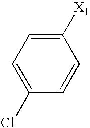 | 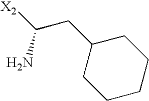 | 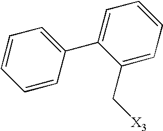 | 9.07 | 503.39 |
| 442 | 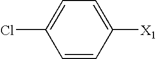 | 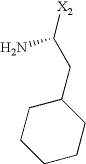 | 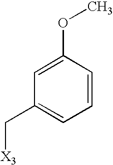 | 8.34 | 457.36 |
| 443 | 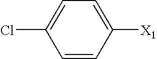 | 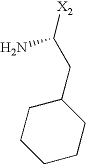 | 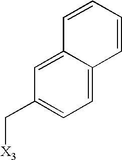 | 8.84 | 477.40 |
| 444 | 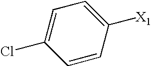 | 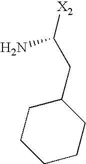 | 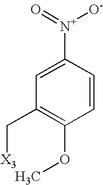 | 8.35 | 502.38 |
| 445 | 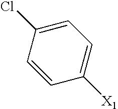 | 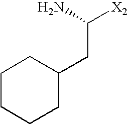 | 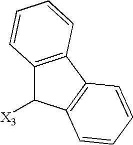 | 9.25 | 501.39 |
| 446 | 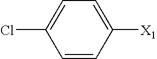 | 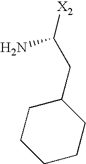 | 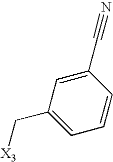 | 8.14 | 452.37 |
| 447 | 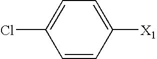 | 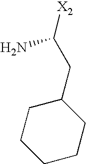 | 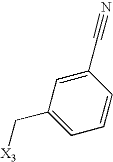 | 8.31 | 472.35 |
| 448 | 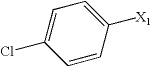 | 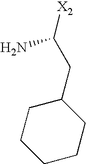 | 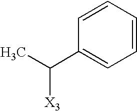 | 8.64 | 441.37 |
| 449 | 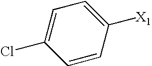 | 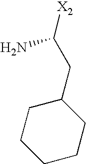 | 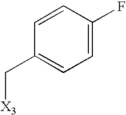 | 8.50 | 445.33 |
| 450 | 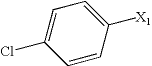 | 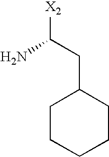 | 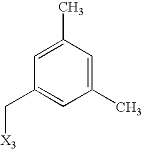 | 9.02 | 455.39 |
| 451 | 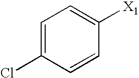 | 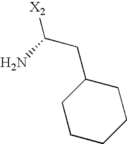 | 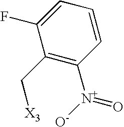 | 8.35 | 490.35 |
| 452 | 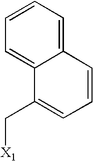 | 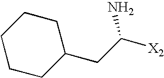 | 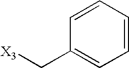 | 9.27 | 457.35 |
| 453 | 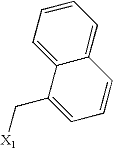 | 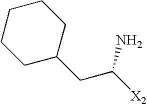 | 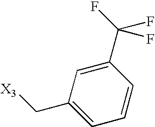 | 9.23 | 525.26 |
| 454 | 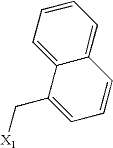 | 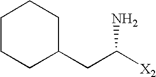 | 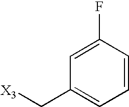 | 8.84 | 475.30 |
| 455 | 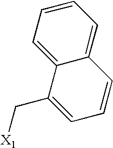 | 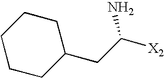 | 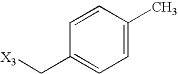 | 8.91 | 471.39 |
| 456 | 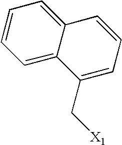 | 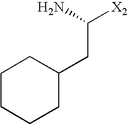 | 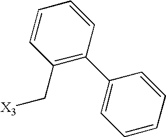 | 9.33 | 533.42 |
| 457 | 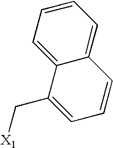 | 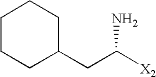 | 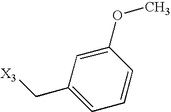 | 8.55 | 487.42 |
| 458 | 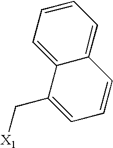 | 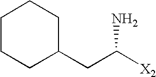 | 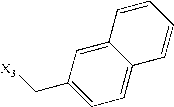 | 9.01 | 507.45 |
| 459 | 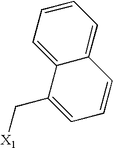 | 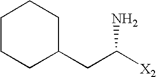 | 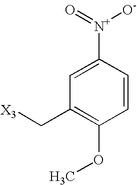 | 8.53 | 532.43 |
| 460 | 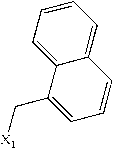 | 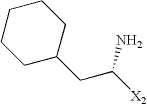 | 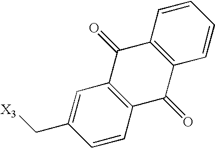 | 8.88 | 587.37 |
| 461 | 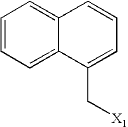 | 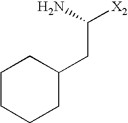 | 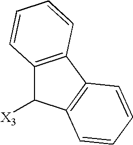 | 9.53 | 531.47 |
| 462 | 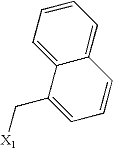 | 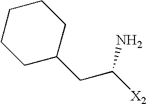 | 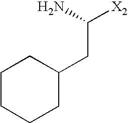 | 8.34 | 482.43 |
| 463 | 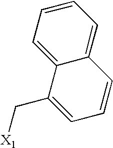 | 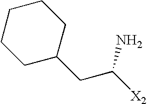 | 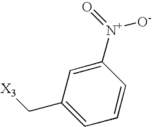 | 8.48 | 502.46 |
| 464 | 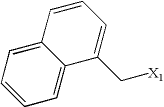 | 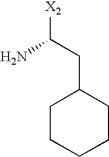 | 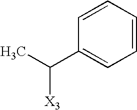 | 8.91 | 471.44 |
| 465 | 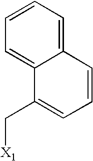 | 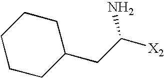 | 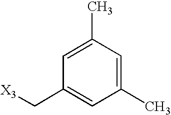 | 9.24 | 485.47 |
| 466 | 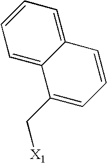 | 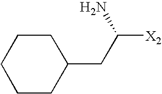 | 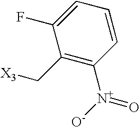 | 8.58 | 520.40 |
| 467 | 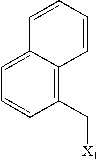 | 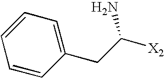 | 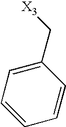 | 5.49 | 451.23 |
| 468 | 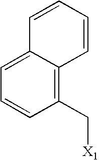 | 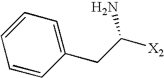 | 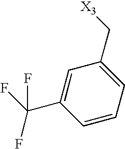 | 5.81 | 519.22 |
| 469 | 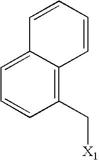 | 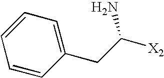 | 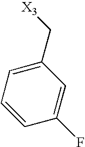 | 5.52 | 469.23 |
| 470 | 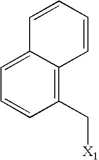 | 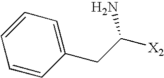 | 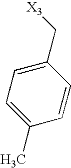 | 5.67 | 465.25 |
| 471 | 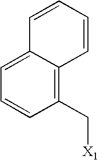 | 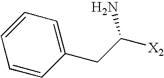 | 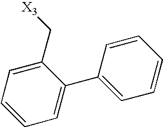 | 6.05 | 527.28 |
| 472 | 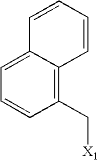 | 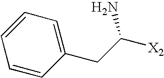 | 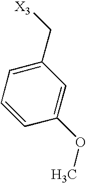 | 5.46 | 481.25 |
| 473 | 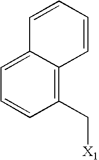 | 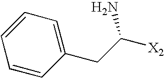 | 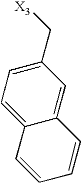 | 5.83 | 501.27 |
| 474 | 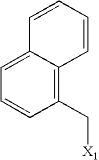 | 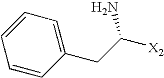 | 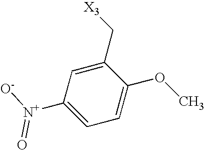 | 5.43 | 526.25 |
| 475 | 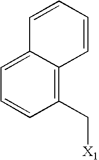 | 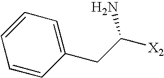 | 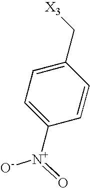 | 5.39 | 496.22 |
| 476 | 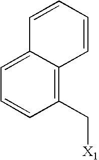 | 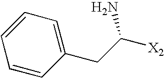 | 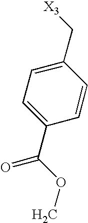 | 5.37 | 509.26 |
| 477 | 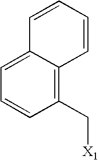 | 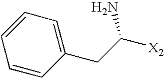 | 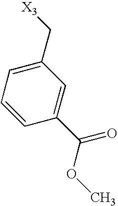 | 5.39 | 509.26 |
| 478 | 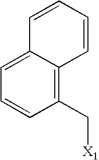 | 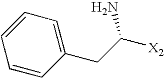 | 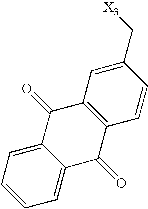 | 5.71 | 581.22 |
| 479 | 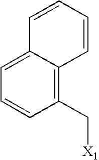 | 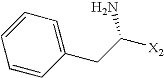 | 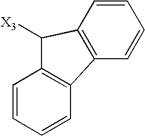 | 6.06 | 525.26 |
| 480 | 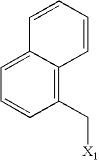 | 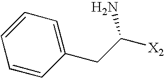 | 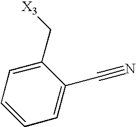 | 5.26 | 476.24 |
| 481 | 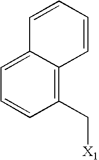 | 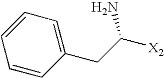 | 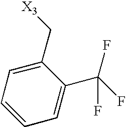 | 5.79 | 519.23 |
| 482 | 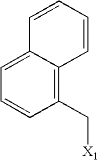 | 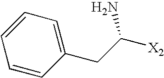 | 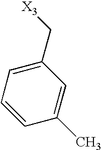 | 5.68 | 465.25 |
| 483 | 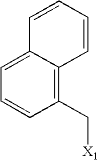 | 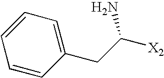 | 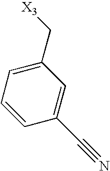 | 5.25 | 476.25 |
| 484 | 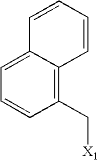 | 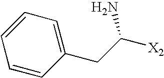 | 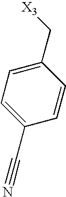 | 5.25 | 476.25 |
| 485 | 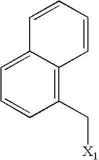 | 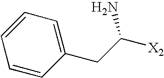 | 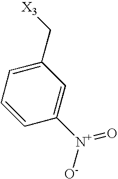 | 5.39 | 496.23 |
| 486 | 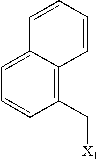 | 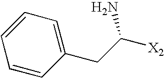 | 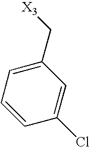 | 5.71 | 485.21 |
| 487 | 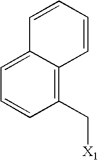 | 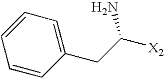 | 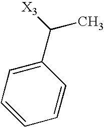 | 5.66 | 465.25 |
| 488 | 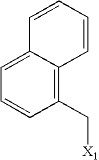 | 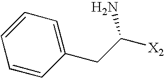 | 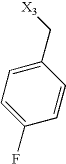 | 5.51 | 469.24 |
| 489 | 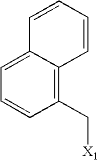 | 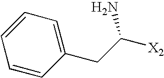 | 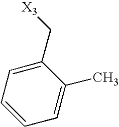 | 5.65 | 465.24 |
| 490 | 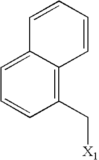 | 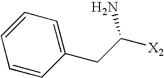 | 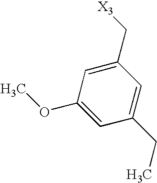 | 11.53 | 511.31 |
| 491 | 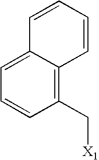 | 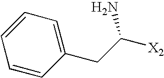 | 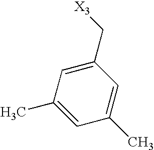 | 5.91 | 479.28 |
| 492 | 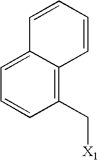 | 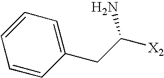 | 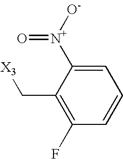 | 5.38 | 514.23 |
| 493 | 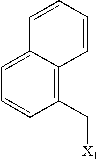 | 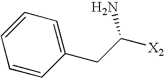 | 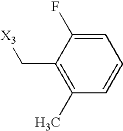 | 5.70 | 483.28 |
| 494 | 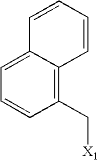 | 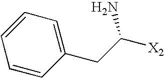 | 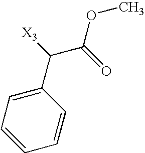 | 5.53 | 509.26 |
| 495 | 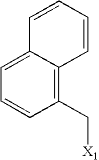 | 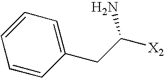 | 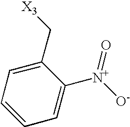 | 5.39 | 496.24 |
| 496 | 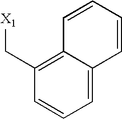 | 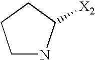 | 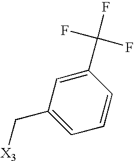 | 9.20 | 469.12 |
| 497 | 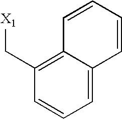 | 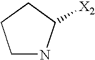 | 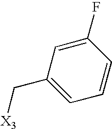 | 8.87 | 419.10 |
| 498 | 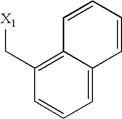 | 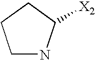 | 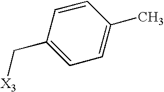 | 9.09 | 415.14 |
| 499 | 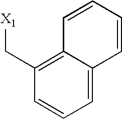 | 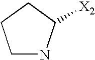 | 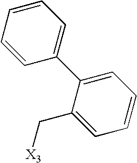 | 9.55 | 477.18 |

| | | | | | |
|---|---|---|---|---|---|
| 500 | 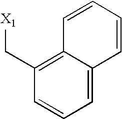 | 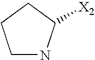 | 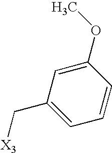 | 8.82 | 431.14 |
| 501 | 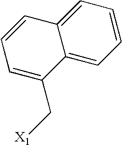 | 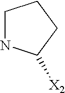 | 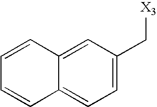 | 9.25 | 451.15 |
| 502 | 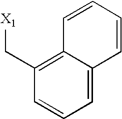 | 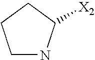 | 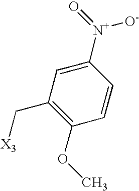 | 8.85 | 476.15 |
| 503 | 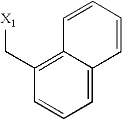 | 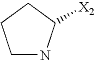 | 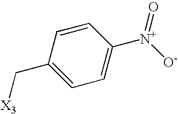 | 8.81 | 446.11 |
| 504 | 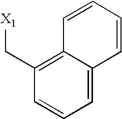 | 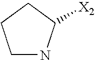 | 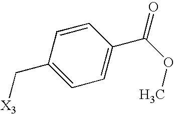 | 8.75 | 459.14 |
| 505 | 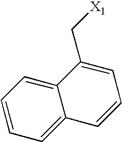 | 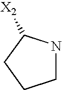 | 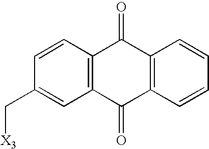 | 9.18 | 531.14 |
| 506 | 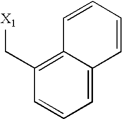 | 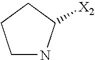 | 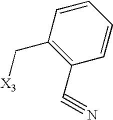 | 8.61 | 426.13 |
| 507 | 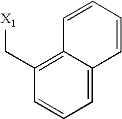 | 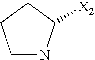 | 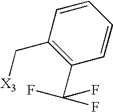 | 9.21 | 469.11 |
| 508 | 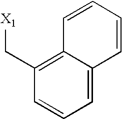 | 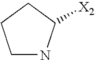 | 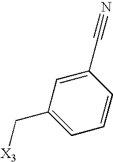 | 8.64 | 426.13 |
| 509 | 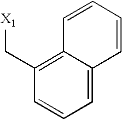 | 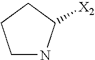 | 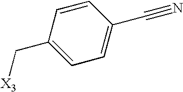 | 8.64 | 426.13 |
| 510 | 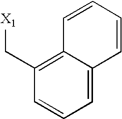 | 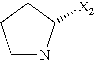 | 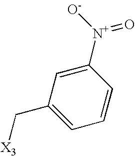 | 8.77 | 446.11 |
| 511 | 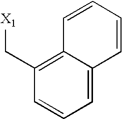 | 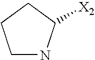 | 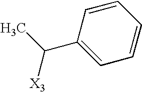 | 9.01 | 415.14 |
| 512 | 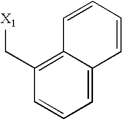 | 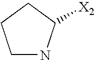 | 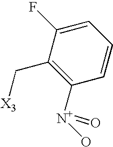 | 8.74 | 464.11 |
| 513 | 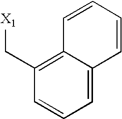 | 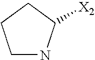 | 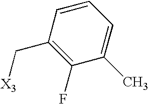 | 9.11 | 433.14 |
| 514 | 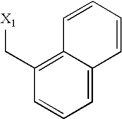 | 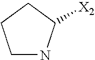 | 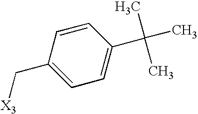 | 9.77 | 457.21 |
| 515 | 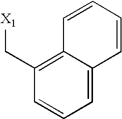 | 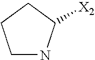 | 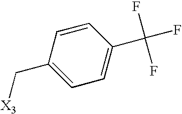 | 9.28 | 469.12 |
| 516 | 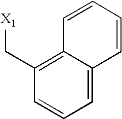 | 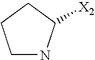 | 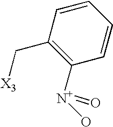 | 8.77 | 446.11 |
| 517 | 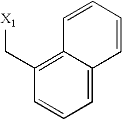 | 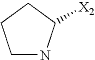 | 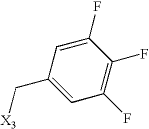 | 9.09 | 455.09 |
| 518 | 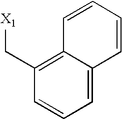 | 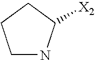 | 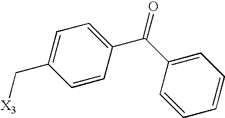 | 9.25 | 505.17 |
| 519 | 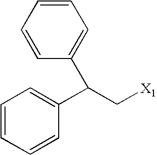 | 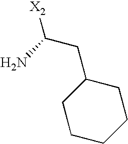 | 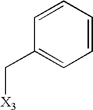 | 9.56 | 497.37 |
| 520 | 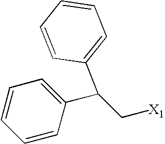 | 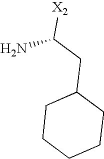 | 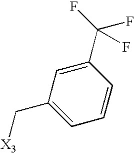 | 9.51 | 565.28 |
| 521 | 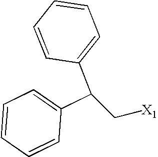 | 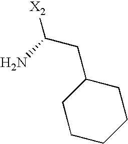 | 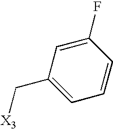 | 9.11 | 515.32 |
| 522 | 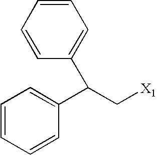 | 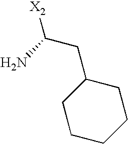 | 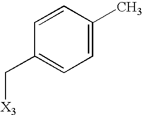 | 9.15 | 511.44 |
| 523 | 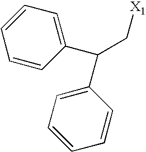 | 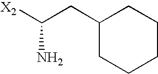 | 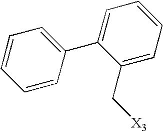 | 9.58 | 573.42 |
| 524 | 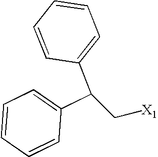 | 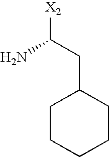 | 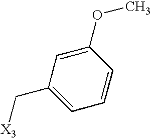 | 8.83 | 527.44 |
| 525 | 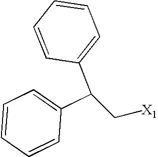 | 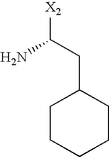 | 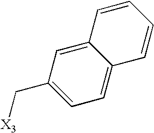 | 9.24 | 547.45 |
| 526 | 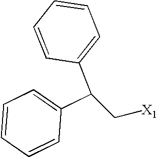 | 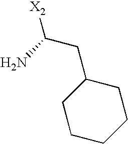 | 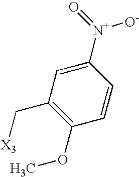 | 8.83 | 572.42 |
| 527 | 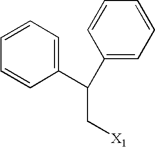 | 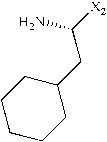 | 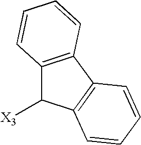 | 9.78 | 571.48 |
| 528 | 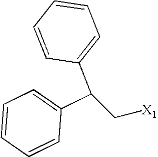 | 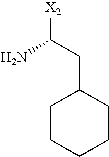 | 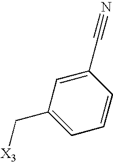 | 8.61 | 522.47 |
| 529 | 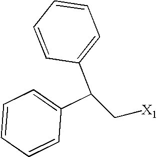 | 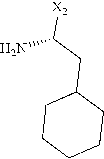 | 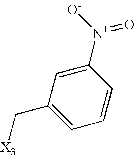 | 8.80 | 542.45 |
| 530 | 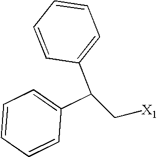 | 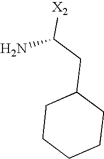 | 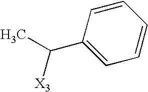 | 9.12 | 511.51 |
| 531 | 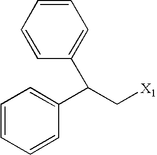 | 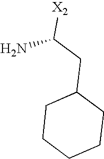 | 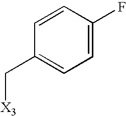 | 9.00 | 515.46 |
| 532 | 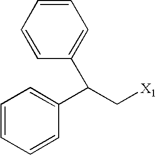 | 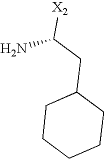 | 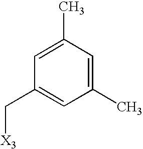 | 9.49 | 525.49 |
| 533 | 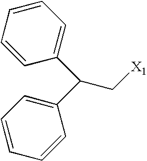 | 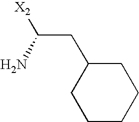 | 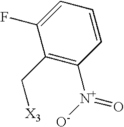 | 8.90 | 560.41 |
| 534 | 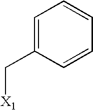 | 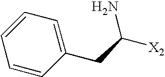 | 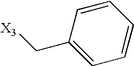 | 4.74 | 401.2 |
| 535 | 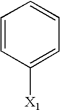 | 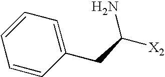 | 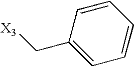 | 4.67 | 387.2 |
| 536 | 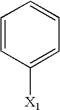 | 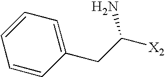 | 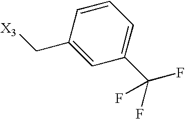 | 5.43 | 455.18 |
| 537 | 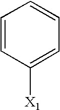 | 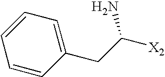 | 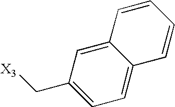 | 5.46 | 437.21 |
| 538 | 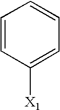 | 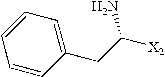 | 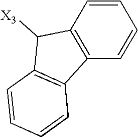 | 8.40 | 461.29 |
| 539 | 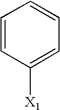 | 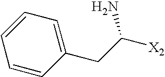 | 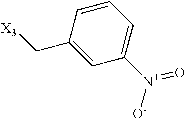 | 7.63 | 432.24 |
| 540 | 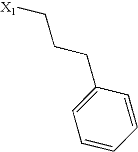 | 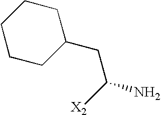 | 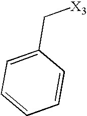 | 8.62 | 435.39 |
| 541 | 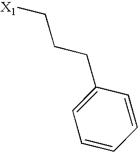 | 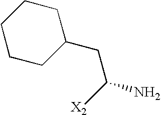 | 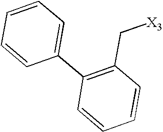 | 9.46 | 511.45 |
| 542 | 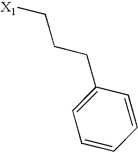 | 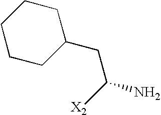 | 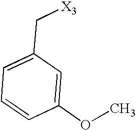 | 8.63 | 465.43 |
| 543 | 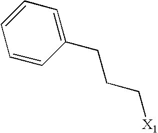 | 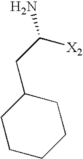 | 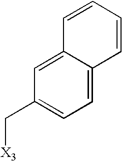 | 9.08 | 485.44 |
| 544 | 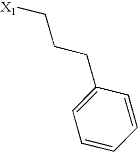 | 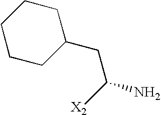 | 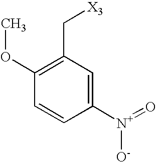 | 8.59 | 510.40 |
| 545 | 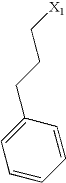 | 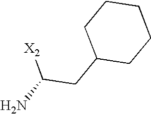 | 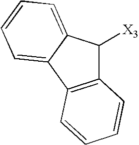 | 9.40 | 509.43 |
| 546 | 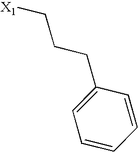 | 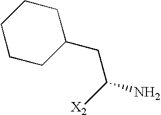 | 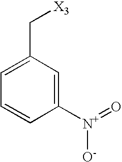 | 8.54 | 480.41 |
| 547 | 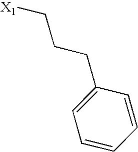 | 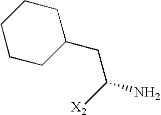 | 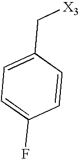 | 8.62 | 453.41 |
| 548 | 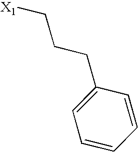 | 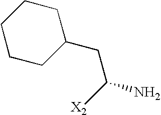 | 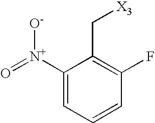 | 8.52 | 498.39 |
| 549 | 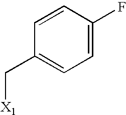 | 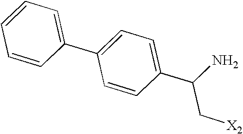 | 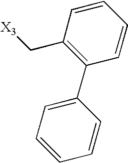 | 9.51 | 570.89 |
| 550 | 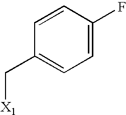 | 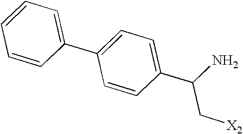 | 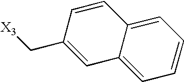 | 9.21 | 544.90 |
| 551 | 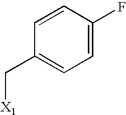 | 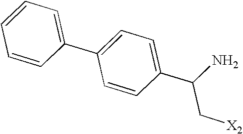 | 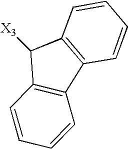 | 9.51 | 568.87 |
| 552 | 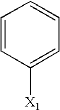 | 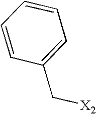 | 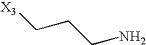 | 3.70 | 325.20 |
| 553 | 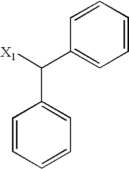 | 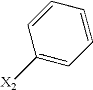 | 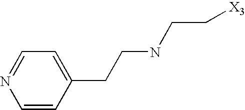 | 8.09 | 520.34 |
| 554 | 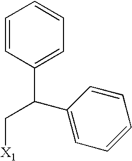 | 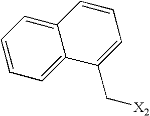 | 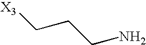 | 8.50 | 479.30 |
| 555 | 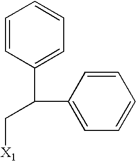 | 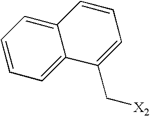 | 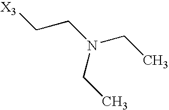 | 8.90 | 521.30 |
| 556 | 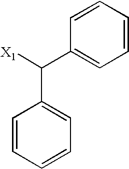 | 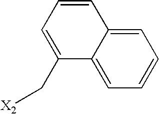 | 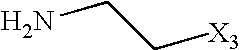 | 8.95 | 465.33 |
| 557 | 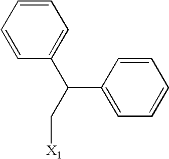 | 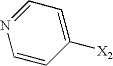 | 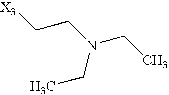 | 7.90 | 458.20 |
| 558 | 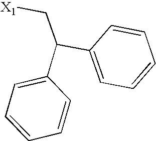 | 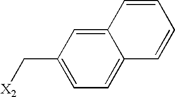 | 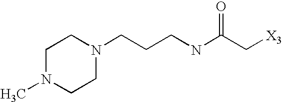 | 8.40 | 619.30 |
| 559 | 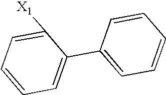 | 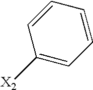 | 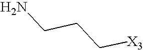 | 8.39 | 401.31 |
| 560 | 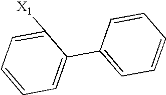 | 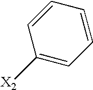 | 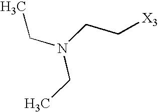 | 8.71 | 443.35 |
| 561 | 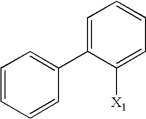 | 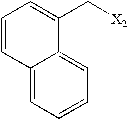 | 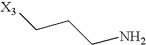 | 8.30 | 461.30 |
| 562 | 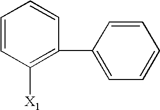 | 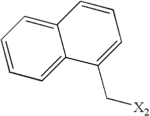 | 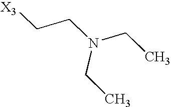 | 8.70 | 493.30 |
| 563 | 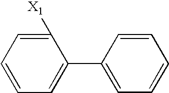 | 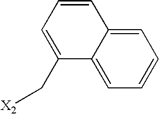 | 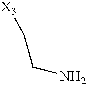 | 8.80 | 437.29 |
| 564 | 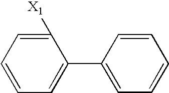 | 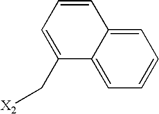 | 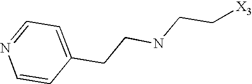 | 8.21 | 542.33 |
| 565 | 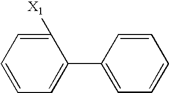 | 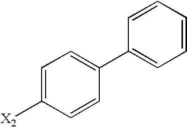 | 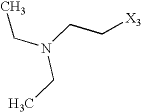 | 9.50 | 505.20 |
| 566 | 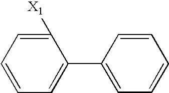 | 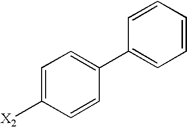 | 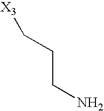 | 8.74 | 462.93 |
| 567 | 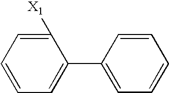 | 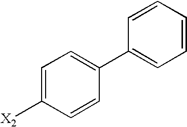 | 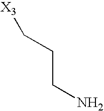 | 9.10 | 463.30 |
| 568 | 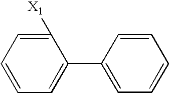 | 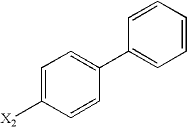 | 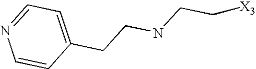 | 8.40 | 554.20 |
| 569 | 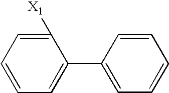 | 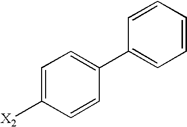 | 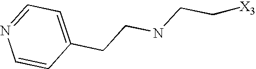 | 8.38 | 554.33 |
| 570 | 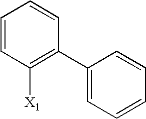 | 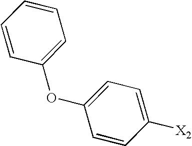 | 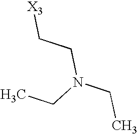 | 9.40 | 521.20 |
| 571 | 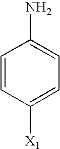 | 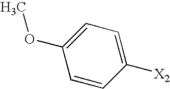 | 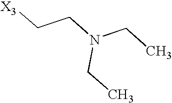 | 6.90 | 398.20 |
| 572 | 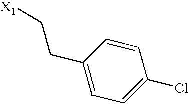 | 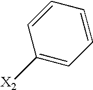 | 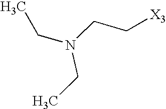 | 8.68 | 429.30 |
| 573 | 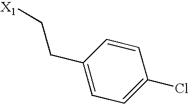 | 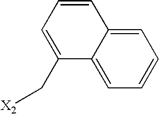 | 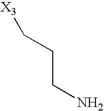 | 8.72 | 437.26 |
| 574 | 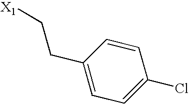 | 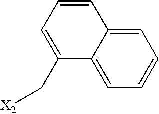 | 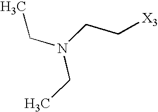 | 9.05 | 479.34 |
| 575 | 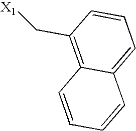 | 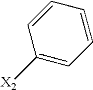 | 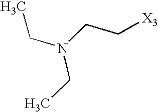 | 8.62 | 431.34 |
| 576 | 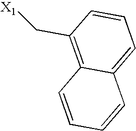 | 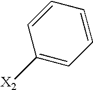 | 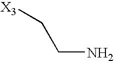 | 8.31 | 375.31 |
| 577 | 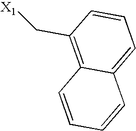 | 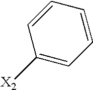 | 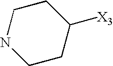 | 8.37 | 415.29 |
| 578 | 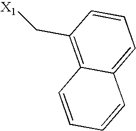 | 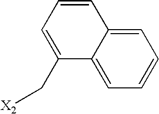 | 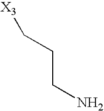 | 8.72 | 439.31 |
| 579 | 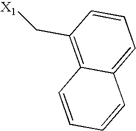 | 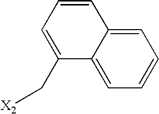 | 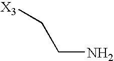 | 8.68 | 425.29 |
| 580 | 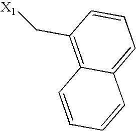 | 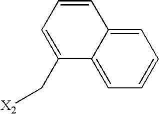 | 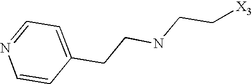 | 8.16 | 530.37 |
| 581 | 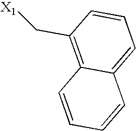 | 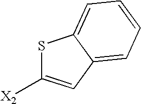 | 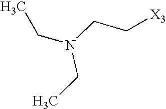 | 9.02 | 473.29 |
| 582 | 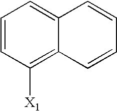 | 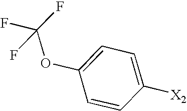 | 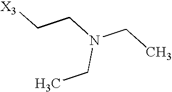 | 8.90 | 487.20 |
| 583 | 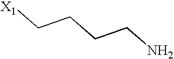 | 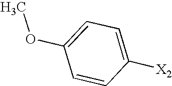 | 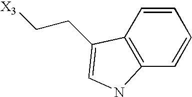 | 7.84 | 422.16 |
| 584 | 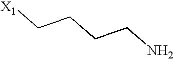 | 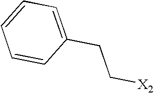 | 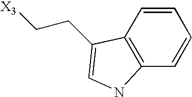 | 7.95 | 420.18 |
| 585 | 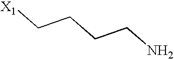 | 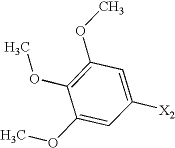 | 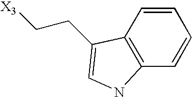 | 7.90 | 482.20 |
| 586 | 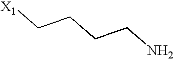 | 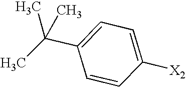 | 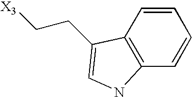 | 8.35 | 448.21 |
| 587 | 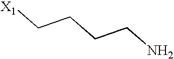 | 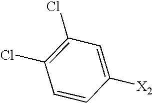 | 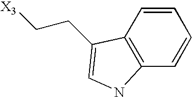 | 8.22 | 460.08 |
| 588 | 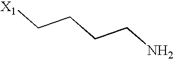 | 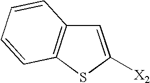 | 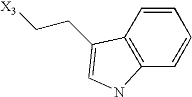 | 8.22 | 448.13 |
| 589 | 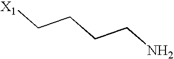 | 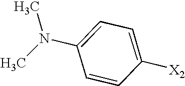 | 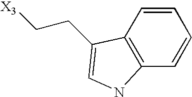 | 7.81 | 435.19 |
| 590 | 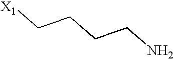 | 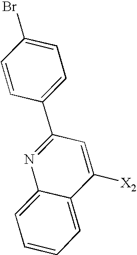 | 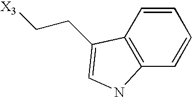 | 8.90 | 597.10 |
| 591 | 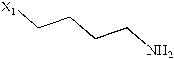 | 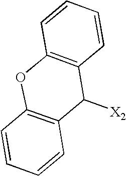 | 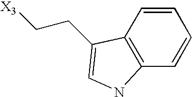 | 8.30 | 496.20 |
| 592 | 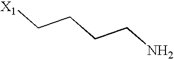 | 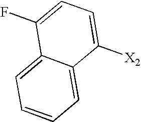 | 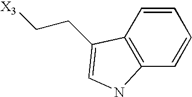 | 8.30 | 460.00 |
| 593 | 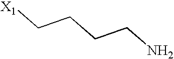 | 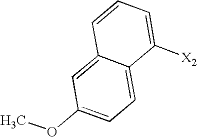 | 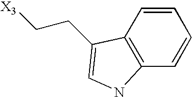 | 8.30 | 486.20 |
| 594 | 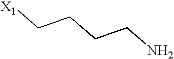 | 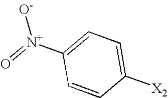 | 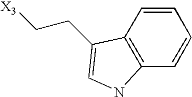 | 7.98 | 437.15 |
| 595 | 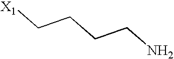 | 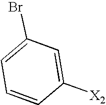 | 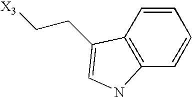 | 8.07 | 470.06 |
| 596 | 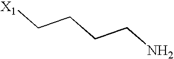 | 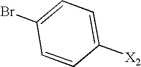 | 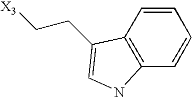 | 8.07 | 470.08 |
| 597 | 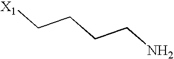 | 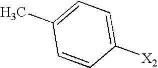 | 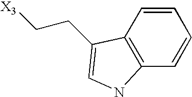 | 7.91 | 406.20 |
| 598 | 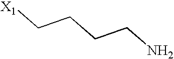 | 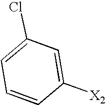 | 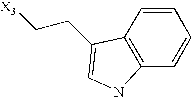 | 8.02 | 426.13 |
| 599 | 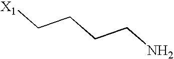 | 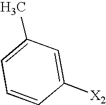 | 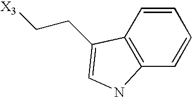 | 7.93 | 406.19 |
| 600 | 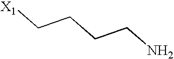 | 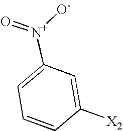 | 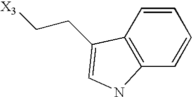 | 8.00 | 437.20 |
| 601 | 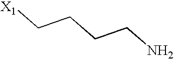 | 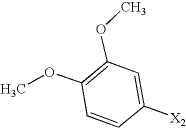 | 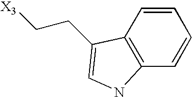 | 7.80 | 452.20 |
| 602 | 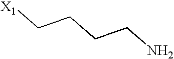 | 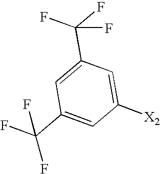 | 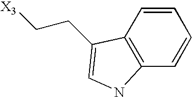 | 8.58 | 528.16 |
| 603 | 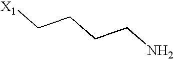 | 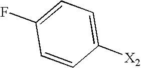 | 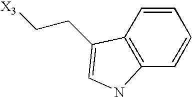 | 7.88 | 410.16 |
| 604 | 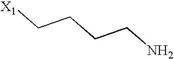 | 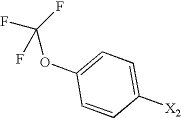 | 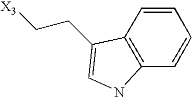 | 8.27 | 476.17 |
| 605 | 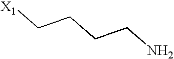 | 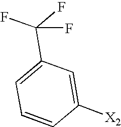 | 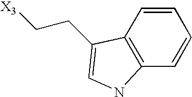 | 8.19 | 460.15 |
| 606 | 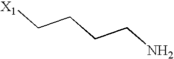 | 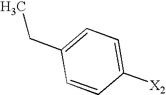 | 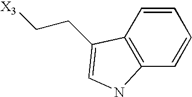 | 8.09 | 420.20 |
| 607 | 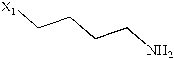 | 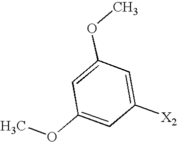 | 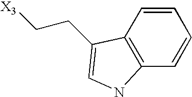 | 7.99 | 452.19 |
| 608 | 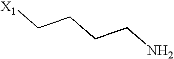 | 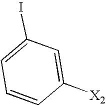 | 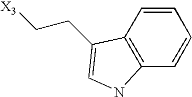 | 8.14 | 518.07 |
| 609 | 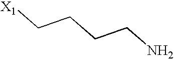 | 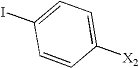 | 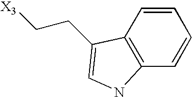 | 8.15 | 518.07 |
| 610 | 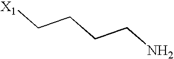 | 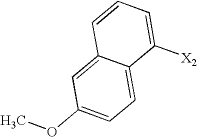 | 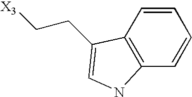 | 8.20 | 472.20 |
| 611 | 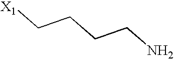 | 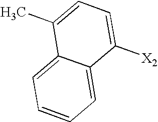 | 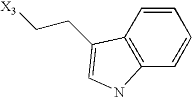 | 8.20 | 456.20 |
| 612 | 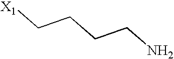 | 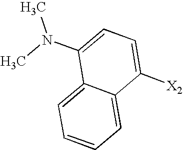 | 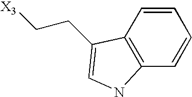 | 8.00 | 485.20 |
| 613 | 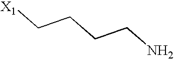 | 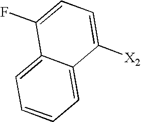 | 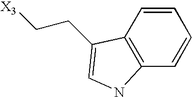 | 8.20 | 460.20 |
| 614 | 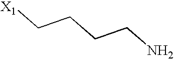 | 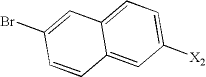 | 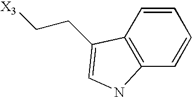 | 8.50 | 520.09 |
| 615 | 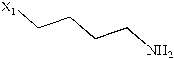 | 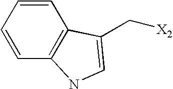 | 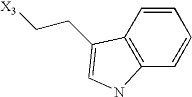 | 7.90 | 445.20 |
| 616 | 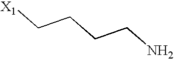 | 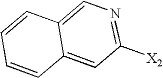 | 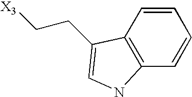 | 8.16 | 443.18 |
| 617 | 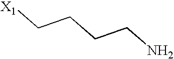 | 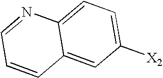 | 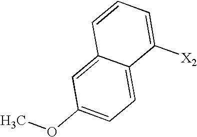 | 7.55 | 443.18 |
| 618 | 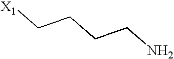 | 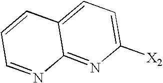 | 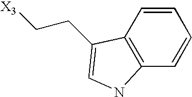 | 7.90 | 443.20 |
| 619 | 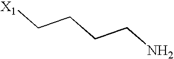 | 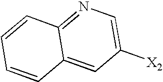 | 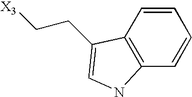 | 8.00 | 443.20 |
| 620 | 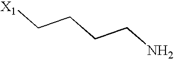 | 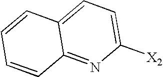 | 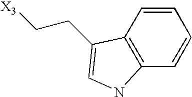 | 8.30 | 443.20 |
| 621 | 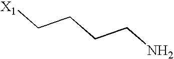 | 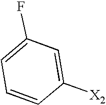 | 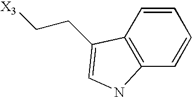 | 8.60 | 410.10 |
| 622 | 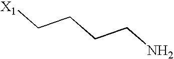 | 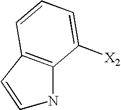 | 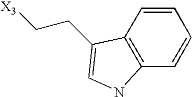 | 8.10 | 431.10 |
| 623 | 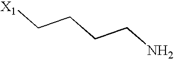 | 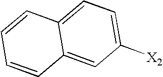 | 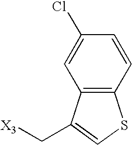 | 8.50 | 479.10 |
| 624 | 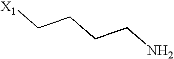 | 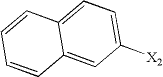 | 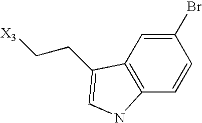 | 8.40 | 520.10 |
| 625 | 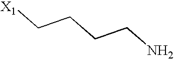 | 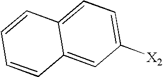 | 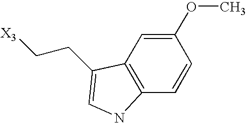 | 8.10 | 472.20 |
| 626 | 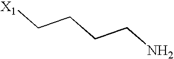 | 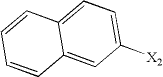 | 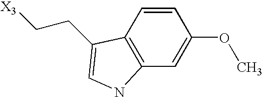 | 8.10 | 472.10 |
| 627 | 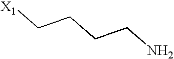 | 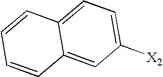 | 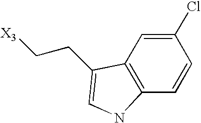 | 8.50 | 476.20 |
| 628 | 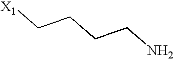 | 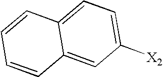 | 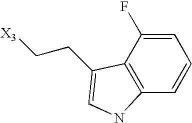 | 8.30 | 460.10 |
| 629 | 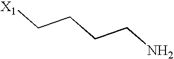 | 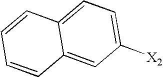 | 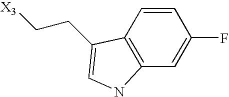 | 8.43 | 460.19 |
| 630 | 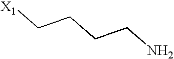 | 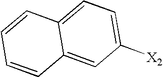 | 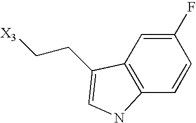 | 8.20 | 460.10 |
| 631 | 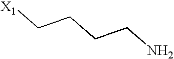 | 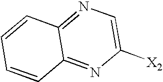 | 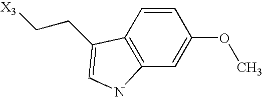 | 8.19 | 474.11 |
| 632 | 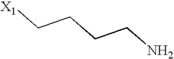 | 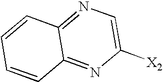 | 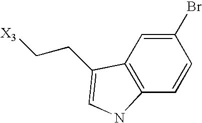 | 8.60 | 522.07 |
| 633 | 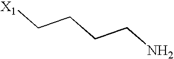 | 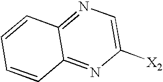 | 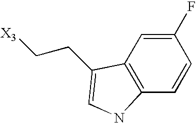 | 8.38 | 462.16 |
| 634 | 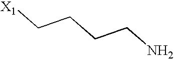 | 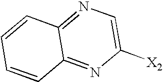 | 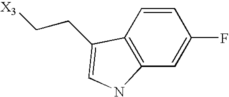 | 8.38 | 462.18 |
| 635 | 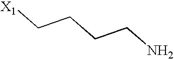 | 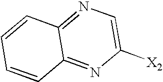 | 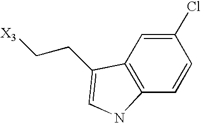 | 8.40 | 478.10 |
| 636 | 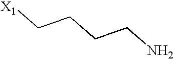 | 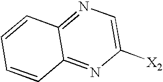 | 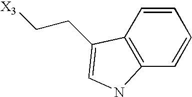 | 8.13 | 444.16 |
| 637 | 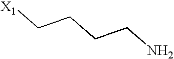 | 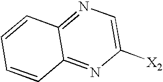 | 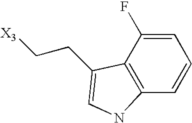 | 8.20 | 462.20 |
| 638 | 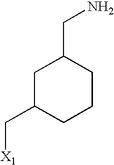 | 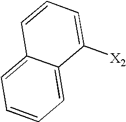 | 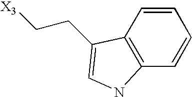 | 8.50 | 496.20 |
| 639 | 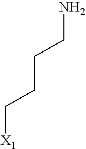 | 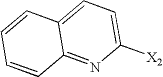 | 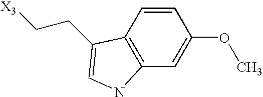 | 8.31 | 473.03 |
| 640 | 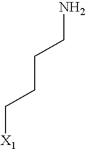 | 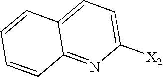 | 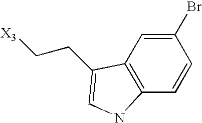 | 8.70 | 520.93 |
| 641 | 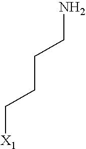 | 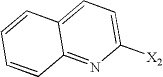 | 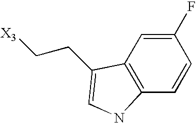 | 8.46 | 461.02 |
| 642 | 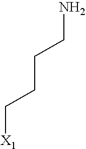 | 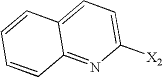 | 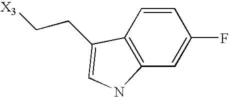 | 8.48 | 461.02 |
| 643 | 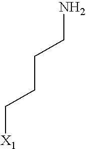 | 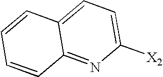 | 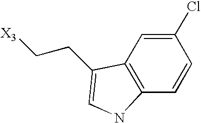 | 8.64 | 477.02 |
| 644 | 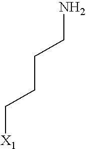 | 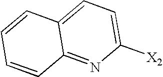 | 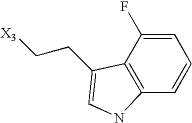 | 8.43 | 461.02 |
| 645 | 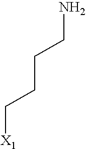 | 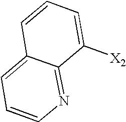 | 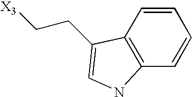 | 7.80 | 443.20 |
| 646 | 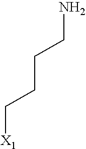 | 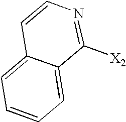 | 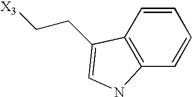 | 8.10 | 443.10 |
| 647 | 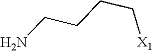 | 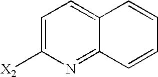 | 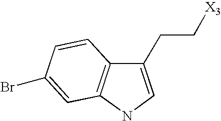 | 8.74 | 521.03 |
| 648 | 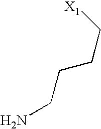 | 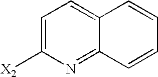 | 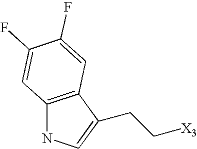 | 8.58 | 479.16 |
| 649 | 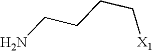 | 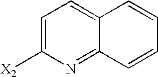 | 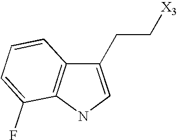 | 8.49 | 461.13 |
| 650 | 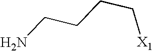 | 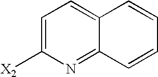 | 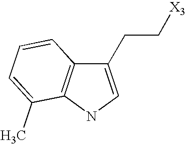 | 8.56 | 457.12 |
| 651 | 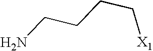 | 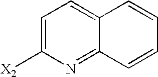 | 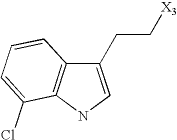 | 8.66 | 477.13 |
| 652 | 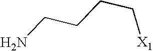 | 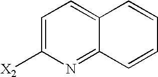 | 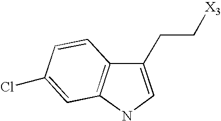 | 8.67 | 477.09 |
| 653 | 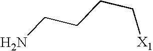 | 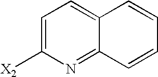 | 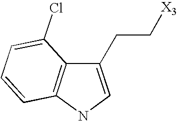 | 8.62 | 477.06 |
| 654 | 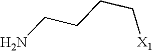 | 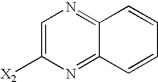 | 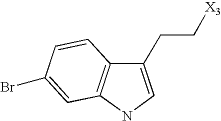 | 8.50 | 522.06 |
| 655 | 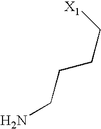 | 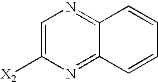 | 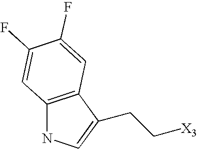 | 8.39 | 480.11 |
| 656 | 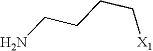 | 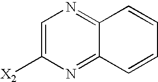 | 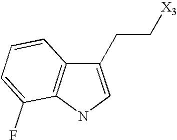 | 8.24 | 462.13 |
| 657 | 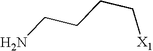 | 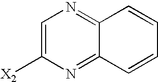 | 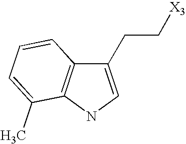 | 8.30 | 458.10 |
| 658 | 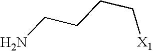 | 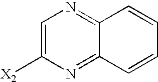 | 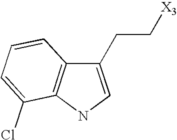 | 8.39 | 478.09 |
| 659 | 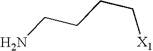 | 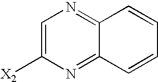 | 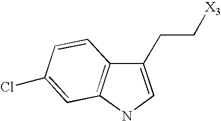 | 8.45 | 478.09 |
| 660 | 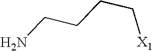 | 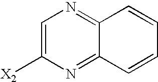 | 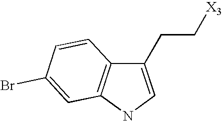 | 8.42 | 478.09 |
| 661 | 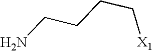 | 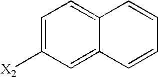 | 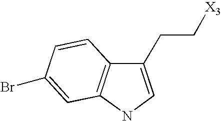 | 8.57 | 520.06 |
| 662 | 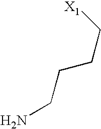 | 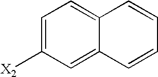 | 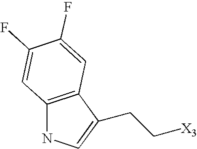 | 8.41 | 478.14 |
| 663 | 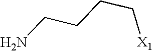 | 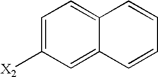 | 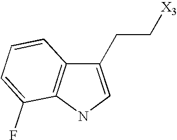 | 8.31 | 460.13 |
| 664 | 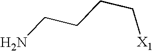 | 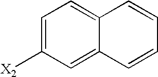 | 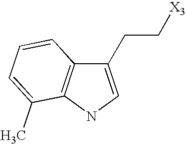 | 8.37 | 456.16 |
| 665 | 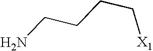 | 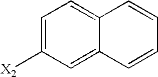 | 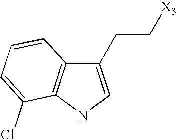 | 8.46 | 476.10 |
| 666 | 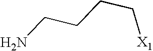 | 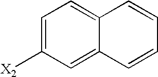 | 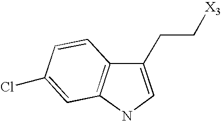 | 8.50 | 476.10 |
| 667 | 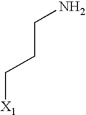 | 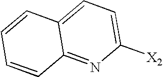 | 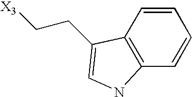 | 7.77 | 429.09 |
| 668 | 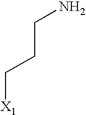 | 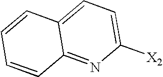 | 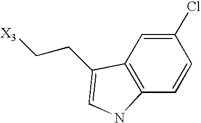 | 8.05 | 463.05 |
| 669 | 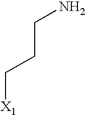 | 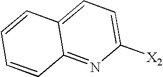 | 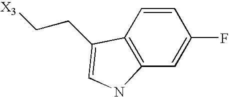 | 7.91 | 447.08 |
| 670 | 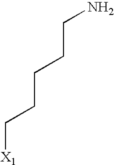 | 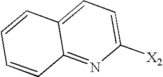 | 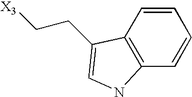 | 7.92 | 457.13 |
| 671 | 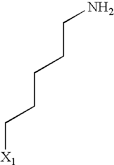 | 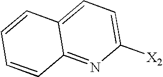 | 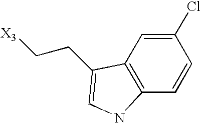 | 8.17 | 491.07 |
| 672 | 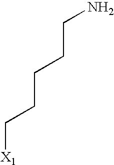 | 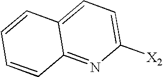 | 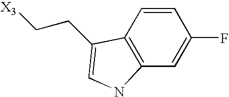 | 8.03 | 475.12 |
| 673 | 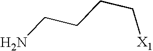 | 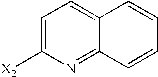 | 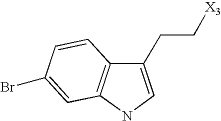 | 8.7 | 521.01 |
| 674 | 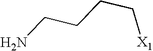 | 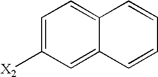 | 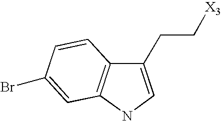 | 8.52 | 520.02 |
| 675 | 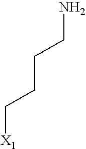 | 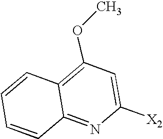 | 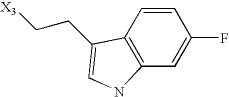 | 8.66 | 491.09 |
| 676 | 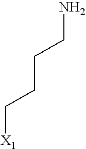 | 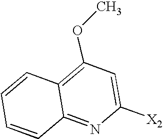 | 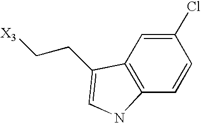 | 8.83 | 507.11 |
| 677 | 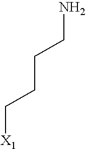 | 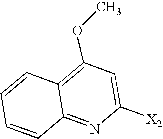 | 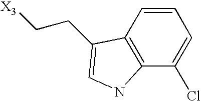 | 8.77 | 507.11 |
| 678 | 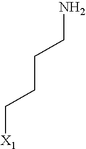 | 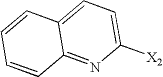 | 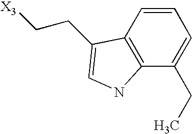 | 8.73 | 471.19 |
| 679 | 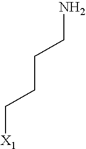 | 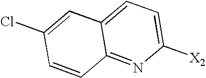 | 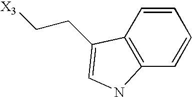 | 8.68 | 477.20 |
| 680 | 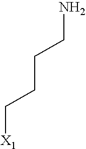 | 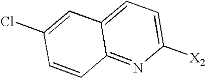 | 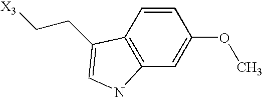 | 8.62 | 507.22 |
| 681 | 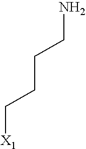 | 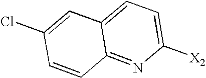 | 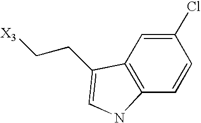 | 8.94 | 511.17 |
| 682 | 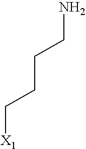 | 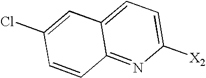 | 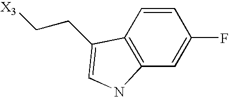 | 8.77 | 495.22 |
| 683 | 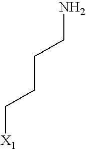 | 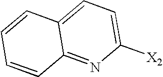 | 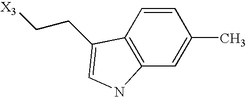 | 8.5 | 457.20 |
| 684 | 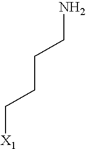 | 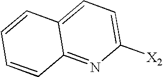 | 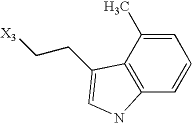 | 8.5 | 457.20 |
| 685 | 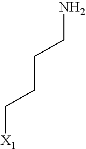 | 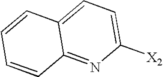 | 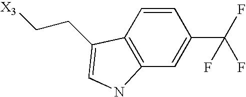 | 8.77 | 511.08 |
| 686 | 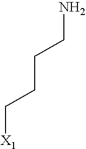 | 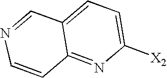 | 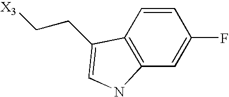 | 7.77 | 462.08 |
| 687 | 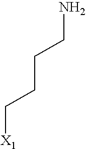 | 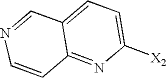 | 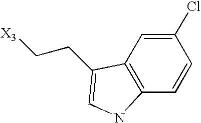 | 7.91 | 478.03 |
| 688 | 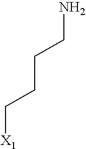 | 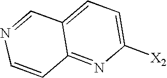 | 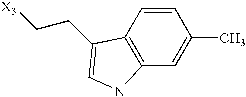 | 7.83 | 458.14 |
| 689 | 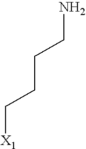 | 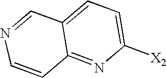 | 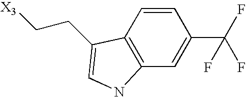 | 8.08 | 512.13 |
| 690 | 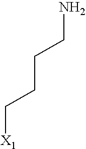 | 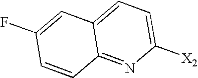 | 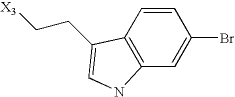 | 8.79 | 539.06 |
| 691 | 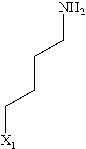 | 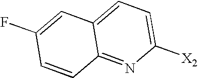 | 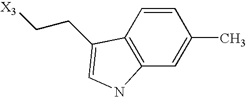 | 8.64 | 475.17 |
| 692 | 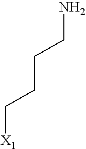 | 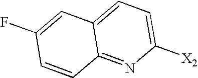 | 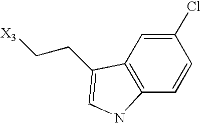 | 8.72 | 495.13 |
| 693 | 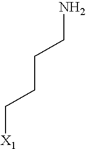 | 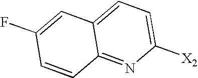 | 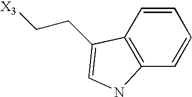 | 8.48 | 461.16 |
| 694 | 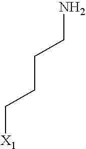 | 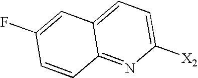 | 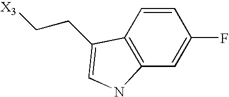 | 8.56 | 479.15 |
| 695 | 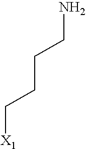 | 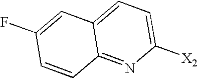 | 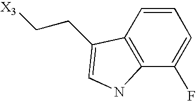 | 8.55 | 479.17 |
| 696 | 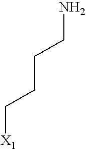 | 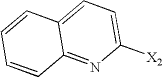 | 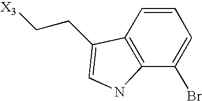 | 8.6 | 521.00 |
| 697 | 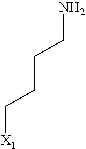 | 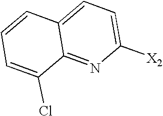 | 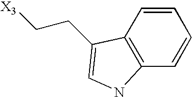 | 8.60 | 477.11 |
| 698 | 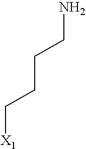 | 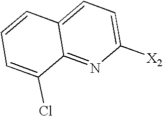 | 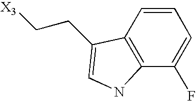 | 8.66 | 495.13 |
| 699 | 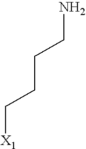 | 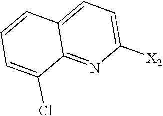 | 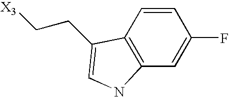 | 8.67 | 495.11 |
| 700 | 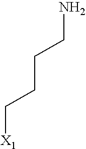 | 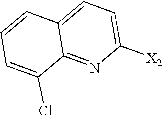 | 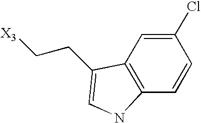 | 8.83 | 511.08 |
| 701 | 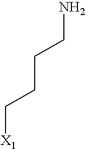 | 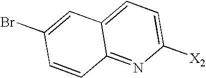 | 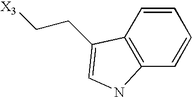 | 8.77 | 521.03 |
| 702 | 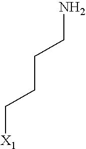 | 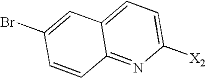 | 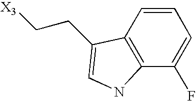 | 8.86 | 539.02 |
| 703 | 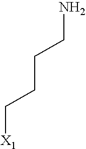 | 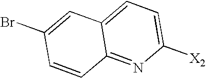 | 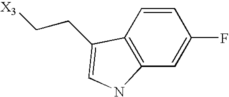 | 8.85 | 539.04 |
| 704 | 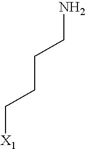 | 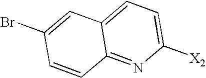 | 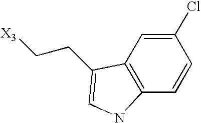 | 9.03 | 555.01 |
| 705 | 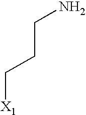 | 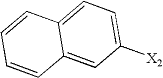 | 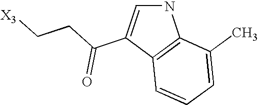 | 8.62 | 470.16 |
| 706 | 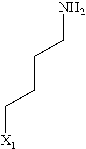 | 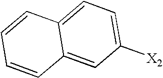 | 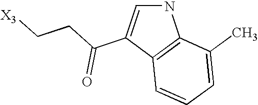 | 8.64 | 484.15 |
| 707 | 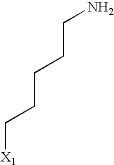 | 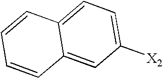 | 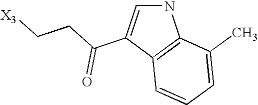 | 8.73 | 498.20 |
| 708 | 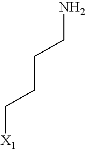 | 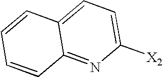 | 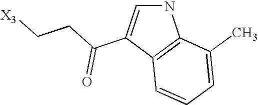 | 8.72 | 485.13 |

Pharmacological Study

The compounds of the present invention were tested as regards their affinity for different sub-types of somatostatin receptors according to the procedures described below.

Study of the Affinity for the Sub-Types of Human Somatostatin Receptors:

The affinity of a compound of the invention for the sub-type 2 receptor of somatostatin is determined by measurement of the inhibition of the bond of [¹²⁵I-Tyr¹¹]SRIF-14 to transfected CHO-K1 cells. The compounds showing an affinity are tested on the other sub-types, and optionally undergo a functional test as to their inhibition of the production of intracellular cAMP.

The gene of the sst₁ receptor of human somatostatin was cloned in the form of a genomic fragment. A segment PstI-XmnI of 1.5 Kb containing 100 bp of the non-transcribed 5′ region, 1.17 Kb of the coding region in totality, and 230 bp of the non-transcribed 3′ region is modified by the addition of the linker BglII. The resulting DNA fragment is subcloned in the BamHI site of a pCMV-81 in order to produce the expression plasmid in mammals (provided by Dr. Graeme Bell, Univ. Chicago). A cloned cell line expressing in a stable fashion the sst₁ receptor is obtained by transfection in CHO-KL cells (ATCC) using the calcium phosphate co-precipitation method. The plasmid pRSV-neo (ATCC) is included as selection marker. Cloned cell lines were selected in an RPMI 1640 medium containing 0.5 mg/ml of G418 (Gibco), followed by circular cloning and multiplication in culture.

The gene of the sst₂ receptor of human somatostatin, isolated in the form of a genomic fragment of DNA of 1.7 Kb BamHI-HindIII and subcloned in a plasmid vector pGEM3Z (Promega), was provided by Dr. G. Bell (Univ. of Chicago). The expression vector of the mammalian cells is constructed by inserting the BamH1-HindIII fragment of 1.7 Kb in endonuclease restriction sites compatible with the plasmid pCMV5. A cloned cell line is obtained by transfection in CHO-K1 cells using the calcium phosphate co-precipitation method. The plasmid pRSV-neo is included as selection marker.

The sst₃ receptor is isolated as a genomic fragment, and the complete coding sequence is contained in a BamHI/HindIII fragment of 2.4 Kb. The expression plasmid in mammals, pCMV-h3, is constructed by insertion of the NcoI-HindIII fragment of 2.0 Kb in the EcoR1 site of the vector pCMV after modification of the terminations and addition of EcoR1 linkers. A cloned cell line expressing in a stable fashion the sst₃ receptor is obtained by transfection in CHO-K1 cells (ATCC) by the calcium phosphate co-precipitation method. The plasmid pRSV-neo (ATCC) is included as selection marker. Cloned cell lines were selected in an RPMI 1640 medium containing 0.5 mg/ml of G418 (Gibco), followed by circular cloning and multiplication in culture.

The expression plasmid of the human sst₄ receptor, pCMV-HX, was provided by Dr. Graeme Bell (Univ. Chicago). This vector contains the genomic fragment coding for the human sst₄ receptor of 1.4 Kb NheI-NheI, 456 bp of the non transcribed 5′ region, and 200 bp of the non transcribed 3′ region, cloned in the XbaI/EcoR1 sites of PCMV-HX. A-cloned cell line expressing in a stable fashion the ss₄ receptor is obtained by transfection in CHO-K1 (ATCC) cells by the calcium phosphate co-precipitation method. The plasmid pRSV-neo (ATCC) is included as selection marker. Cloned cell lines were selected in an RPMI 1640 medium containing 0.5 mg/ml of G418 (Gibco), followed by circular cloning and multiplication in culture.

The gene corresponding to the human sst₅ receptor, obtained by the PCR method using a genomic λ clone as probe, was provided by Dr. Graeme Bell (Univ. Chicago). The resulting PCR fragment of 1.2 Kb contains 21 base pairs of the non-transcribed 5′ region, the coding region in totality, and 55 bp of the non-transcribed 3′ region. The clone is inserted in an EcoR1 site of the plasmid pBSSK(+). The insert is recovered in the form of a HindIII-XbaI fragment of 1.2 Kb for subcloning in an expression vector in mammals, pCVM5. A cloned cell line expressing in a stable fashion the sst₅ receptor is obtained by transfection in CHO-K1 cells (ATCC) by the calcium phosphate co-precipitation method. The plasmid pRSV-neo (ATCC) is included as selection marker. Cloned cell lines were selected in an RPMI 1640 medium containing 0.5 mg/ml of G418 (Gibco), followed by circular cloning and multiplication in culture.

The CHO-K1 cells which express in a stable fashion the human sst receptors are cultured in an RPMI 1640 medium containing 10% foetal calf serum and 0.4 mg/ml geneticin. The cells are collected with EDTA at 0.5 mM and centrifuged at 500 g for approximately 5 minutes at approximately 4° C. The centrifugate is resuspended in a Tris 50 mM buffer at pH 7.4 and centrifuged twice at 500 g for approximately 5 minutes at approximately 4° C. The cells are lysed by sonication then centrifuged at 39000 g for approximately 10 minutes at 4° C. The centrifugate is resuspended in the same buffer and centrifuged at 50000 g for approximately 10 minutes at approximately 4° C. and the membranes in the centrifugate obtained are stored at −80° C.

Competitive inhibition experiments of the bond of [¹²⁵I-Tyr¹¹]SRIF-14 are carried out in duplicate in 96-well polypropylene plates. The cell membranes are incubated with [¹²⁵I-Tyr¹¹]SRIF-14 for approximately 60 min. at approximately 37° C. in a HEPES 50 mM buffer (pH 7.4) containing BSA 0.2%, MgCl₂ 5 mM, Trasylol 200 KIU/ml, bacitricin 0.02 mg/ml and phenylmethylsulphonyl fluoride 0.02 mg/ml.

The bound [¹²⁵I-Tyr¹¹]SRIF-14 is separated from the free [¹²⁵I-Tyr¹¹]SRIF-14 by immediate filtration through GF/C glass fibre filter plates (Unifilter, Packard) pre-impregnated with 0.1% polyethylenimine (P.E.I.), using a Filtermate 196 (Packard). The filters are washed with 50 mM HEPES buffer at approximately 0-4° C. for approximately 4 seconds and their radioactivity is determined using a counter (Packard Top Count).

The specific bond is obtained by subtracting the non-specific bond (determined in the presence of 0.1 μM of SRIF-14) from the total bond. The data relative to the bond is analyzed to calculate the percentages of inhibition at a given concentration or to determine the inhibition constant values (Ki) according to the experiment.

Determination of the agonist or antagonist nature of a compound of the present invention is carried out using the test described below.

Functional Test: Inhibition of the Production of Intracellular cAMP:

CHO-K1 cells expressing the sub-types of human somatostatin receptors (SRIF-14) are cultured in 24-well plates in an RPMI 1640 medium with 10% foetal calf serum and 0.4 mg/ml geneticin. The medium is changed the day preceding the experiment.

The cells at a rate of 10⁵ cells/well are washed twice with 0.5 ml of new RPMI medium comprising 0.2% BSA completed by 0.5 mM of 3-isobutyl-1-methylxanthine (IBMX) and incubated for approximately 5 minutes at approximately 37° C.
the production of cyclic AMP is stimulated by the addition of 1 JM of forskolin (FSK) for 15-30 minutes at approximately 37° C.
the inhibitory effect of the somatostatin of an agonist compound is measured by the simultaneous addition of FSK (1 μM) and the compound to be tested (10⁻¹⁰ M to 10⁻⁵ M).
the antagonist effect of a compound is measured by the simultaneous addition of FSK (1 μM), SRIF-14 (1 nM) and of the compound to be tested (10⁻¹⁰ M to 10⁻⁵ M).

The reaction medium is eliminated and 200 μl of 0.1 N HCl is added. The quantity of cAMP is measured by a radioimmunological test (FlashPlate SMP001A kit, New England Nuclear).

Results:

The tests carried out according to the protocols described above made it possible to show that the products of general formula (I) defined in the present Application have a good affinity for at least one of the sub-types of somatostatin receptors, the inhibition constant Ki being lower than micromolar for certain exemplified compounds.

1. A compound of the formula 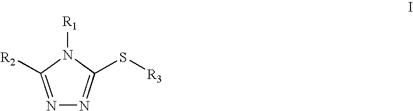
in racemic, enantiomeric form or all combination of these forms, wherein
one of R₁, R₂ or R₃ is (CH₂)ₙ—[Q]ₚ—(CH₂)ₘ—NXY or —(CH₂)ₙ—W,
W is a heterocycloalkyl containing at least one nitrogen atom;
Q is selected from the group consisting of —O—, —S—, —C(O)—NH—, —C(Z_{q})(Z_{q}′)—, aryl and (C₃-C₇)cycloalkyl;
Z_{q} and Z_{q}′ are individually selected from the group consisting of hydrogen, aryl optionally substituted by aryl, (C₃-C₇)cycloalkyl-alkyl, arylalkyl, —C(O)O—R and —C(O)—NH—R′;
R represents a is selected from the group consisting of (C₁-C₆)alkyl, aryl and aralkyl aryl and aralkyl being optionally substituted by at least one substituent selected from the group consisting of (C₁-C₆)alkoxy, hydroxy, halo, nitro cyano, amino, (C₁-C₆)alkylamino and di((C₁C₆)alkyl)amino;
R′ is selected from the group consisting of (C₁-C₆)alkyl, aryl, aralkyl, heteroaryl and heteroaryl-alkyl, the aryl, aralkyl, heteroaryl and heteroaryl-alkyl radicals being optionally substituted by at least one substituent selected from the group consisting of (C₁-C₆)alkoxy, hydroxy, halo, nitro cyano, amino, (C₁-C₆)alkylamino, and di((C₁-C₆)alkyl)amino;
X and Y are individually hydrogen, (C₁-C₆)alkyl, (C₁-C₆)alkoxy-carbonyl and heteroaryl-alkyl, or X and Y form together with the nitrogen atom on which they are attached, a heterocycloalkyl optionally substituted by a (C₁-C₆)alkyl;
p is 0 or 1; n and m are individually an integer from 0 to 6;
and the two other radicals represent Rs are, independently, —(CH₂)_{n′}[Q′]_{p′}[C(X′)(Y′)_{m′}Z′,
Q represents is selected from the group consisting of —O—, —S—, —C(O)—, —NH—, —CH═CH— or —C≡C—;
X′, Y′ and Z′ are, independently, selected from the group consisting of hydrogen, (C₁-C₆)alkyl, (C₁-C₆)alkoxy, (C₁-C₆)alkoxy-carbonyl, cyano, amino, (C₁C₆)alkylamino, dii((C₁-C₆)alkyl)amino, (C₃-C₇)cycloalkyl, heterocycloalkyl, aryl, heteroaryl, 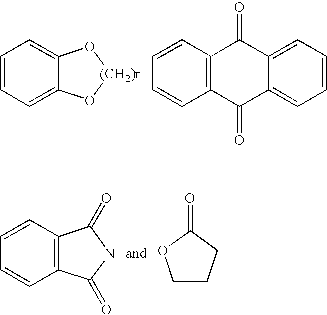
r is 1, 2 or 3
the (C₃-C₇)cycloalkyl, heterocycloalkyl, aryl and heteroaryl being optionally substituted by at least one substituent selected from the group consisting of —(CH₂)q′—X″—Y″, hydroxy, halo, nitro, cyano, amino, (C₁-C₆)alkylamino and di((C₁-C₆)alkyl)amino;
X″ represents is selected from the group consisting of —O—, —S—, —C(O)—, —C(O)—O—, —SO₂— and a covalent bond;
Y″ is (C₁-C₆)alkyl optionally substituted by at least one identical or different halo; or aryl or heteroaryl radical optionally substituted by at least one substituent selected from the group consisting of (C₁-C₆)alkoxy, hydroxy, halo, nitro, cyano, amino, (C₁-C₆)alkylamino and di((C I—C₆)alkyl)amino;
p′ is 0 or 1, and n′, m′ and q′ are, independently, an integer from 0 to 6;
excluding the compounds wherein
i) R₁ represents is (CH₂)₂—W and W representing is morpholino or piperazinyl, R₂ phenyl, m-chlorophenyl or 4-pyridyl, and R₃ is hydrogen;
ii) R₁ represents is (CH₂)₂—W and W is pyrrolidinyl, R₂ is p-chlorophenyl and R₃ is the hydrogen atom;
and their addition salts with pharmaceutically acceptable acids.

2. A compound of claim 1, wherein
R₁ is —(CH₂)ₙ—[Q]ₚ—(CH₂)ₘ—NXY;
Q represent is aryl or (C₃-C₇)cycloalkyl;
X and Y are independently selected from the group consisting of hydrogen, and (C₁-C₆)alkyl, or X and Y form together with the nitrogen atom on which they are attached, a heterocycloalkyl optionally substituted by (C₁-C₆)alkyl;
p 0 or 1, and n and m are, independently, an integer from 0 to 6;
R₂ is —(CH₂)_{n′}[Q′]_{p′}[C(X′)(Y′)_{m′}Z′;
Q′ is —O—,
X′ is hydrogen;
Y′ and Z′, are independently selected from the group consisting of hydrogen, (C₁-C₆)alkyl, cyano, amino, (C₃-C₇)cycloalkyl, aryl and heteroaryl;
the aryl and heteroaryl being optionally substituted by at least one substituent selected from the group consisting of —(CH₂)_{q}—X″—Y″, hydroxy, halo, nitro, amino, (C₁-C₆)alkylamino and di((C₇-C₆)alkyl)amino;
X″ is selected from the group consisting of —O—, —S— and a covalent bond;
Y″ represents is (C₁-C₆)alkyl radical optionally substituted by at least one identical or different halo, or aryl optionally substituted by at least one identical or different halo;
p is 0 or 1; n′ is 0, 1 or 2; and m′ is an integer from 1 to 6;
R₃ is —(CH₂)_{n′}[Q′]_{p′}[C(X′)(Y′)_{m′}Z′;
Q′ is selected from the group consisting of —O—, —C(O)—, —CH═CH— and —C≡C—;
X′ is hydrogen;
Y′ and Z′ represent are independently selected from the group consisting of hydrogen, (C₁-C₆)alkyl, (C₁-C₆)alkoxy-carbonyl, (C₃-C₇)cycloalkyl, aryl, heteroaryl,
the aryl and heteroaryl being optionally substituted by at least one substituent selected from the group consisting of —(CH₂)_{q}′—X″—Y″, halo, nitro, cyano, and di((C₁-C₆)alkyl)amino;
X″ is selected from the group consisting of —O—, —C(O)—, —C(O)—O—, —SO₂— and a covalent bond;
Y″ is (C₁-C₆)alkyl optionally substituted by at least one identical or different halo, or an alkyl;
p′ represents is 0 or 1; n′ and m′ are an integer from 0 to 6.

3. A compound of claim 2, wherein
the aryl of Q is phenyl; the (C₃-C₇)cycloalkyl of Q is the cyclohexyl;
the heterocycloalkyl formed by X and Y together with the nitrogen atom on which they are attached, is selected from the group consisting of pyrrolidine, piperidine, piperazine and morpholine;
the (C₃-C₇)cycloalkyl, independently of Y′ and Z′, is cyclohexyl;
the aryl, independently of Y′ and Z′, is selected from the group consisting of phenyl, naphthyl and fluorenyl;
the heteroaryl, independently of Y′ and Z′ of R₂ is selected from the group consisting of thienyl, furyl, benzothienyl, pyridyl, indolyl, thiadiazolyl, quinolyl, isoquinolyl, quinoxalyl, xanthenyl and naphthyridyl;
the heteroaryl, independently of Y′ and Z′ of R₃ is selected from the group consisting of benzothienyl, furyl, indolyl and isoxazolyl; and
the aryl of Y″ is phenyl radical.

4. A compound of claim 1, wherein
R₁ is —(CH₂)_{n′}[Q′]_{p′}C(X′)(Y′)_{m′}Z′;
X′ is hydrogen;
Y′ and Z′ are independently selected from the group consisting of hydrogen, (C₁-C₆)alkyl, and aryl optionally substituted by at least one substituent selected from the group consisting of —(CH₂)_{q′}—X″Y″, halo, and amino;
X″ is a covalent bond;
Y″ is aryl;
p′ is 0, n′ is 0 or 1, and m′ is an integer from 0 to 6;
R₂ is —(CH₂)ₙ—[Q]ₚ—(CH₂)ₘ—NXY or —(CH₂)ₙ—W;
W is heterocycloalkyl containing at least one nitrogen atom;
Q is —C(Z_{q})(Z_{q}′)—;
Z_{q} is hydrogen;
Z_{q}′ is selected from the group consisting of hydrogen, aryl optionally substituted by aryl, (C₃-C₇)cycloalkyl-alkyl and aralkyl;
X and Y are independently selected from the group consisting of hydrogen, (C₁-C₆)alkyl and (C₁-C₆)alkoxy-carbonyl;
p is 0 or 1, and n is 0 or 1, and m is an integer from 0 to 6;
R₃ is —(CH₂)_{n′}[Q′]_{p′}[C(X′)(Y′)]_{m′}Z′;
Q is selected from the group consisting of —O—, —C(O)—, —CH═CH— and —C≡C—;
X′ is hydrogen;
Y′ and Z′ are independently selected from the group consisting of hydrogen, (C₁-C₆)alkyl, (C₁-C₆)alkoxy-carbonyl, (C₃-C₇)cycloalkyl, aryl, heteroaryl, 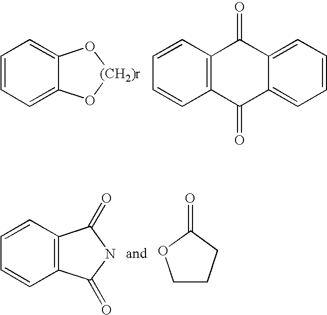
r is 1, 2 or 3
the aryl and heteroaryl being optionally substituted by at least one substituent selected from the group consisting of —(CH₂)_{q}—X″—Y″, halo, nitro, cyano, and di((C₁-C₆)alkyl)amino;
X″ is selected from the group consisting of —O—, —C(O)—, —C(O)—O—, —SO₂— and a covalent bond;
Y″ is (C₁-C₆)alkyl optionally substituted by at least one identical or different halo and aryl;
r is 1, 2 or 3; p′ is 0 or 1; n′ and m′ are an integer from 0 to 6.

5. A compound of claim 4, wherein
the aryl, independently of Y′ and Z′ or R₁ is phenyl or naphthyl;
the heterocycloalkyl of W, is piperidine or pyrrolidine ring;
the aryl of Z_{q}′, is phenyl or naphthyl;
the aryl of Z_{q}′, is phenyl;
the aralkyl of Z_{q}′, is benzyl;
the (C₃-C₇)cycloalkyl of the —(C₃-C₇)cycloalkyl-alkyl of Z_{q}′, is cyclohexyl;
the (C₃-C₇)cycloalkyl, independently of Y′ and Z′, is cyclohexyl radical,
the aryl, independently of Y′ and Z′ of R₃ is selected from the group consisting of phenyl, naphthyl and fluorenyl;
the heteroaryl, independently of Y′ and Z′ of R₃ is selected from the group consisting of benzothienyl, furyl, indolyl and isoxazolyl; and
the aryl of Y″ is phenyl.

6. A compound of claim 1, wherein
R₁ represents a radical of formula —(CH₂)_{n′}[Q′]_{p′}[C(X′)(Y′)]_{m′}Z′;
X′ is hydrogen;
Y′ and Z′ are independently selected from the group consisting of hydrogen, (C₁-C₆)alkyl, and aryl optionally substituted by at least one substituent selected from the group consisting of —(CH₂)_{q′}—X″—Y″, halo and amino;
X″ is a covalent bond;
Y″ is aryl;
p′ is 0, n′ is 0 or 1, and m′ is an integer from 0 to 6;
R₂ is —(CH₂)_{n′}[Q′]_{p′}[C(X′)(Y′)]_{m′}Z′;
Q′ is —O—;
X′ is hydrogen;
Y′ and Z′ are independently selected from the group consisting of hydrogen, (C₁-C₆)alkyl, cyano, amino, (C₃-C₇)cycloalkyl, aryl and heteroaryl;
the aryl and heteroaryl being optionally substituted by at least one substituent selected from the group consisting of —(CH₂)_{q}—X″—Y′, hydroxy, halo, nitro, amino, (C₁-C₆)alkylamino and di((C₁-C₆)alkyl)amino;
X″ is —O—, —S— and a covalent bond;
Y″ is (C₁-C₆)alkyl optionally substituted by at least one identical or different halo, or aryl optionally substituted by at least one or more identical or different halo radicals;
p′ is 0 or 1; n′ 0, 1 or 2; and m′ is an integer from 0 to 6;
R₃ represents a radical of formula is —(CH₂)ₙ—[Q]ₚ—(CH₂)ₘ—NXY or —(CH₂)ₙ—W
W is heterocycloalkyl containing at least one nitrogen atom;
Q is —C(O)—NH—;
X and Y are independently selected from the group consisting of hydrogen, (C₁-C₆)alkyl and heteroaryl-alkyl, or X and Y form together with the nitrogen atom on which they are attached, a heterocycloalkyl optionally substituted by (C₁-C₆)alkyl;
p is 0 or 1, and n is 0 or 1 and m is an integer from 0 to 6.

7. A compound of claim 6, wherein
the aryl, independently of Y′ and Z′ of R₁ is phenyl or naphthyl;
the heterocycloalkyl of W, is piperidine;
the (C₃-C₇)cycloalkyl independently of Y′ and Z′, is cyclohexyl;
the aryl, independently of Y′ and Z′ of R₂ is selected from the group consisting of phenyl, naphthyl and fluorenyl;
the heteroaryl of heteroaryl-alkyl, independently of X and Y, is pyridine;
the heterocycloalkyl formed together by X and Y with the nitrogen atom on which they are attached, is piperazine or pyrrolidine;
the heteroaryl, independently of Y′ and Z′ of R₂ is selected from the group consisting of thienyl, furyl, benzothienyl, pyridine, indolyl and thiadiazolyl; and
the aryl by of Y″ is phenyl.

8. A compound of claim 1 wherein
R₁ is —(CH₂)ₙ—[Q]ₚ—(CH₂)ₘ—NXY
Q is cyclohexyl;
X and Y are independently selected from the group consisting of hydrogen, and (C₁-C₆)alkyl, or X and Y form together with the nitrogen atom on which they are attached, piperidine;
n is 0 or 1, p is 0 or 1 and m is an integer from 1 to 6;
R₂ —(CH₂)_{n′}[Q′]_{p′}[C(X′)(Y′)]_{m′}Z′;
Q′ is —O—;
X′ is hydrogen;
Y′ is hydrogen or phenyl;
Z′ is selected from the group consisting of hydrogen, (C₁-C₆)alkyl, amino, cyclohexyl, phenyl, naphthyl, fluorenyl, thienyl, furyl, benzothienyl, thiadiazole, indolyl, quinolyl, quinoxalyl, isoquinolyl, pyrazinyl, xanthenyl or naphthyridyl; the phenyl, naphthyl, quinolyl and thiadiazolyl being optionally substituted by at least one substituent selected from the group consisting of —(CH₂)_{q′}—X″—Y″, hydroxy, halo, nitro, (C₁-C₆)alkylamino, and di((C₁-C₆)alkyl)amino;
X″ is selected from the group consisting of —O—, —S— and a covalent bond;
Y″ is (C₁-C₆)alkyl optionally substituted by at least one identical or different halo, or phenyl optionally substituted by halo;
p′ is 0 or 1; n′ is an integer from 0 to 4; and m′ is an integer from 0 to 4;
R₃ is —(CH₂)[_{n′}[Q′]_{p′}][C(X′)(Y′)]_{m′}Z′;
Q′ is —C(O)—;
X′ is hydrogen;
Y′ is selected from the group consisting of hydrogen, (C₁-C₆)alkyl, (C₁-C₆)alkoxy-carbonyl and phenyl;
Z′ is selected from the group consisting of hydrogen, (C₁-C₆)alkyl, (C₁-C₆)alkoxy-carbonyl, phenyl, naphthyl, fluorenyl, indolyl, benzothienyl, 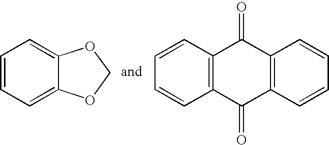
the phenyl, benzothienyl and indolyl being optionally substituted by at least one substituent selected from the group consisting of —(CH₂)_{q}′—X″—Y″, (C₁-C₆)alkoxy, halo, nitro, cyano and di((C₁-C₆)alkyl)amino;
X″ is selected from the group consisting of —O—, —C(O)—, —C(O)—O—, —SO₂— and a covalent bond;
Y″ is (C₁-C₆)alkyl optionally substituted by at least one identical or different halo, or phenyl;
p′ is 0 or 1; n′ and m′ are an integer from 0 to 6.

9. A compound claim 1 wherein
R₁ is —(CH₂)_{n′}[Q′]_{p′}[C(X′)(Y′)]_{m′}Z′;
X′ is hydrogen;
Y′ is hydrogen or phenyl;
Z′ is selected from the group consisting of hydrogen, (C₁-C₆)alkyl, phenyl optionally substituted by at least one identical or different halo and naphthyl;
p′ is 0, n′ is 0 or 1, and m′ is an integer from 0 to 6;
R₂ is pyrrolydinyl or —(CH₂)ₙ—[Q]ₚ—(CH₂)ₘ—NXY wherein
Q is —C(Z_{q})(Z_{q}′)—;
Z_{q} is hydrogen and Z_{q}′ is selected from the group consisting of hydrogen, phenyl optionally substituted by phenyl, cyclohexyl-methyl and benzyl;
X and Y is hydrogen;
p is 0 or 1, and n is 0 or 1, and m is an integer from 0 to 6;
R₃ is —(CH₂)_{n′}[Q′]_{p′}[C(X′)(Y′)]_{m′}Z′;
X′ is hydrogen;
Y′ is selected from the group consisting of hydrogen, (C₁-C₆)alkyl and (C₁-C₆)alkyl and (C₁-C₆)alkoxy-carbonyl;
Z′ is selected from the group consisting of hydrogen, (C₁-C₆)alkyl, (C₁-C₆)alkoxy-carbonyl, phenyl, naphthyl, fluorenyl, and
the phenyl being optionally substituted by at least one substituent selected from the group consisting of —(CH₂)_{q}′X″Y″, halo, nitro; and cyano;
X″ is selected from the group consisting of —O—, —C(O)—, —C(O)—O— and a covalent bond;
Y″ is alkyl optionally substituted by at least one identical or different halo, or a phenyl; and
p′ is 0, n′ and m′ is an integer from 0 to 6.

10. R₁ is —(CH₂)_{n′}[Q′]_{p′}[C(X′)(Y′)]_{m′}Z′;
X′ is hydrogen;
Y′ is hydrogen or phenyl;
Z′ is selected from the group consisting of hydrogen, (C₁-C₆)alkyl, naphthyl, and phenyl optionally substituted by at least one substituent selected from the group consisting of halo, amino and phenyl;
p′ 0, n′ is 0 or 1, and m′ is an integer from 0 to 6;
R₂ is —(CH₂)_{n′}[Q′[_{p′}[C(X′)(Y′)]_{m′}Z′;
X′ and Y′ are hydrogen;
Z′ is selected from the group consisting of hydrogen, (C₁-C₆)alkyl, phenyl, naphthyl, pyridine and benzothienyl,
the phenyl being optionally substituted by at least one —(CH₂)_{q′}—X″—Y″;
X″ is —O— or a covalent bond;
Y″ is (C₁-C₆)alkyl optionally substituted by at least one identical or different halo, or phenyl;
p′ represents is 0, n′ represents is 0 or 1, and m′ represents is an integer from 0 to 6;
R₃ is piperidine or —(CH₂)ₙ—[Q]ₚ—(CH₂)ₘ—NXY in which
Q is —C(O)—NH—;
X is hydrogen or (C₁-C₆)alkyl;
Y is hydrogen, (C₁-C₆)alkyl, and (pyridine)-ethyl, or X and Y form together with the nitrogen atom on which they are attached, piperazine optionally substituted by (C₁-C₆)alkyl;
p is 0 or 1, and n is 0 or 1 and m is an integer from 0 to 6.

11. A compound of claim 1, wherein one of R₁ or R₃ is —(CH₂)ₙ—[Q]ₚ—(CH₂)ₘ—NXY or —(CH₂)ₙ—W,
W is heterocycloalkyl containing at least one nitrogen atom;
Q is selected from the group consisting of —O—, —S—, —C(O)—NH—, —C(Z_{q})(Z_{q}′)—, aryl and (C₃-C₇)cycloalkyl;
Z_{q} and Z_{q}′ represent, are independently, selected from the group consisting of hydrogen aryl optionally substituted by aryl, (C₃-C₇)cycloalkyl-alkyl, arylalkyl, —C(O)O—R and —C(O)—NH—R′;
R is selected from the group consisting of (C₁-C₆)alkyl, aryl and aralkyl, aryl and aryl-alkyl being optionally substituted by at least one substituent selected from the group consisting of (C₁-C₆)alkoxy, hydroxy, halo, nitro., cyano, amino, (C₁-C₆)alkylamino and di((C₁-C₆)alkyl)amino;
R′ is selected from the group consisting of (C₁-C₆)alkyl, aryl, aralkyl, heteroaryl or heteroaryl-alkyl, the aryl, aralkyl, heteroaryl and heteroaryl-alkyl being optionally substituted by at least one substituent selected from the group consisting of (C₁-C₆)alkoxy, hydroxy, halo, nitro cyano, amino, (C₁-C₆)alkylamino, and di((C₁-C₆)alkyl)amino;
X and Y, are independently, selected from the group consisting of hydrogen, (C₁-C₆)alkyl, (C₁-C₆)alkoxy-carbonyl and heteroaryl-alkyl, or X and Y form together with the nitrogen atom on which they are attached, a heterocycloalkyl optionally substituted by (C₁-C₆)alkyl;
p is 0 or 1; n and m are independently an integer from 0 to 6.

12. A compound of claim 11, wherein
R₁ is —(CH₂),-[Q]ₚ—(CH₂)ₘ—NXY,
Q is aryl or (C₃-C₇)cycloalkyl;
X and Y are independently selected from the group consisting of hydrogen and (C₁-C₆)alkyl, or X and Y form together with the nitrogen atom on which they are attached, a heterocycloalkyl optionally substituted by (C₁-C₆)alkyl;
p represents is 0 or 1, and n and m are, independently, an integer from 0 to 6.

13. claim 11,
R₃ is —(CH₂)ₙ—[Q]ₚ—(CH₂)ₘ—NXY or —(CH₂)ₙ—W,
W is heterocycloalkyl containing at least one nitrogen atom;
Q is —C(O)—NH—;
X and Y are independently selected from the group consisting of hydrogen, (C₁-C₆)alkyl and heteroaryl-alkyl, or X and Y form together with the nitrogen atom on which they are attached, heterocycloalkyl optionally substituted by (C₁-C₆)alkyl;
p represents is 0 or 1, and n is 0 or 1 and m is an integer from 0 to 6.

14. A compound of claim 11 wherein R₂ is —(CH₂)ₙ-[Q′]_{p′}[C(X′)(Y′)]_{m′}Z′, in which
Q′ is —O—;
X′ is hydrogen;
Y′ and Z′ are independently selected from the group consisting of hydrogen, (C₁-C₆)alkyl, cyano, amino, (C₃-C₇)cycloalkyl, aryl and heteroaryl;
the aryl and heteroaryl being optionally substituted by at least one substituent selected from the group consisting of —(CH₂)_{q′}—X″—Y″, hydroxy, halo, nitro, amino, (C₁-C₆)alkylamino, and di((C₁-C₆)alkyl)amino;
X″ is selected from the group consisting of —O—, —S— and a covalent bond;
Y″ is (C₁-C₆)alkyl optionally substituted by at least one identical or different halo, or aryl optionally substituted by at least one identical or different halo radicals;
p′ is 0 or 1; n′ is 0, 1 or 2; and m′ is an integer from 0 to 6.

15. A compound of claim 11 wherein R₁ is —(CH₂)ₙ—[Q]ₚ—(CH₂)ₘ—NXY, wherein
X and Y are, independently, hydrogen or (C₁-C₆)alkyl;
p and n are 0, and m is an integer from 2 to 6.

16. A compound of claim 11 wherein R₂ is selected from the group consisting of naphthyl, phenyl, benzothienyl, quinoxalyl, quinolyl, isoquinolyl or indolyl, the phenyl, naphthyl and quinolyl being optionally substituted by at least one member selected from the group consisting of (C₁-C₆)alkoxy, halo, nitro, hydroxy, and (C₁-C₆)alkyl, the (C₁-C₆)alkyl itself being optionally substituted by at least one identical or different halo.

17. A compound of claim 11 wherein R₃ is —(CH₂)_{n′}[Q′]_{p′}[C(X′)(Y′)]_{m′}Z′,
X′ and Y′ are hydrogen;
Z′ represents is indolyl or benzothienyl; the indolyl being optionally substituted by at least one substituent selected from the group consisting of —(CH₂)_{q}—X″—Y″, (C₁-C₆)alkoxy or halo;
X″ is —SO₂— or a covalent bond;
Y″ is phenyl or alkyl optionally substituted by at least one identical or different halo;
q′ is 0 or 1; p′ is 0; n′ is 0 or 1; and m′ is 0 or 1.

18. A compound of claim 1, wherein R₁ is —(CH₂)ₙ —[Q]ₚ—(CH₂)ₘ—NXY
Q is cyclohexyl;
X and Y are, independently, hydrogen or (C₁-C₆)alkyl, or X and Y form, together with the nitrogen atom on which they are attached, piperadine;
n is 0 or 1, p is 0 or 1 and m is an integer from 1 to 6.

19. A compound of claim 1 wherein R₂ is —(CH₂)_{n′}[Q′]_{p′}[C(X′)(Y′)_{m′}Z′;
Q′ is —O—;
X′ is hydrogen;
Y′ is hydrogen or phenyl;
Z′ is selected from the group consisting of hydrogen, (C₁-C₆)alkyl, amino, cyclohexyl, phenyl, naphthyl, fluorenyl, thienyl, furyl, benzothienyl, thiadiazole, indolyl, quinolyl, quinoxalyl, isoquinolyl, pyrazinyl, xanthenyl and naphthhyridyl; the phenyl, naphthyl, quinolyl and thiadiazolyl being optionally substituted by at least one substituent selected from the group consisting of —(CH₂)_{q}—X″—Y′, hydroxy, halo, nitro, (C₁-C₆)alkylamino, and di((C₁-C₆)alkyl)amino;
X″ is selected from the group consisting of —O—, —S— and a covalent bond;
Y″ is (C₁-C₆)alkyl optionally substituted by at least one identical or different halo, or phenyl optionally substituted by halo;
p′ is 0 or 1; n′ is 0, 1 or 2; and m′ is an integer from 0 to 4;

20. A compound of claim 1 wherein R₃ is —(CH₂)_{n′}[Q′]_{p′}[C(X′)(Y′)_{m′}Z′;
Q′ is —C(O)—;
X′ is hydrogen;
Y′ the is selected from the group consisting of hydrogen, (C₁-C₆)alkyl, (C₁-C₆)alkoxy-carbonyl and phenyl;
Z′ is selected from the group consisting of hydrogen, (C₁-C₆)alkyl, (C₁-C₆)alkoxy-carbonyl, phenyl, naphthyl, fluorenyl, indolyl, benzothienyl, 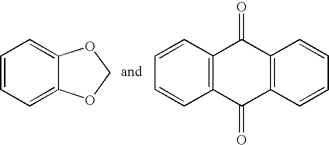
the phenyl, benzothienyl and indolyl being optionally substituted by at least one substituent selected from the group consisting of —(CH₂)_{q}—X″—Y″, (C₁-C₆)alkoxy, halo, nitro, cyano, and di((C₁-C₆)alkyl)amino;
X″ is selected from the group consisting of —O—, —C(O)—, —C(O)—O—, —SO₂— and a covalent bond;
Y″ is (C₁-C₆)alkyl optionally substituted by at least one identical or different halo, or phenyl;
p′ is 0 or 1; n′ is 0, 1 or 2; and m′ is an integer from O to 6.

21. A compound of claim 1 wherein
R₁ is —(CH₂)ₙ—[Q]ₚ—(CH₂)ₘ—NXY,
X and Y are, independently, hydrogen or (C₁-C₆)alkyl;
p and n are 0, m is an integer from 2 to 6;
R₂ is selected from the group consisting of quinoxalyl, quinolyl and naphthyl, the quinolyl and naphthyl being optionally substituted by at least one member selected from the group consisting of (C₁-C₆)alkyl, (C₁-C₆)alkoxy and halo;
R₃ is —(CH₂)_{n′}[Q′]_{p′}[C(X′)(Y′)]_{m′}Z′ in which
X′ and Y′ are hydrogen;
Z′ is indolyl optionally substituted by at least one substituent selected from the group consisting of —(CH₂)_{q′}—X″—Y″, (C₁-C₆)alkoxy of and halo;
X″ is a covalent bond;
Y″ is alkyl optionally substituted by at least one identical or different halo;
q′ is 0 or 1; p′ is 0; n′ is 0 or 1; and m′ is 0 or 1.

22. A process for the preparation, in liquid phase, of a compound of claim 1, comprising reacting
an isothiocyanate of the formula R₁—NCS with a hydrazide of the formula R₂—C(O)—NH—NH₂ in which R₁ and R₂ have the meaning of claim 2, to obtain a compound of the formula 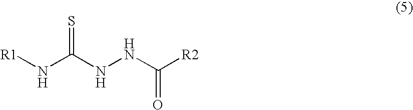
subjecting a compound of formula (5) to a basic treatment to obtain a compound of the formula 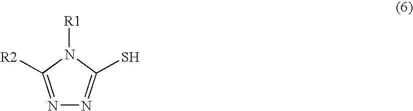
and reacting a compound of formula (6) with
A) either a compound of the formula Br—(CH₂)_{n′}[Q′]_{p′}[C(X′)(Y′)_{m′}Z′ where n′=1, p′=m′=0 and Z′ has the meaning of claim 1 to obtain, after deprotection of the amine function present on the molecule, the corresponding compound of formula (I), or with
B) a compound of the formula Br—(CH₂)_{n′}[Q′]_{p′}[C(X′)(Y′)_{m′}Z′ wherein n′=1, Q′=—C(O)—, m′=0 and Z′ has the meaning of claim 1 to obtain, after deprotection of the amine function present on the molecule, the corresponding compound of formula (I), or with
C) a compound of the formula Br—(CH₂)_{n′}[Q′]_{p′}[C(X′)(Y′)]ₘ, Z′ where Q′, X′, Y′, Z′, n′, p′ and m′ have the meaning of claim 1 to obtain, after deprotection of the amine function present on molecule, the corresponding compound of formula (I).

23. A pharmaceutical composition containing, as active ingredient, a compound of claim 1, in combination with a pharmaceutically acceptable carrier.

24. A pharmaceutical composition containing, as active ingredient, in combination with a pharmaceutically acceptable support, at least one compound of the formula 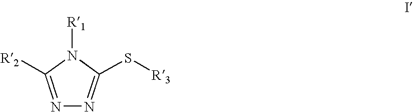
in racemic, enantiomeric form or all combinations of these forms, in which
one of R′₁, R′₂ or R′₃ is —(CH₂)ₙ—[Q]ₚ—(CH₂)ₘ—NXY or —(CH₂)ₙ—W,
W is heterocycloalkyl containing at least one nitrogen atom;
Q is selected from the group consisting of —O—, —S—, —C(O)—NH—, —C(Z_{q})(Z_{q}′)—, aryl and (C₃-C₇)cycloalkyl;
Z_{q} and Z_{q′} are independently, selected from the group consisting of the hydrogen, aryl optionally substituted by aryl, (C₃-C₇)cycloalkyl-alkyl, arylalkyl, —C(O)O—R and —C(O)—NH—R′;
R is (C₁-C₆)alkyl, aryl or aralkyl, aryl and aralkyl being optionally substituted by at least one
substituent selected from the group consisting of (C₁-C₆)alkoxy, hydroxy, halo, nitro, cyano, amino, (C₁-C₆)alkylamino and di((C₁-C₆)alkyl)amino;
R′ is selected from the group consisting of (C₁-C₆)alkyl, aryl, aralkyl, heteroaryl and heteroaryl-alkyl, the aryl, aralkyl, heteroaryl and heteroaryl-alkyl being optionally substituted by at least one substituent selected from the group consisting of (C₁-C₆)alkoxy, hydroxy, halo, nitro, cyano, amino, (C₁-C₆)alkylamino and di((C₁-C₆)alkyl)amino;
X and Y are independently selected from the group consisting of hydrogen, (C₁-C₆)alkyl, (C₁-C₆)alkoxy-carbonyl and heteroaryl-alkyl, or X and Y form together, with the nitrogen atom on which they are attached, a heterocycloalkyl optionally substituted by (C₁-C₆)alkyl;
p is 0 or 1; n and m are, independently, an integer from 0 to 6;
and the two other Rs are, independently, —(CH₂)_{n′}[Q′]_{p′}[C(X′)(Y′)_{m′}Z′,
Q′ is selected from the group consisting of —O—, —S—, —C(O)—, —NH—, —CH═CH— and —C≡C—;
X′, Y′ and Z′ are independently selected from the group consisting of hydrogen, (C₁-C₆)alkyl, (C₁-C₆)alkoxy, (C₁-C₆)alkoxy-carbonyl, cyano, amino, (C₁-C₆)alkylamino, di((C₁-C₆)alkyl)amino, (C₃-C₇)cycloalkyl, heterocycloalkyl, aryl, heteroaryl, 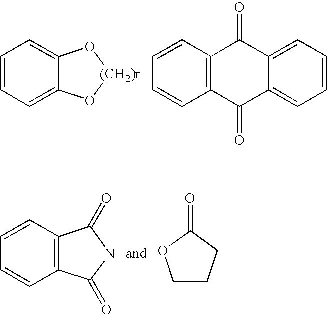
r is 1, 2 or 3
the (C₃-C₇)cycloalkyl, heterocycloalkyl, aryl and heteroaryl being optionally substituted by at least one substituent selected from the group consisting of —(CH₂)_{q′}—X″—Y″, hydroxy, halo, nitro, cyano, amino, (C₁-C₆)alkylamino and di((C₁-C₆)alkyl)amino;
X″ is selected from the group consisting of —O—, —S—, —C(O)—, —C(O)—O—, —SO₂— and a covalent bond;
Y″ is (C₁-C₆)alkyl optionally substituted by at least one identical or different halo; or aryl or heteroaryl optionally substituted by at least one substituent selected from the group consisting of (C₁-C₆)alkoxy, hydroxy, halo, nitro, cyano, amino, (C₁-C₆)alkylamino and di((C₁-C₆)alkyl)amino;
p′ is 0 or 1, and n′, m′ and q′ are, independently, an integer from 0 to 6.
